(19) United States
(12) Patent Application Publication
(10) Pub. No.: US 2009/0031455 A1
(43) Pub. Date: Jan. 29, 2009

(54) TERPENE HYDROXYLATION

(75) Inventors: Asaph Aharoni, Tel Aviv (IL); Maarten Anthonie Jongsma, Wageningen (NL); Hendrik Jan Bouwmeester, Renkum (NL)

Correspondence Address: FOLEY AND LARDNER LLP;SUITE 500, 3000 K STREET NW, WASHINGTON, DC, 20007, US

(73) Assignee: PLANT RESEARCH INTERNATIONAL B.V., Wageningen (NL)

(21) Appl. No.: 11/577,082

(22) PCT Filed: Oct. 14, 2005
(86) PCT No.: PCT/NL05/50014

§ 371 Date: Jul. 16, 2007

Related U.S. Application Data
(60) Provisional application No. 60/618,821, filed on Oct. 14, 2004.

(30) Foreign Application Priority Data
Oct. 14, 2004 (EP) 04077854.0

(51) Int. Cl. A01H 5/00; C12N 1/21; C12N 7/01; C12N 1/15; C12P 7/04; C12N 5/10; C12N 5/04

(52) U.S. Cl. 800/301; 435/252.3; 435/235.1; 435/254.11; 435/348; 435/325; 435/410; 800/298; 435/157

(57) ABSTRACT

The present invention provides a novel cytochrome P450 enzyme, as well as vectors and recombinant host cells comprising the gene encoding the enzyme. Provided are recombinant plant with enhanced disease and/or pest resistance, modified favour and/or fragrance and methods for hydroxy lifting terpene substrates.

### FIELD OF THE INVENTION

The present invention relates to a novel cytochrome P450 monooxygenase enzyme, the nucleic acid and amino acid sequences thereof, functional variants and fragments of those sequences, as well as nucleic acid vectors and recombinant host cells and organisms producing at least one functional enzyme according to the invention. Further provided are in vitro and in vivo methods for hydroxylating terpene substrates, especially monoterpene substrates and/or aromatic hydrocarbons, using one or more enzymes according to the invention. Such methods include the use of the enzyme(s) in the production of perillyl alcohol for cancer prophylaxis and therapy, the in vitro or in vivo production of hydroxylated terpenes such as for example myrtenol and/or myrtenol derivatives, gene therapy methods and transgenic plants, plant tissues, cells and organs with modified flavor/fragrance properties and/or enhanced pest/pathogen resistance and/or anti-carcinogenic properties. Nutritional- (food or food supplements, including functional foods or nutraceuticals), cosmetic- (e.g. creams, perfumes, etc.), pest attracting- (e.g. beetle attractants) or repelling-, antipathogenic- or pharmaceutical-compositions comprising suitable amounts of one or more hydroxylated terpenes or terpene analogues (or derivatives), obtainable by contacting a suitable terpene substrate with a functional enzyme according to the invention, are also provided. The present invention, thus, covers various uses of the nucleic acids and proteins according to the invention, which range from the field of biotechnology to medical applications.

### BACKGROUND OF THE INVENTION

The majority of enzymes used in bioconversion employing biocatalysis for industrial and laboratory applications is obtained from microbial sources, while a minor fraction of enzymes is obtained from plant sources (Faber, 2000, Biotransformation in Organic Chemistry, 4^{th} ed. Springer-Verlag, Berlin). Nevertheless, the plant kingdom is an important source for the chemist and the biotechnologist because plants produce a unique variety of chemicals (Franssen and Walton, 1999, in Chemicals for Plants, editors Walton and Brown, World Scientific Publishers, London, p 277-325).

By far the most important group of plant secondary metabolites, containing a vast number of components that act as flavour, fragrance, pharmaceutical or bioactive (insecticidal, anti-microbial, repellent, attractant, etc.) compounds, are the terpenoids. The terpenoids belong to the isoprenoids. By definition isoprenoids are made up of so-called isoprene (C5) units. This can be recognized in the number of C-atoms present in the isoprenoids which usually can be divided by five (C5, C10, C15, C20, C25, C30 and C40), although also irregular isoprenoids (e.g. C13, C16) and polyterpenes (Cn) have been reported.

The terpenoids consist, amongst others, of monoterpenes (C10), sesquiterpenes (C15), diterpenes, triterpenes, tetraterpenes and polyterpenes (rubbers), etc. Mono- and sesquiterpenes, the C10 and C15 branch of the isoprenoid family, are economically interesting as flavor and fragrance compounds in foods and cosmetics, and can have anti-carcinogenic effects and antimicrobial properties. Terpenoids, and mainly C10 and C15 members of this family, have been identified at varying levels in the flavour profiles of most if not all soft fruit (Maarse 1991, Volatile compounds in food and beverages, New York, Marcel Dekker Inc.). For example, most citrus species are rich in various terpenoid components (Weiss 1997, Essential Oil Crops, Wallingford, Cab International). Another example is mango, in which terpenes comprise the main volatiles of most cultivars studied to date (Macleod and Pieris 1984, Phytochemistry 23, 361-366). Mono- and sesquiterpenes have also been shown to be of ecological significance, for instance in the interaction and signalling between plants, plants and insects/spider mites and plants and microorganisms.

All plant synthesized terpenoids are derived from either the mevalonate pathway active in the cytosol or the plastidial 2-C-methyl-D-erythritol 4-phosphate (MEP) pathway (Rodriguez-Concepcion and Boronat, 2002) (see FIG. 1). Both pathways lead to the formation of isopentenyl diphosphate (IPP) and its allylic isomer dimethylallyl diphosphate (DMAPP), the basic terpenoid biosynthesis building blocks. In both compartments, IPP and DMAPP are utilized by 4 prenyltransferases in condensation reactions producing prenyl diphosphates. Condensation of IPP and DMAPP catalyzed by the prenyltransferase geranyl diphosphate (GPP) synthase yields GPP, the immediate precursor of monoterpenes. The condensation of two IPP units with one DMAPP by the action of farnesyl diphosphate (FPP) synthase generates the precursor for sesquiterpene biosynthesis. Following the formation of these precursors, the various monoterpenes and sesquiterpenes are generated through the action of terpenoid synthases (TPS; Trapp and Croteau, 2001, Genetics 158, 811-832). Primary terpene skeletons formed by TPS might be further modified by hydroxylation, oxidation, double bond reduction, acylation, glycosylation and methylation (Lange and Croteau, 1999, Plant Biol. 2, 139-144). The complexity of terpenoid biosynthesis is further increased by subcellular compartmentation of the enzymes involved (Cunillera et al., 1997, J. Biol. Chem. 272, 15381-15388). For example, multiple isoforms of the gene for FPP synthase have been detected in Arabidopsis, with FPS1S and FPS2 encoding cytosol-targeted proteins while FPS1L encodes a mitochondrially targeted protein (Cunillera et al., 1997, supra). The FPS1 gene is bifunctional and uses alternative transcription start sites or selection of alternative translation initiation codons to generate either the cytosolic isoform (FPS1S) or the mitochondrial isoform (FPS1L).

While studying flavour biosynthesis in wild and cultivated strawberry, the present inventors cloned a novel enzyme, able to catalyze the hydroxylation of the monoterpene α-pinene at the C10 position, resulting in the formation of the monoterpene alcohol myrtenol. Myrtenol has been described as one of the few compounds which may contribute to the typical aroma of wild strawberries (Honkanen and Hirvi, 1990, The flavour of berries, Dev. Food. Sci. Amsterdam, Elsevier Scientific Public. 125-193). Myrtenol thus plays a role in flavour and fragrance biosynthesis, but has also been reported to have antimicrobial activity (Saito et al., 1996. Mokuzai-Gakkaishi, Journal of the Japan Wood Research Society 42: 677-680), to act as an attractant to certain insects, such as the pine shoot beetle, Tomicus piniperda (see U.S. Pat. No. 6,203,786) and as a repellent of other insect such as the old house borer, Hylotrupes bajulus (Fettkother et al., 2000. Chemoecology 10:1-10). Also a product of myrtenol, (1R,5S)-myrtenal, is known to be a deterrent for Aphis fabae, interfering with the normal attraction to the aphid's host plant (Hardie et al., 1994. J Chem Ecol 20: 2847-2855).

The novel enzyme is herein referred to as PINH (pinene hydroxylase or myrtenol synthase) and was found to be a member of the very large and diverse superfamily of cytochrome P450 enzymes. The DNA encoding the enzyme which carries out the preceding step, α-pinene synthase (PINS), has already been cloned from strawberry, see WO02/064764. Also, DNA encoding alcohol acyl transferases (AAT), which converts myrtenol to myrtenyl acetate has been cloned from various plant species, see WO00/32789, Aharoni et al. 2000 (The Plant Cell Vol. 12, 647-661) and Beekwilder et al. 2004 (Plant Physiol. 135: 1865-1878).

Cytochrome P450 enzymes are widely distributed and are found in all organisms, ranging from bacteria to humans (Nelson, 1999, Arch Biochem Biophys 369, 1-10). Arabidopsis alone contains 272 cytochrome P450 genes, the function of most of which remains unknown (Werck-Reichhart et al. The Arabidopsis Book, 2002; Schuler and Werck-Reichhart, Annu. Rev. Plant. Biol. 2003, 54:629-667). Cytochrome P450 enzymes (Mr=50,000) mostly catalyze oxidation reactions, but also reductions. Cytochromes P450s strongly absorb light at 450 nm when they are in the reduced state and complexed with CO (carbon monoxide). Light of 450 nm displaces CO from the heme, hence CO binding is photoreversible. For this reason, cytochrome P450 enzymes exhibit photoreversible inhibition by CO (Donaldson and Luster, 1991, Plant Physiol 96: 669-674).

Plant cytochrome P450 monooxygenase systems are associated with the endoplasmic reticulum (ER) or a prevacuole, and consequently are located in the membrane (microsomal) fraction of the cell, which can be isolated by centrifugation of homogenized cells using known methods (in contrast, bacterial cytochrome P450s are soluble proteins). Most classical plant P450 enzymes have an N-terminal trans-membrane helix domain, anchoring the protein to the ER, with the remaining part of the protein being in the cytosol. For catalytic activity cytochrome P450 enzymes need to be coupled to an electron donating protein, such as a cytochrome P450 reductase or a cytochrome b5, which enables electron transfer from NADPH into the catalytic site of the enzyme. The electron donating protein is also anchored to the ER via its N- or C-terminus. For reviews see Werck-Reichhart et al. 2002 and Schuler and Werck-Reichhart 2003, supra).

A large number of plant cytochrome P450 genes, encoding enzymes with diverse function and with diverse substrate specificity, have been cloned to date. As already mentioned, the in vivo function of many of these enzymes remains unknown. A complicating factor in function determination is that there seems to be little correlation between the primary structure (the amino acid sequence) and the function, while secondary and tertiary structures are conserved, but difficult to analyse. Tertiary structure analysis has revealed that helices and random loop regions surrounding the catalytic core of the enzyme contribute to the substrate specificity, while the amino acid residues that constitute the active site of the enzyme vary widely between different cytochromes P450; however, the principal component of the active site of all these enzymes is a heme moiety. The iron ion of the heme moiety is the site of the catalytic reaction, and is also responsible for the strong 450 nm absorption peak in combination with CO. The substrate specificity of cytochrome P450 enzymes depends on their function in the organism: the biosynthesis of metabolites or the breakdown of xenobiotics. Enzymes that are involved in biosynthetic routes of metabolites in plants or animals in general have a very high substrate specificity (Donaldson and Luster, 1991, supra; Mihaliak et al., 1993, Cytochrome P-450 terpene hydroxylases; In P. J. Lea ed, Methods in Plant Biochemistry, Enzymes of Secondary Metabolism, Vol 9. Academic Press, Londen, pp 261-279; Schuler, 1996, Crit. Rev Plant Sci 15:235-284). It was, therefore, rather surprising that the plant PINH enzyme according to the present invention could also use other terpenes or terpene analogues as substrate. In particular, PINH was found to hydroxylate monoterpenes at the allylic methyl group (C10 or C7 in α-pinene- and limonene-like monoterpene structures, respectively and in the structurally equivalent position of other terpenes and aromatic hydrocarbons). For example PINH was able to specifically hydroxylate (+) and/or (−) limonene at the C7 position leading to the production of (+) and/or (−) perillyl alcohol. In addition to α-pinene and limonene, PINH 7-hydroxylated α-phellandrene, α-terpinolene, α-terpinene and p-cymene. Since p-cymene is aromatic this suggested that also other aromatic substrates are C7-hydroxylated by PINH, for example toluene to yield benzyl alcohol, a colorless liquid with weak, slightly sweet odour and constituent of many essential oils both free or as ester that is used in perfumery and flavour industries and as an anti-microbial preservative in pharmaceuticals and cosmetics.

In addition, despite the reported high regio-selectivity of cytochrome P450 enzymes also some hydroxylation occurred in different regions. For example, limonene was also converted to limonen-10-ol and α-terpinolene to tentatively identified 7-OH and 10-OH alcohols.

The enzymes provided herein have thus unique properties, which can be suitably used in various fields, such as the production/modification of flavour and/or fragrances, of pharmaceutically active compounds (such as perillyl alcohol), in the production of bio-control agents and for engineering plant resistance.

### GENERAL DEFINITIONS

“Myrtenol” has the chemical formula as depicted in FIGS. 2 and 4. Synonyms used are for example 10-hydroxy-2-pinene and 2-pinen-10-ol. The odor has been described as campherous, minty, medicinal, woody.

“Myrtenyl acetate” has the chemical formula as depicted in FIG. 2. Synonyms used are for example 2,2-pinene-10-yl acetate and 2-pinen-10-ol acetate. The odor of myrtenyl acetate has been described as fresh, woody, minty and the taste as fresh, woody, herbaceous, carrot.

“Flavour” refers herein to the taste, while “fragrance” refers to the odor.

“Enantiomers” refers to a pair of molecular entities which are mirror images of each other and non-superposable. They are designated by a (+) or (−) prefix. Enzymes may have a higher substrate specificity for one particular enantiomer, for example (−) alpha-pinene. A “racemate” is an equimolar mixture of a pair of enantiomers and is designated by the prefix (±).

“Terpenes” are hydrocarbons having a carbon skeleton derived from isoprene and are subdivided into groups based on their carbon number, e.g. C10 monoterpenes, C15 sesquiterpenes, C20 diterpenes, C25 sesterterpenes, C30 triterpenes, C40 tetraterpenes and C5n polyterpenes. They are herein generally referred to by their trivial names, as e.g. described in Encyclopedia of Chemical Technology, Fourth Edition, Volume 23, page 834-835.

“Terpenoids” refer to oxygen containing terpene analogues. Terpenoids are subdivided in the same manner as terpenes. Thus, monoterpenoids are terpenoids having a C10 skeleton. Terpenes and terpenoids are used herein interchangeably. Terpene analogous include for example terpene derivatives such as alcohols, esters, aldehydes and ketones. Especially, in terpene analogue substrates additional functional groups may be present at positions other than the carbon position which is hydroxylated by the PINH enzyme. Monoterpenes may further be distinguished by the structure of the carbon skeleton and may be grouped into “acyclic monoterpenes” (e.g. myrcene, (Z)- and (E)-ocimene, linalool, geraniol, nerol, citronellol, myrcenol, geranial, citral a, neral, citral b, citronellal, etc.), “monocyclic monoterpenes” (e.g. limonene, α- and γ-terpinene, α- and β-phellandrene, terpinolene, menthol, carveol, etc.), “bicyclic monoterpenes” (e.g. α-pinene, β-pinene, myrtenol, myrtenal, verbanol, verbanon, pinocarveol, etc.) and “tricyclic monoterpenes” (e.g. tricyclene). See Encyclopedia of Chemical Technology, Fourth Edition, Volume 23, page 834-835.

The carbon skeleton numbering used herein for terpenes and terpene analogous is depicted in FIG. 4, exemplified by alpha pinene and limonene, wherein the head-to-tail orientation of the two isoprene units is depicted top (head) to bottom (tail). The “C10 position (head) analogous to alpha-pinene” refers to the structurally analogous position in any other (mono)terpene molecule. Similarly, the “C10 and/or C9 position (tail), or the C7 position (head) analogous to limonene” refers to the structurally analogous position in any other (mono)terpene. In addition the same numbering is used for aromatic hydrocarbons and derivatives comprising a single aromatic ring (benzyl ring or 6 carbon ring), such as toluene (see FIG. 5). Preferably, the enzymes according to the invention do not hydroxylate carbons of the 6 carbon ring.

“Catalysis” or “biocatalysis” refers to the catalytic activity, while “catalyst” or “biocatalyst” refers to the enzyme, cell/organism or composition comprising the catalytic activity.

“Bio-control” refers herein to the protection of organisms (e.g. plants or animals) against damage or disease caused by other organisms, such as insects, herbivores (e.g. geese, deer, rabbits), plant or animal pests or pathogens, damage causing microorganisms (e.g. bacteria, viruses, fungi), etc. Bio-control agents may cause such protection by external application (e.g. plant protection agents) or incorporation into products (e.g. into food or feed products) or by in vivo production of hydroxylated PINH products or one or more derivatives thereof or by gene silencing of endogenous PINH gene(s) (e.g. transgenic plants with enhanced pest and/or pathogen resistance levels).

The term “nucleic acid sequence” (or nucleic acid molecule) refers to a DNA or RNA molecule in single or double stranded form, particularly a DNA encoding a protein or protein fragment according to the invention. An “isolated nucleic acid sequence” refers to a nucleic acid sequence which is no longer in the natural environment from which it was isolated, e.g. the nucleic acid sequence in a bacterial host cell or in the plant nuclear or plastid genome.

The terms “protein” or “polypeptide” are used interchangeably and refer to molecules consisting of a chain of amino acids, without reference to a specific mode of action, size, 3-dimensional structure or origin. A “fragment” or “portion” of a PINH protein may thus still be referred to as a “protein”. An “isolated protein” is used to refer to a protein which is no longer in its natural environment, for example in vitro or in a recombinant bacterial or plant host cell. An enzyme is a protein comprising enzymatic activity.

The term “gene” means a DNA sequence comprising a region (transcribed region), which is transcribed into an RNA molecule (e.g. an mRNA) in a cell, operably linked to suitable regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′non-translated sequence comprising e.g. transcription termination sites.

A “chimeric gene” (or recombinant gene) refers to any gene, which is not normally found in nature in a species, in particular a gene in which one or more parts of the nucleic acid sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more coding sequences or to an antisense (reverse complement of the sense strand) or inverted repeat sequence (sense and antisense, whereby the RNA transcript forms double stranded RNA upon transcription).

“Expression of a gene” refers to the process wherein a DNA region, which is operably linked to appropriate regulatory regions, particularly a promoter, is transcribed into an RNA, which is biologically active, i.e. which is capable of being translated into a biologically active protein or peptide (or active peptide fragment) or which is active itself (e.g. in posttranscriptional gene silencing or RNAi). The coding sequence is preferably in sense-orientation and encodes a desired, biologically active protein or peptide, or an active peptide fragment. In gene silencing approaches, the DNA sequence is preferably present in the form of an antisense DNA or an inverted repeat DNA, comprising a short sequence of the target gene in antisense or in sense and antisense orientation. “Ectopic expression” refers to expression in a tissue in which the gene is normally not expressed.

A “transcription regulatory sequence” is herein defined as a nucleic acid sequence that is capable of regulating the rate of transcription of a (coding) sequence operably linked to the transcription regulatory sequence. A transcription regulatory sequence as herein defined will thus comprise all of the sequence elements necessary for initiation of transcription (promoter elements), for maintaining and for regulating transcription, including e.g. attenuators or enhancers. Although mostly the upstream (5′) transcription regulatory sequences of a coding sequence are referred to, regulatory sequences found downstream (3′) of a coding sequence are also encompassed by this definition.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is only active in specific types of tissues or cells.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous and, where necessary to join two protein encoding regions, contiguous and in reading frame so as to produce a “chimeric protein”. A “chimeric protein” or “hybrid protein” is a protein composed of various protein “domains” (or motifs) which is not found as such in nature but which a joined to form a functional protein, which displays the functionality of the joined domains (for example DNA binding or repression leading to a dominant negative function). A chimeric protein may also be a fusion protein of two or more proteins occurring in nature. The term “domain” as used herein means any part(s) or domain(s) of the protein with a specific structure or function that can be transferred to another protein for providing a new hybrid protein with at least the functional characteristic of the domain. For example, cytochrome P450 monooxygenases comprise an oxygen binding domain and a heme domain.

The terms “target peptide” refers to amino acid sequences which target a protein to intracellular organelles such as vacuoles, plastids, preferably chloroplasts, mitochondria, leucoplasts or chromoplasts, the endoplasmic reticulum, or to the extracellular space (secretion signal peptide). A nucleic acid sequence encoding a target peptide may be fused (in frame) to the nucleic acid sequence encoding the amino terminal end (N-terminal end) of the protein or may replace part of the amino terminal end of the protein.

A “nucleic acid construct” or “vector” is herein understood to mean a man-made nucleic acid molecule resulting from the use of recombinant DNA technology and which is used to deliver exogenous DNA into a host cell. The vector backbone may for example be a binary or superbinary vector (see e.g. U.S. Pat. No. 5,591,616, US2002138879 and WO9506722), a co-integrate vector or a T-DNA vector, as known in the art and as described elsewhere herein, into which a chimeric gene is integrated or, if a suitable transcription regulatory sequence is already present, only a desired nucleic acid sequence (e.g. a coding sequence, an antisense or an inverted repeat sequence) is integrated downstream of the transcription regulatory sequence. Vectors usually comprise further genetic elements to facilitate their use in molecular cloning, such as e.g. selectable markers, multiple cloning sites and the like (see below).

A “host cell” or a “recombinant host cell” or “transformed cell” are terms referring to a new individual cell (or organism) arising as a result of at least one nucleic acid molecule, especially comprising a chimeric gene encoding a desired protein or a nucleic acid sequence which upon transcription yields an antisense RNA or an inverted repeat RNA (or hairpin RNA) for silencing of a target gene/gene family, having been introduced into said cell. The host cell may be any eukaryotic or prokaryotic cell e.g. a plant cell, microbial, insect or mammal (including human) cell. The host cell may contain the nucleic acid construct as an extra-chromosomally (episomal) replicating molecule, or more preferably, comprises the chimeric gene integrated in the nuclear or plastid genome of the host cell. Included are any derivatives of the host cell, such as tissues, whole organism, cell cultures, explants, protoplasts, further generations, etc. derived from the cell which retain the introduced gene or nucleic acid.

The term “selectable marker” is a term familiar to one of ordinary skill in the art and is used herein to describe any genetic entity which, when expressed, can be used to select for a cell or cells containing the selectable marker. Selectable marker gene products confer for example antibiotic resistance, or herbicide resistance or another selectable trait such as a phenotypic trait (e.g. a change in pigmentation) or a nutritional requirements. The term “reporter” is mainly used to refer to visible markers, such as green fluorescent protein (GFP), eGFP, luciferase, GUS and the like.

The term “ortholog” of a gene or protein refers herein to the homologous gene or protein found in another species, which has the same function as the gene or protein, but is (usually) diverged in sequence from the time point on when the species harbouring the genes diverged (i.e. the genes evolved from a common ancestor by speciation). Orthologs of the PINH gene may thus be identified in other plant, animal, bacterial or fungal species based on both sequence comparisons (e.g. based on percentages sequence identity over the entire sequence or over specific domains) and/or functional analysis.

The terms “homologous” and “heterologous” refer to the relationship between a nucleic acid or amino acid sequence and its host cell or host organism, especially in the context of transgenic cells/organisms. A homologous sequence is thus naturally found in the host species (e.g. a tomato plant transformed with a tomato gene), while a heterologous sequence is not naturally found in the host cell (e.g. a tomato plant transformed with a sequence from potato plants). Depending on the context, the term “homolog” or “homologous” may alternatively refer to sequences which are descendent from a common ancestral sequence (e.g. they may be orthologs).

“Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tₘ) for the specific sequences at a defined ionic strength and pH. The Tₘ is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

“Sequence identity” and “sequence similarity” can be determined by alignment of two peptide or two nucleotide sequences using global or local alignment algorithms. Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length, maximizing the number of matches and minimises the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA. Alternatively percent similarity or identity may be determined by searching against databases such as FASTA, BLAST, etc.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”, e.g. “a cell” refers also to several cells in the form of cell cultures, tissues, whole organism, etc. It is further understood that, when referring to “sequences” herein, generally the actual physical molecules with a certain sequence of subunits (e.g. amino acids) are referred to.

### DETAILED DESCRIPTION OF THE INVENTION

When analyzing the headspace volatiles of cultivated (Fragaria x ananassa) and wild (Fragaria vesca) strawberry fruit using GC-MS, it was found that these emit different terpenes. Cultivated strawberry fruit produced the monoterpene linalool and the sesquiterpene nerolidol, whereas wild strawberry fruit emitted the monoterpenes α- and β-pinene, β-myrcene, α-terpineol, and β-phellandrene, as well as myrtenyl acetate, and low levels of myrtenol, which were not detected in the cultivated species (see FIG. 2).

It was found that the gene responsible for producing α-pinene in wild strawberry fruit (termed PINS) was not functional in cultivated strawberry. Following this discovery, the aim was to clone the gene responsible for the oxidation of α-pinene to myrtenol, as evidently occurred in wild strawberry. Initially five Fragaria x ananassa EST clones were identified which were putative targets for encoding the responsible enzyme. One of these clones, D59, encoded a cytochrome P450 of the CYP71 family.

Detailed gene expression analysis using the five different fragments as probes for RNA gel-blot hybridizations revealed that clone D59 showed increased expression in the ripe red strawberry fruit, but was also expressed, to even higher levels, in roots (data not shown). It had been reported that myrtenol glycoside is a component of cultivated strawberry roots (Wintoch, 1993, Flav. Frag. J. 6, 209-215) and this supported the view that the enzyme corresponding to the D59 gene was indeed a α-pinene hydroxylase.

The present inventors then analyzed the production of myrtenol in fruit and roots of various wild and cultivated strawberry species (see Table 1). The free form of myrtenol was detected in ripe fruit of four wild species, but not in any of the eight cultivated species examined. The same pattern was detected for glycosylated myrtenol and myrtenyl acetate. On the other hand, relatively high levels of free and glycosylated myrtenol (more than in ripe fruit tissue of the wild species) were detected in the roots of both species.

TABLE 1
Presence of free myrtenol, glycosidically bound myrtenol and myrtenyl acetate
in ripe fruit and roots of various wild and cultivated strawberries

| | | | | Glycosidically | Free form |
|---|---|---|---|---|---|
| | | | Free form | bound | (mg/kg) |
| | | | (mg/kg) | (mg/kg) | Myrtenyl |
| Species | Name | Tissue | Myrtenol | Myrtenol | acetate |
| wild | PRI line 92189 | ripe fruit | 0.109 | 0.04 | 0.155 |
| wild | PRI line H1 | ripe fruit | 0.608 | 0.09 | 0.392 |
| wild | PRI line 92190 | ripe fruit | 0.352 | 0.05 | 0.604 |
| wild | Yellow wonder | ripe fruit | 0.227 | n.d. | 0.418 |
| cultivated | Elsanta | ripe fruit | n.d. | n.d. | n.d. |
| cultivated | Calypso | ripe fruit | n.d. | n.d. | n.d. |
| cultivated | Camerosa | ripe fruit | n.d. | n.d. | n.d. |
| cultivated | Gorrela | ripe fruit | n.d. | n.d. | n.d. |
| cultivated | Sure crop | ripe fruit | n.d. | n.d. | n.d. |
| cultivated | Senga sengana | ripe fruit | n.d. | n.d. | n.d. |
| cultivated | Virginiana 352 | ripe fruit | n.d. | n.d. | n.d. |
| cultivated | Elsanta | roots | 7.230 | 0.239 | n.d. |
| wild | PRI line 92189 | roots | 4.634 | 0.271 | n.d. |

The protein encoded by the D59 gene showed the highest homology (49%-50% amino acid sequence identity) to three Arabidopsis proteins with unknown functions (CYP71A26, CYP71A25 and CYP71A22; Salamoubat et al. 2000, Nature 408: 820-822). As cytochrome P450 enzymes with at least 40% amino acid identity are classified within one family, the present enzyme is a novel CYP71 family member according to the commonly used nomenclature (Nelson et al. 1996, Pharmacogenetics 6, 1-42), while the subfamily classification remains to be determined. Enzyme assays with the recombinant D59 protein (termed FaPINH; SEQ ID NO: 4) produced in yeast microsomes showed that the substrate α-pinene was hydroxylated at C10 to form myrtenol (FIGS. 3-4). The (−)-α-pinene form was preferred to (+)-α-pinene as a substrate, which matched the finding that the monoterpene synthase PINS of wild strawberry mainly produced the (−)-α-pinene enantiomer (an enantiomeric excess of over 99% of (−)-α-pinene over (+)-α-pinene (as analysed using Multidimensional gas chromatography-mass spectrometry; MD GC-MS as described by Lücker et al 2002; Eur. J. Biochem 269: 3160-3171).

A range of other monoterpenes were also tested as substrates for the recombinant FaPINH protein and could also be hydroxylated at the position corresponding structurally to C10 in α-pinene (see FIG. 4). For example, the monocyclic terpenes (+)- and (−)-limonene were hydroxylated at C7 to yield perilla alcohol with only slightly lower efficiency than the hydroxylation of the bicyclic monoterpenes (+)- and (−)-α-pinene (FIGS. 3-4). In addition, structurally analogous substrates such as α-phellandrene, α-terpinolene and α-terpinene were hydroxylated at the same position (FIGS. 3 F,H,J; FIG. 4). In addition, the α-phellandrene, α-terpinene and limonene substrates used contained a trace impurity of p-cymene which was found to be hydroxylated also at C7 yielding 4-(1-methylethyl)-benzenemethanol (p-cymen-7-ol) (FIG. 4). Despite the reported high regio-selectivity of cytochrome P450 enzymes also some hydroxylation occurred in different regions. For limonene this second product peak could be identified as limonen-10-ol and for α-terpinolene also two product peaks were visible (in slower temperature program, not visible in FIG. 3) which were tentatively identified as the 7-OH and 10-OH alcohols (FIG. 4).

It was, thus, found that the P450 enzyme is able to hydroxylate a variety of terpene and/or terpene analogue substrates at two different positions, structurally corresponding to the C10 position of alpha-pinene, the C7 position of limonene and the C10 position of limonene as depicted in FIGS. 4 and 5. Although not all (putative) substrates have been tested yet, it is likely that the P450 enzyme according to the invention can also hydroxylate a range of other terpenoids at positions structurally corresponding to C9 or C10 in α-terpinolene and limonene, respectively: for example p-menth-8-ene and p-menth-4(8)-ene (FIG. 5). Or at the position corresponding structurally to C7 or C10 of limonene and α-pinene, respectively, for example p-menth-1-ene, γ-terpinene and 3-carene. Considering that also the aromatic monoterpene p-cymene is hydroxylated at the C7-carbon (analogous to limonene), it is very likely that other aromatic compounds can be hydroxylated at structurally analogous positions, for example aromatic hydrocarbons comprising a single aromatic ring, such as toluene to yield benzyl alcohol, a colorless liquid with weak, slightly sweet odour and constituent of many essential oils both free or as ester that is used in perfumery and flavour industries and as an anti-microbial preservative in pharmaceuticals and cosmetics.

Clearly, any of the immediate hydroxylation products catalyzed by PINH can be further modified (e.g. esterification, oxidation, glycosylation, etc.) by various means, such as in vivo in the host cell or organism (by recombinant or endogenous enzymes) or chemically. These further products are herein referred to as “derivatives” of PINH hydroxylation products. This includes either individual derivatives or mixtures of various derivatives. Such derivatives with useful properties, compositions comprising these and uses thereof (as well as methods of making these) are included in the invention. Especially, (mono)terpene alcohols may be modified to aldehydes and/or acids with biological activity (such as biocontrol activity).

For example, endogenous plant or additional engineered enzymes can further oxidise or otherwise modify the alcohols produced by PINH enzymes according to the invention and lead to additional interesting products such as the highly anti-microbial perilla aldehyde and perilla acid from perilla alcohol, the aphid-repellent myrtenal from myrtenol, limonene-10-al and/or compounds further derived from that which have interesting properties for the flavor and fragrance industry. Further modification of the immediate PINH hydroxylation products can include esterification, glycosylation, etc. For example it has been demonstrated for a number of plant species that transgenically introduced terpene alcohols will be glycosylated, e.g. completely in Petunia (Lücker et al., 2001. The Plant Journal 27: 315-324), and partly in Arabidopsis (Aharoni et al., 2003. The Plant Cell 15: 2866-2884) and potato (Jongsma, Aharoni, Bouwmeester et al., unpublished data). These glycosylated derivatives may, for example, serve as a slow-release source for improved protection against microbial and insect attack, or as a slow-release source in flavor and fragrance applications and products.

Cloning of the corresponding PINH gene from the wild species (termed FvPINH; see the protein depicted as SEQ ID NO: 5) showed that the proteins from the wild and the cultivated species differed by only three amino acid residues. FaPINH and FvPINH thus have an amino acid sequence identity of about 98.8%. Both proteins have a length of 520 amino acids and comprise an oxygen binding domain (amino acids 317-322; sequence AGTDTT) and a heme domain (amino acids 452-462; sequence PFGAGRRICPG). Amino acids 1-30 comprise a putative transmembrane domain for endoplasmatic reticulum anchorage.

The DNA sequences encoding FaPINH and PvPINH are depicted in SEQ ID NO: 2 and SEQ ID NO: 3, respectively. At the DNA level the sequence identity between the two coding regions is about 99.5%.

Northern blot analysis showed that the PINH gene is expressed in leaf, root and ripe fruit tissues of wild and cultivated species (FIG. 6). The results showed that PINH is expressed at high levels in ripe fruit of the wild species, higher than in ripe fruit of the cultivated species. Expression of PINH was detected in roots of both strawberry species (though at higher levels in the cultivated species), while only very low levels could be detected in leaves of both species (FIG. 6).

### Nucleic Acid Sequences and Proteins According to the Invention

In one embodiment of the invention nucleic acid sequences and amino acid sequences of PINH are provided, as well as methods for isolating or identifying functional orthologs of PINH in other species, such as (but not limited to) other plant species, e.g. other dicotyledonous or monocotyledonous species.

“PINH” proteins are defined by their amino acid sequence and, more importantly, their catalytic function. As shown in table 2, FaPINH and FvPINH have less than 50% amino acid identity to known sequences, while they have more than 90% identity to each other.

TABLE 2
amino acid sequence identity

| | CYP71A26 | CYP71A25 | CYP71A22 |
|---|---|---|---|
| FaPINH protein | 48.1% | 47.3% | 47.8% |
| FvPINH protein | 48.1% | 46.9% | 47.4% |

(GAP opening = 8, GAP extension = 2, Blosum62)

“PINH proteins” (or “PINH enzymes”) encompass all proteins with at least 20%, 30%, 35%, 40%, 45%, 50%, 60%, 70%, 80%, 90%, 95%, 98% or more sequence identity (as determined using pairwise alignment using the GAP program with a gap creation penalty of 8 and an extension penalty of 2) to SEQ ID NO: 4 and/or 5 and which are able to catalyze the hydroxylation of terpene or terpene analogue substrates, especially of monoterpene (or monoterpene analogue) substrates under suitable reaction conditions (i.e. they have “PINH enzymatic activity”, see below). Especially, functional PINH proteins are able to hydroxylate the C7 and/or C10 carbon (or C9 carbon) of (mono)terpene substrates (i.e. structurally analogous positions, see FIGS. 4 and 5) and/or the C7 position of aromatic hydrocarbons. Preferably PINH protein variants having some, e.g. 5-10, 20, 30 or more amino acids added (inserted), replaced or deleted without significantly changing the protein activity are included in this definition. For example conservative amino acid substitutions within the categories basic (e.g. Arg, His, Lys), acidic (e.g. Asp, Glu), nonpolar (e.g. Ala, Val, Trp, Leu, Ile, Pro, Met, Phe, Trp) or polar (e.g. Gly, Ser, Thr, Tyr, Cys, Asn, Gln) fall within the scope of the invention as long as the enzymatic activity of the PINH protein is not significantly, preferably not, changed, at least not changed in a negative way. In addition non-conservative amino acid substitutions fall within the scope of the invention as long as the activity of the PINH protein is not changed significantly, preferably not, or at least is not changed in a negative way. “PINH protein variants” encompasses all functional PINH fragments, mutated forms or modified proteins with the above amino acid sequence identity and with PINH enzymatic activity. The catalytic activity should be at least comparable to that of FaPINH or FvPINH, but may also be improved compared to these enzymes. Simple comparative activity assays can be carried out. Variants, and nucleic acid sequences encoding variants, can be either identified from natural sources or generated by mutagenesis, gene shuffling, de novo chemical synthesis, or other standard molecular biology techniques as known in the art. Functionality can be tested as described in Examples 3 and 6.

The PINH enzymes according to the invention are preferably highly regio-selective. Preferably at least 50, 60, 70, 80, 90, 95, 99 or 100% of the product is specifically hydroxylated at one specific carbon position, especially at the C10 or C7 position structurally analogous to the C10 or C7 position of α-pinene or limonene as depicted in FIGS. 4 and 5. Likewise, the enzyme is preferably highly substrate specific. “High specificity” refers to a specificity of at least 50, 60, 70, 80, 90, 95, 99 or 100%.

Also included are methods to modify the regio-selectivity of PINH enzymes using for example site directed mutagenesis methods known in the art. PINH enzymes with improved regio-specificity can be made, so that the enzyme only hydroxylates one specific carbon. For example the regio-selectivity for substrates such as limonene can be improved, so that the main product is the C7-hydroxylated product (perilla alcohol) and no or only trace amounts of the C10-hydroxylation product (limonene-10-ol) is produced. Alternatively, the region-selectivity for the C10 position may be enhanced. Similarly, the specificity for one of the enantiomers may be enhanced.

In accordance with the invention “PINH protein” also refers to any protein comprising the smallest biologically active fragment of SEQ ID NO's 4 and 5 and of any PINH proteins as defined above, which retains PINH enzymatic activity, i.e. the ability to terminally hydroxylate suitable terpene substrates and terpene analogous when contacted with those substrates under suitable reaction conditions. This includes hybrid and chimeric PINH proteins comprising the smallest active fragment. Preferably, at least one heme and one oxygen binding consensus domain is present, especially a heme domain or oxygen binding domain which is essentially similar or identical to that of SEQ ID NO: 5 and 6, i.e. amino acids 317-322 (sequence AGTDTT; oxygen binding) and a heme amino acids 452-462 (sequence PFGAGRRICPG; heme domain).

“PINH enzymatic activity” refers to the ability of the protein to catalyze the hydroxylation of the methyl group of terpene or analogues substrates such as aromatic compounds, especially the C10 hydroxylation of (mono)terpenes or terpene analogues having a structure similar to α-pinene and/or the C7 and/or C10 (or C9) hydroxylation of (mono)terpenes or terpene analogues having a structure similar to limonene, as depicted in FIGS. 4 and 5.

Suitable (monocyclic) monoterpene or aromatic substrates which may be C7 hydroxylated are: (+)-limonene and/or (−)-limonene, α-phellandrene, α-terpinene, γ-terpinene, terpinolene, p-cymene, p-menth-1-ene, 1,3,8-menthatriene and other isomers and analogues thereof (compounds derived from these). Further, suitable aromatic hydrocarbons which can be C7-hydroxylated are for example aromatic hydrocarbons comprising a single benzene ring, such as methylbenzene (toluene) to yield benzyl alcohol, or others.

Suitable (monocyclic) monoterpene substrates which may be C10 (or C9) hydroxylated are: (+)-limonene and/or (−)-limonene, α-terpinolene, p-menth-8-ene and p-menth-4(8)-ene and other isomers and analogues thereof (compounds derived from these).

Suitable (acyclic) monoterpene substrates which may be hydroxylated at the C6-methyl are ocimene, myrcene and other isomers and analogues thereof (compounds derived from these).

Suitable (acyclic) monoterpene substrates which may be C10 hydroxylated are ocimene and other isomers and analogues thereof (compounds derived from these).

Suitable (bicyclic) monoterpene substrates which may be C10 hydroxylated are: (+)-α-pinene and/or (−)-α-pinene (preferred), 3-carene and other isomers and analogues thereof (compounds derived from these).

In order to determine whether an enzyme has “PINH enzymatic activity”, an enzymatic activity assay comprising, for example, the following steps can be carried out:
contacting the (putative) PINH enzyme (or fragment or variant) with a suitable terpene- or terpene-analogue substrate, especially a substrate having a carbon skeleton structurally similar to that of (or identical to) α-pinene or limonene (as shown in FIGS. 4 and 5), under suitable reaction conditions, especially in the presence of a suitable electron donating protein and required co-factors, such as NADPH,
after a suitable reaction time analysing the presence of hydroxylated terpene or terpene analogue products by, for example, GC or GC-MS.

It is, therefore, one object of the invention to provide a method for determining whether a (recombinant) cell, tissue organism or a purified enzyme has PINH activity or whether a non-recombinant cell, tissue or organism has endogenous PINH activity, in order to isolate the responsible enzyme and/or gene. Catalytic activity and substrate specificity may also be compared to that of FaPINH or FvPINH in comparative assays.

When (−)- or (+)-α-pinene and (−)- or (+)-limonene are used as substrates the immediate oxidation products detected are (−)- or (+)-myrtenol and (−)- or (+)-perillyl alcohol, respectively. Obviously, if one or more additional enzymes are present, which are able to further react with the hydroxylated product, the amount of the expected product may be substantially reduced, and other products may be detectable. For example the OH group at the C10 position of myrtenol may be further oxidised by for example dehydrogenases leading to the corresponding aldehyde, and/or esterified by an alcohol acyl transferase (AAT) to produce myrtenyl acetate and/or other esters. Similarly, perillyl alcohol may be oxidised or esterified to form for example perillic aldehyde, perillic acid, perillyl acetate and/or other esters, respectively.

The enzyme activity assay used may be an in vitro or an in vivo assays. “Contacting” refers therefore either to the in vivo contact between enzyme and substrate in a cellular compartment, or the in vitro contact in a medium (cell free or comprising a cell lysate or fraction). For example, the DNA sequence encoding the PINH enzyme may be cloned into an expression vector and used to (stably or transiently) transform a suitable host cell. The in vivo conversion of a suitable substrate (either supplied to the cells with the growth media or produced by the host cell) to the hydroxylated product can then be analysed. For example, if the host cell is a recombinant plant cell, explants (e.g. a leaf) may be supplied with medium comprising the substrate and the resulting presence and quantity of PINH hydroxylation products may be determined. Similarly, the (recombinant) host cell lysate or cell fraction (e.g. the ground tissue or microsomal fraction comprising the membrane bound PINH enzyme) may be contacted with a suitable substrate by adding suitable amounts of substrate, co-enzymes and co-factors to the fraction. In in vivo systems hosts cells may be cell cultures or tissues (e.g. plant tissue for transient PINH expression) or whole organism. Enzymatic activity assays and methods for producing terminally hydroxylated terpenes or terpene analogues are further described elsewhere herein in more detail.

“Suitable reaction conditions” depend on whether a host cell, host cell fraction/lysate or (partially) purified (or synthetic) enzyme is used and, if a host cells is used, the type of host cell, but in general the term refers to the in vivo or in vitro microenvironment which allows the PINH enzyme to fold properly and to have catalytic activity. Especially, the presence of a functional, co-localized electron donating protein is necessary, as well as the presence of suitable co-factors such as NADPH.

If the host cell is a plant cell, the endogenous plant electron donating protein (especially the endogenous NADPH-cytochrome P450 reductase; EC 1.6.2.4) is capable of efficient electron transfer (see Lücker et al. Plant J. 2004 July; 39(1): 135-45 and Urban et al 1997, J. Biol. Chem. 272, 19176-19186, showing that NADPH reductases have the ability to function as electron donors for heterologous cytochrome P450 enzymes). For plant host cells, or plant cell-membrane comprising extracts a suitable NADPH cytochrome P450 reductase (CPR) is inherently available. Optionally, a chimeric gene encoding a (homologous or heterologous) CPR may be co-expressed in the host cell to provide or enhance this function.

For use of host cells which do not contain an endogenous CPR, such as bacterial hosts, or which contain an endogenous CPR which does not enable high catalytic activity of the PINH enzyme, such as yeast cells (whose endogenous CPR does function as electron donor, but not very efficiently), the PINH enzyme must either be purified and reconstituted with a CPR in a membrane-like environment (e.g. in di-lauryl-phosphatidylcholine micelles as described by Bak et al. 1997, Plant J. 11:191-201, Bak et al. 1998, Plant Mol Biol 36: 393-405 or WO01/51622) or the host cell must be modified to produce a functional CPR. For yeast, modified strains are already available which either overexpress the endogenous yeast CPR (e.g. Saccharomyces cerevisiae strain W(R)) or which overexpress Arabidopsis thaliana CPR genes (ATR1 or ATR2), whereby the Arabidopsis CPR gene replaces the endogenous yeast CPR1 gene (e.g. Saccharomyces cerevisiae strains WAT11U and WAT21U), see Pompon et al. 1996 (Method. Enzymology 272, 51-64), Urban et al. 1997 (J. Biol. Chem. 272, 19176-19186). Similarly, insect cells expressing endogenous insect CPR is suitable for functional expression of plant cytochrome P450 enzymes (O'Reilly et al. 1992, Baculovirus expression vectors: A laboratory manual; New York, Freeman, 364). The electron donor may, thus, be provided by choosing an appropriate host cell or by modifying the host cell accordingly, as will be further described below.

PINH enzymes as defined above, and nucleic acids encoding these, may be isolated from other prokaryotic or eukaryotic species as known in the art. For example in silico analysis may be carried out using any one of SEQ ID NO: 1 to 5 in order to identify putative PINH enzymes in nucleic acid or protein databases (e.g. GENBANK, SWISSPROT, TrEMBL) using standard sequence analysis software, such as sequence similarity search tools (BLASTN, BLASTP, BLASTX, TBLAST, FASTA, etc.). Subsequently the catalytic function then needs to be tested, using an in vivo or in vitro enzymatic activity assay, and optionally compared to that of FaPINH and/or FvPINH. For this purpose the sequences may be cloned or synthesized de novo and tested for its enzymatic activity. Also, analysis of the terpenes and terpene analogous found in organisms, e.g. in the ripe fruits or other tissues of plants (pine needles, leaves, roots, etc), may already provide an indication of the presence of a PINH enzyme and may be used as a selection criterion to identify organisms which comprise a functional PINH enzyme.

Alternatively, specific or degenerate primers or probes based on SEQ ID NO: 1 to 3 may be made using known methods and used to amplify or hybridize to DNA or RNA isolated from other species, especially from species identified to contain monoterpenoids or monoterpene analogs oxidised at the relevant methyl group such as Rubus spp., other Fiagaria spp, Perilla frutescens, Asteriscus maritimus, Nepeta spp, Amomum testaceum, Syzygium spp., walnut, Hyssopus officinalis, Lippia multiflora, Xylopia aromatica, Geum spp., or any other (wild or cultivated) plant species containing products obviously produced by C10 (or C9) and/or C7 hydroxylation, for example any of the products shown in FIGS. 4 and 5. In particular, (other) members of the Rosaceae, Asteraceae, Lamiaceae and aromatic tree species, such as Pinus species may also be suitable.

In the same way PINH enzymes and DNA encoding these may be isolated from other organisms, such as fungi, bacteria or animals. Suitable candidates are species which produce C10 (or C9) and/or C7 hydroxylated and/or further oxidised monoterpenes such as myrtenol or myrtenol derivatives (e.g. myrtenyl acetate) and/or are able to (bio)convert externally supplied monoterpenes or monoterpene analogs to C10 and/or C7 hydroxylated and/or further oxidised products, such as Hansenula capsulata and Candida nitratophila (Leufven et al., 1988. J Chem Ecol 14: 353-362).

PINH proteins according to the invention may be isolated from natural sources, synthesized de novo by chemical synthesis (using e.g. a peptide synthesizer such as supplied by Applied Biosystems) or produced by recombinant host cells. The PINH proteins according to the invention may be used to raise mono- or polyclonal antibodies, which may for example be used for the detection/isolation of PINH proteins in/from samples (immunochemical analysis methods and kits).

Also provided are nucleic acid sequences (genomic DNA, cDNA, RNA) encoding PINH proteins, as defined above (including any chimeric or hybrid PINH proteins. Due to the degeneracy of the genetic code various nucleic acid sequences may encode the same amino acid sequence. SEQ ID NO: 1 to 3 depict the PINH cDNA and coding sequences from Fragaria x ananassa and Fragaria vesca. The nucleic acid sequences provided include naturally occurring, artificial or synthetic nucleic acid sequences. Included are also sequences generated from the provided sequences by e.g. gene shuffling methods as described in U.S. Pat. No. 5,811,238, WO97/20078, U.S. Pat. No. 6,180,406 and U.S. Pat. No. 6,117,679, which encode PINH proteins comprising higher or modified catalytic activity and methods of using the nucleic acid sequences of the invention for generating such “evolved” sequences. It is understood that when sequences are depicted as DNA sequences while RNA is referred to, the actual base sequence of the RNA molecule is identical with the difference that thymine (T) is replace by uracil (U).

Also included are variants and fragments of PINH nucleic acid sequences, such as nucleic acid sequences hybridizing to PINH nucleic acid sequences under stringent hybridization conditions as defined. Variants of PINH nucleic acid sequences also include nucleic acid sequences which have a sequence identity to SEQ ID NO: 1, 2 or 3 of at least 30%, 40%, 50%, 60%, 70%, 80%, 90%, 95, 99% or more.

It is clear that many methods can be used to identify, synthesise or isolate variants or fragments of PINH nucleic acid sequences, such as nucleic acid hybridization, PCR technology, in silico analysis and nucleic acid synthesis, and the like.

The nucleic acid sequence, particularly DNA sequence, encoding the PINH proteins of this invention can be inserted in expression vectors to produce active PINH enzyme, as described below, and especially to produce the desired C10 (or C9) and/or C7 hydroxylation product or C7 hydroxylated aromatic hydrocarbons and/or further derivatives of any of these. For optimal expression in a host or host cell the PINH DNA sequences can be codon-optimized by adapting the codon usage to that most preferred in the host cell. Codon usage tables are publicly available, see e.g. http://www.kazusa.or.jp/codon/. When the host cell is a plant cell the codon usage may be adapted to plant genes native to the plant genus or species of interest (Bennetzen & Hall, 1982, J. Biol. Chem. 257, 3026-3031; Itakura et al., 1977 Science 198, 1056-1063.) using available codon usage tables (e.g. more adapted towards expression in cotton, soybean corn or rice). Codon usage tables for various plant species are published for example by Ikemura (1993, In “Plant Molecular Biology Labfax”, Croy, ed., Bios Scientific Publishers Ltd.) and Nakamura et al. (2000, Nucl. Acids Res. 28, 292.) and for various organisms in the major DNA sequence databases (e.g. EMBL at Heidelberg, Germany). Accordingly, synthetic DNA sequences can be constructed so that the same or substantially the same proteins are produced. Several techniques for modifying the codon usage to that preferred by the host cells can be found in patent and scientific literature. The exact method of codon usage modification is not critical for this invention. Especially for expression of plant derived PINH genes in yeast, fungi, bacteria, insects or mammalian cells, codon-usage may be (partially) adapted to that of the host cell (see Batard et al. 2000, Arch Biochem Biophys 379, 161-169). Likewise, codon usage of a monocot derived PINH may be (partially) adapted to a dicot preferred codon usage for expression in dicots, and vice versa (see Batard et al. 2000, supra).

Small modifications to a DNA sequence such as described above can be routinely made, i.e., by PCR-mediated mutagenesis (Ho et al., 1989, Gene 77, 51-59., White et al., 1989, Trends in Genet. 5, 185-189). More profound modifications to a DNA sequence can be routinely done by de novo DNA synthesis of a desired coding region using available techniques.

Also, the PINH nucleic acid sequences can be modified so that the N-terminus of the PINH protein has an optimum translation initiation context, by adding or deleting one or more amino acids at the N-terminal end of the protein. Often it is preferred that the proteins of the invention to be expressed in plants cells start with a Met-Asp or Met-Ala dipeptide for optimal translation initiation. An Asp or Ala codon may thus be inserted following the existing Met, or the second codon can be replaced by a codon for Asp (GAT or GAC) or Ala (GCT, GCC, GCA or GCG). The DNA sequences may also be modified to remove illegitimate splice sites.

For expression in prokaryotic host cells, such as E. coli, it has been found that N-terminal modifications are necessary to enable optimal functional expression. Especially, the native N-terminal amino acid sequence (i.e. comprising the putative ER membrane anchor of the PINH enzyme) may need to be removed or truncated, modified or extended. For example, Halkier et al. (1995, Arch Biochem Biophys 322:369-377; WO01/51622) were able to optimize expression by reducing the length of the N-terminal hydrophobic core of the cytochrome P450 targeting sequence and by exchanging the first 8 codons with the first eight codons of bovine P45017 alpha. See also Bak et al. 1997, supra. Thus, the putative ER anchor comprised in amino acids 1-30 of SEQ ID NO: 4 and 5 may be removed, truncated, modified or replaced for optimal expression.

In one embodiment of the invention PCR primers and/or probes and kits for detecting the PINH DNA sequences are provided. Degenerate or specific PCR primer pairs to amplify PINH DNA from samples can be synthesized based on SEQ ID NO's 1-3 as known in the art (see Dieffenbach and Dveksler (1995) PCR Primer: A Laboratory Manual, Cold Spring Harbor Laboratory Press, and McPherson at al. (2000) PCR-Basics: From Background to Bench, First Edition, Springer Verlag, Germany). Likewise, DNA fragments of SEQ ID NO's 1-3 can be used as hybridization probes. A PINH detection kit may comprise either PINH specific primers and/or PINH specific probes, and an associated protocol to use the primers or probe to detect PINH DNA in a sample. Such a detection kit may, for example, be used to determine, whether a plant has been transformed with a PINH gene (or part thereof) of the invention. Because of the degeneracy of the genetic code, some amino acid codons can be replaced by others without changing the amino acid sequence of the protein. Also, antibodies that bind specifically to a PINH protein according to the invention are provided.

### Chimeric Genes Vectors and Recombinant Cells/Organisms According to the Invention

In one embodiment of the invention nucleic acid sequences encoding PINH proteins, as described above, are used to make chimeric genes, and vectors comprising these for transfer of the chimeric gene into a host cell and production of the PINH protein(s) in host cells, such as cells, tissues, organs or organisms derived from transformed cell(s). Host cells are selected from plant cells, microbial hosts (bacteria, yeast, fungi, etc.), viruses and animal cells (insect cells, mammalian cells, human cells, etc.). The choice of host depends on the ultimate use of the hydroxylated (mono)terpene or aromatic product (or one or more further derivatives of these), which is to be produced. In one embodiment the vectors are gene silencing vectors comprising sense and/or antisense nucleic acid sequences of PINH genes. Below the uses of PINH genes according to the invention are first briefly summarized and further below explained in more detail:

1. In one embodiment methods for generating a plant (or certain plant tissue) with modified properties are provided, especially with modified taste and/or fragrance (i.e. aroma) properties of edible (e.g. fruit, leaves, nuts, seeds, roots, tubers, stems, etc.) and/or non-edible parts (e.g. parts used for decorative purposes, such as cut flowers) of the recombinant plant, or the whole plant. This is especially done by overexpressing a PINH coding sequence according to the invention within the host cell(s). Optionally, a chimeric gene encoding an electron donating protein (especially an NADPH-cytochrome P450 reductase) is also transferred and expressed in the host cell in order to enhance the PINH activity. Depending on the desired product, the nature of the edible and/or non-edible part(s) and depending on the natural PINH substrate availability within the plant (or plant tissue), it may be necessary to introduce additional genes into the plant which are (a) able to generate the required monoterpene substrate used by PINH (for example terpene synthase, especially monoterpene synthase enzymes, such as described in WO02/064764 and/or (b) which are able to further react with the hydroxylated monoterpene product generated by PINH oxidation (for example AAT enzymes may be expressed). The plant or plant tissue comprising the PINH enzyme and modified taste and/or fragrance may be used directly (the tissue may be consumed as such or used for decoration) or may be further processed to food/feed or cosmetic compositions (solid, semi-solid or liquid). Alternatively, the hydroxylated terpene or terpene analogue products may be further purified from the tissue and used as flavouring agent and/or fragrance agent in the preparation of other compositions. In another embodiment the flavour and/or fragrance is modified by silencing the endogenous PINH gene(s) or gene family. As some products of PINH catalysis and/or further derivatives thereof, such as for example myrtenol or perillyl alcohol and their derivatives, have a role in flavour/fragrance as well as in bio-control (point 2), a plant or plant tissue may have both a modified flavour/fragrance and enhanced bio-control properties. In another embodiment host cells, tissues, organs or organism having anti-carcinogenetic properties due to the in vivo production and accumulation of perillyl alcohol are provided, as well as food/feed compositions with anti-carcinogenic properties (see point 4). When the host cell is a plant cell, this trait may be combined with modified fragrance and/or flavour (point 1) and/or enhanced bio-control properties (point 2).

2. As many terpenes and terpene analogous have bio-control activity, one embodiment encompasses the generation of recombinant host cells, especially plants, plant tissues, or recombinant microorganisms, with enhanced bio-control properties, especially disease and/or pest resistance. For example production of plant tissues with constitutive or inducible myrtenol production and/or perilla alcohol production (and/or any further derivatives of these, such as myrtenal, perilla aldehyde and/or perilla acid) is one aspect of the invention. Another aspect is the production of microorganisms with bio-control properties. The recombinant plant or microorganism may be used itself, compositions comprising the microorganism may be made, or the recombinant host cells may be further processed to extract or purify the hydroxylated terpene or terpene analogous (or further derivatives) which may then be used as bio-control agent and in the preparation of bio-control compositions.

3. Some insect species are attracted by terpenes or terpene analogues. In one embodiment recombinant host cells themselves or the terpenes/analogues (or derivatives) produced by such cells are used as insect attractants. Also, RNAi strategies are used to change the insect-attractant properties of plants, by silencing the endogenous PINH gene(s) or gene family. Such plants lose (or reduce) their insect attractant properties and are thus protected from damage caused by the insects. Also provided are methods for trapping pests and pest-traps. It is understood that plants overexpressing PINH in certain tissue, while silencing endogenous PINH in other tissues are included herein. For example, overexpression in root tissues may provide modified fragrance/flavour, while silencing in aerial tissues may provide reduced insect attraction.

4. In another embodiment the recombinant host cell is used as a factory to manufacture a desired hydroxylated (mono)terpene and/or aromatic hydrocarbon by PINH activity (optionally in combination with other recombinant enzymes or externally applied substrates) or one or more further derivatives of the immediate PINH product. The “recombinant” end-product in these applications is the substantially purified reaction product and various compositions comprising the product in suitable amounts. The product may then be used for various purposes. Desired products are especially (+) and/or (−) perillyl alcohol, which is a compound having anti-carcinogenic properties and which is difficult to produce by known methods. Other desired products may be any of the products shown in FIGS. 4 and 5, or further derivatives thereof. For such applications a microbial host cell (e.g. bacteria, yeast or other fungi) may be preferred to plant or animal host cells, as these can more easily be upscaled. However, recombinant plant or animal cell cultures (e.g. insect cells, such as Sf9 cells or mammalian cells such as HT-1080 cells, or NSO cells) or whole organisms are also suitable. Provided are also various compositions and uses of the hydroxylated product in the food/feed industry, as a pharmaceutical composition or as a bio-control agent. In addition, the recombinant cells, tissues or organisms may themselves be used as anti-carcinogenic food or feed or may be used to manufacture food/feed compositions having anti-carcinogenic properties. This method does not require expensive and time consuming purification of the hydroxylation products. Preferred products are for example tobacco plants or leaves producing and accumulating perillyl alcohol, whereby cigarettes manufactured from such plants have a therapeutic and/or prophylactic anti-carcinogenic effect (e.g. the risk of developing lung cancer is significantly less than when an equivalent amount of traditional cigarettes are smoked). Other health beneficial, both prophylactic and therapeutic, uses are envisaged, e.g. edible fruit, vegetables or other cells/tissues, and compositions comprising these, with anti-carcinogenic properties when consumed.

5. In yet another embodiment the PINH coding sequence is used in gene therapy approaches, in order to directly produce perillyl alcohol in target cells in humans and/or animal subjects. In this approach one or more coding sequences are expressed in the target cell (e.g. the cancer cell), such as at least the PINH coding sequence and optionally one or more sequences encoding enzymes which lie upstream of PINH, e.g. a monoterpene synthase (such as a limonene synthase) and a geranyl diphosphate synthase (GPP synthase). The substrate limonene may also be provided to the target cells orally or by injection such that only PINH is required.

It is noted that although different embodiments of the invention are separated into different sections under different headings, it is clear that there is considerable overlap and that especially general aspects, such as vectors, transformation methods, suitable hosts, suitable substrates, compositions, methods and uses etc. are generally applicable throughout and also apply to embodiments described herein under a different heading.

1. Recombinant Plants or Plant Tissues with Modified Terpene or Terpene Analogue Profiles (or Further Derivatives Thereof) Comprising Modified Flavour and/or Fragrance

### 1.1 Chimeric Genes, Vectors and Recombinant Host Cells

In one embodiment a PINH encoding sequence is used to transform a plant cell, in order to generate plants or plant organs (e.g. fruit) with modified taste and/or fragrance properties. The term “fruit” is used herein either in the botanical sense (the ripened ovary and its content) or in the common sense, whereby aggregate fruit (e.g. strawberry, wherein the small fruit are combined on a receptacle) and multiple fruit are encompassed. The botanical meaning of fruit also encompasses what is commonly known as vegetables, grains, nuts, etc.

By (over)expressing the gene encoding PINH (for example SEQ ID NO: 2 or 3) in a plant cell, specific tissue(s) or in the whole plant, it is possible to significantly alter the terpene or terpene analogue profile (the total and the relative amounts) of the cells, tissue(s) or plant. This in turn will have an effect on the flavour and/or fragrance, and the overall aroma, of the tissue or plant. In this way for example fruits with modified flavour and/or fragrance may be generated or ornamental plants (e.g. flowers or house plants) with modified fragrance may be produced. In one embodiment the PINH coding sequence is used to produce C10 (or C9) and/or C7 hydroxylated monoterpenes (see FIGS. 4 and 5) in vivo in plant cells, tissues, organs or whole plants, or further derivatives of the immediate hydroxylation product. The method comprises the steps of: a) transforming a plant cell with a chimeric gene comprising a nucleic acid sequence encoding a functional PINH enzyme, wherein the nucleic acid sequence is operably linked to a (constitutive, inducible, tissue specific or developmentally regulated) promoter active in plant cells, b) regenerating and selecting plants which express the chimeric gene, whereby the cells in which the PINH enzyme is produced have a modified flavour and/or fragrance when sufficient amounts of a suitable PINH substrate is contacted with the enzyme (the substrate being either produced in vivo or supplied externally to the cells, tissue or plant). Optionally, additionally one or more chimeric genes encoding PINH substrate producing enzymes and/or PINH product modifying enzymes (as defined below) are introduced into the plant cell under the control of suitable promoters. Optionally, a chimeric gene encoding an NADPH-cytochrome P450 reductase is also introduced into the cell, in order to enhance PINH activity.

The nucleic acid sequences may be co-transformed into one cell, consecutively transformed (by transforming an already transformed cell) or combined in one plant by crossing separately transformed plants, as described further below.

For example, expression of PINH in cultivated strawberry in combination with FvPINS alters the taste and/or fragrance of the recombinant strawberry in such a way that it resemble wild strawberry more closely. As cultivated strawberry comprises a functional alcohol acyl transferase enzyme some or all of the myrtenol will be esterified in tissues where the AAT enzymes is co-expressed (e.g. during fruit ripening), thereby producing esters, such as the volatile myrtenyl acetate. Thus, both the enhanced amount of myrtenol and/or the enhanced amounts of derivatives of myrtenol alter the flavour and/or fragrance of the tissue.

As myrtenyl derivatives, such as myrtenyl acetate, could have more desirable flavour and/or fragrance properties than myrtenol, it is desired to enable contact of the myrtenol produced in vivo with an alcohol acyl transferase (AAT) enzyme, in order to convert some or all of the myrtenol into myrtenyl derivatives, such as myrtenyl acetate. Clearly, if the host cells in which the myrtenol is produced already naturally produce a functional AAT enzyme, it may not be necessary to introduce a chimeric gene comprising an AAT coding sequence, depending on the expression pattern (tissue and time) of AAT gene. However, if a functional AAT enzyme is missing in the host cell or if expression of the endogenous AAT gene does not coincide with the expression pattern of the PINH transgene, it is an embodiment of the invention to additionally introduce a chimeric gene encoding a functional AAT enzyme into the genome of the same host cell(s). For example, both the PINH gene and the AAT gene may be operably linked to constitutive promoters, to inducible promoters or to promoters substantially overlapping in their expression pattern.

Similarly chimeric genes comprising coding sequences of other “PINH-product modifying enzymes” may be introduced into the host cell, such as for example alcohol dehydrogenases, other cytochrome P450s and glycosyl transferases. Such DNA and protein sequences are widely available or can be cloned using routine methods.

Suitable AAT coding sequences are widely available in the art or can be cloned using known methods. For example WO00/32789, Beekwilder et al. 2004 (Plant Physiol 135: 1865-1878) and Aharoni et al. 2000 (supra) disclose alcohol acyl transferase cDNAs from various plant species, such as melon, wild and cultivated strawberry, tomato, banana, apple, mango and lemon. Especially AAT enzymes that have a high substrate specificity for terpene alcohols, especially myrtenol, are suitably used. A skilled person can easily determine the substrate specificity using known methods, such as in vitro substrate preference assays or enzyme activity assays with plant material (both as e.g. described on page 1876 and 1877 of Beekwilder et al. 2004, supra). The AAT gene used may be heterologous or homologous to the plant species into which is introduced.

The above also applies to the availability of the PINH substrate. In host cells where a suitable PINH substrate, for example (+) and/or (−) α-pinene, is not produced, or is not produced in sufficient amounts (or not at the right time/location), it may be desirable to additionally introduce one or more genes encoding “PINH substrate-producing enzymes”. For example, a terpene synthase (e.g. a monoterpene synthase such as α-pinene synthase or limonene synthase) and optionally also a GPP synthase may be introduced under the control of a suitable promoter and in the appropriate cellular compartment. A recombinant host cell, tissue or a recombinant plant may thus comprise either a PINH encoding gene alone- or in combination with one or more additional transgenes encoding enzymes which lead to PINH substrate production and/or enzymes which further bio-convert the hydroxylated PINH product. Thus, in one embodiment the recombinant host cell, tissue or plant comprises a chimeric gene encoding a functional PINH enzyme together with a chimeric gene encoding a functional “PINH-product modifying enzyme” (e.g. an AAT enzyme) and/or one or more “PINH substrate-producing enzymes”. The change in flavour and/or fragrance will vary depending on the enzyme combinations and host cells used. The desired combinations can be determined using routine trial and error.

The term “PINH substrate-producing enzyme(s)” is used herein to refer to enzymes which catalyze reactions that lead to the production of (mono)terpenes or analogous thereof (or aromatic hydrocarbons) which can serve as substrates for PINH, i.e. which have a carbon skeleton structure essentially similar to that of limonene or α-pinene (see above, and FIGS. 4 and 5.). Such enzymes include: GPP synthases, limonene synthases, α-pinene synthases, α-phellandrene synthases, α-terpinene synthases, α-terpinolene synthases, 3-carene synthases, etc. Genes encoding PINH substrate producing enzymes have been or can be cloned from many species and are available to a skilled person.

Suitable GPP synthase coding sequences are for example described in U.S. Pat. No. 5,876,964 (from Mentha spp.), in U.S. Pat. No. 6,395,525, U.S. Pat. No. 6,303,330 and in Tholl et al. 2004 (Plant Cell 16 (4), 977-992; GPP synthase from Antirrhinum majus and Clarkia breweri). They have also been cloned from a range of other plant species such as Vitis vinifera (AY351862), Arabidopsis (NP_{—}173148), Abies grande (Burke and Croteau, Arch. Biochem. Biophys. 405: 130), Citrus (Bouvier et al. 2000, Plant J. 24: 241-252), Taxus (Hefner et al. 1998, Arch. Biochem. Biophys. 360: 62-74) etc.

Suitable (+) and/or (−) limonene synthases have been cloned from various plant species, such as Citrus limon (Lucker et al. 2002, Eur. J. Biochem. 269: 3160-3171), Citrus unshiu, Abies grandis (Bohlmann et al. 1997, J Biol Chem 272:21784-21792; Trapp and Croteau 2001, Genetics 158:811-832; Bohlmann et al. 1999, Arch Biochem 368:232-243), Chamaecyparis obtuse (hinoli cypress), Perilla frutescens, Schizonepefa tenuifolia, Mentha spicata (spearmint; Colby et al. 1993, J. Biol. Chemistry, 268:23016-23024; U.S. Pat. No. 5,871,988), Perilla citriodora (AF241790), Mentha longifolia (AF175323), and others.

Suitable genes encoding (+) and/or (−) α-pinene synthases are also available in the art. They have been cloned for example from strawberry (WO02/064764), Pinus taeda. (Phillips et al., 2003, Arch. Biochem. Biophys. 411: 267-276), Picea (AY237645), etc.

Also, enzymes and their corresponding genes from other organisms, such as for example alcohol dehydrogenases from fungi or other organisms, can be suitable.

The transgene(s) are preferably stably integrated within the host genome. The recombinant cells or tissues or plants can be easily distinguished by the presence of the recombinant DNA (detectable by PCR-based methods, nucleic acid hybridization based methods, etc.) or the RNA transcript levels (using e.g. quantitative RT-PCR), and by a modified terpene or terpene-analogue composition (or further derivatives thereof), especially by a significantly increased amount of C7 and/or C10-hydroxylated terpene or terpene analogous products (e.g. myrtenol) and/or products derived therefrom by further enzymatic modification (e.g. myrtenyl acetate or glycosides). A “significantly increased amount” or “significantly enhanced amount” or an “enhanced level” of a hydroxylation product or further derivative refers to an increase of at least 0.1%, 0.5%, 1%, 2%, 3%, 4%, 5%, 10%, 20%, 30%, 40%, 50%, 70%, 90% or 100% compared to the control cells/tissues/plants (e.g. cells transformed with a control construct or non-transformed cells of the same genetic background). Enhanced levels are thus levels above those present in the control cells/tissue, especially in an amount which is statistically significant compared to the control. A small increase in the amount may already be significant and may have a profound effect on the characteristics of the cell, tissue or organism, e.g. the flavour and/or odor may be modified. This definition applies throughout the specification. The increase can be easily determined by analysing the terpene profiles of the cells or tissues (whereby the terpenes or terpene analogues are extracted from the tissue using for example an organic solvent or SPME, i.e. solid phase microextraction), or the volatile compounds emitted from tissue samples or whole plants and trapped (using e.g. a Tenax cartridge). Both the volatiles present in a solvent and the volatiles emitted can be analysed using known methods such as GC-MS or other known methods. Glycosylated products such as myrtenol can be detected using LC-MS or GC-MS after (enzymatic) hydrolysis. In one embodiment myrtenol is significantly increased (either as free form or as glycosylated form) and/or myrtenol derivatives, such as myrtenyl acetate, myrtenal or myrtenilic acid are significantly increased in the recombinant cells, tissue or plant. In another embodiment myrtenol is further modified by an endogenous or recombinant enzyme to myrtenal which has good biocontrol properties (see elsewhere herein). In another embodiment myrtenol is glycosylated by an endogenous or recombinant enzyme to myrtenyl glycoside which can act as a slow release source for myrtenol.

If the PINH substrate producing enzyme is a limonene synthase, perilla alcohol will be produced from limonene by the action of PINH. Perilla alcohol has strong anti-cancer activity and plants expressing this compound in high levels may be used for therapy or to extract perilla alcohol for manufacturing medicine (see elsewhere herein).

The additional C10 hydroxylation found for limonene, leading to limonene-10-ol, is interesting for the flavor and fragrance industry. In one embodiment host cells, especially plants, tissues or organs comprising significantly increased amounts of perilla alcohol and/or limonene-10-ol, and/or one or more further derivatives of these are provided (see elsewhere herein). Limonen-10-ol, -10-al and dihydrolimonene-10-al have interesting flavour and/or fragrance properties and are difficult to obtain using known methods. Transgenic plants expressing PINH and a (endogenous or recombinant) limonene synthase produce limonen-10-ol which can be used as substrate by endogenous plant enzymes or be extracted and used for enzymatic or semi-synthetic production of these compounds.

In one embodiment myrtenol and/or myrtenol derivative (e.g. myrtenyl acetate) levels are significantly increased in edible parts of the host plant, such as fruits or leaves (e.g. lettuce, brussel sprouts), roots (e.g. ginger), flower heads (e.g. broccoli, cauliflower), seeds (e.g. nuts) or tubers (e.g. potato). For example, bananas, strawberries, mangos, peppers, nuts, corn, grape, lemon, grapefruit, orange, kiwi, and any other commonly eaten fruit or seeds (including vegetables, cereals, nuts) with modified flavour and/or fragrance due to a significantly enhanced level of myrtenol and/or one or more myrtenol derivatives is a preferred embodiment of the invention. In another preferred embodiment myrtenol and/or one or more myrtenol derivatives are significantly increased in non-edible (or not commonly eaten) parts, such as the flowers of plants, especially in ornamental plants, such as tulip, chrysanthemum, rose, etc.

Apart from changing the relative levels of myrtenol and/or one or more myrtenol derivatives it is also part of the invention to change the overall level of these compounds and to introduce the production of myrtenol and/or myrtenol derivatives into cells, tissues or plants which naturally do not produce these compounds at all or only at very low levels. In certain embodiments the total and/or relative amounts of myrtenol and/or myrtenol derivatives are changed in essentially all parts of the recombinant plant (by constitutive expression). However, in other embodiments specific production in one or more tissues or organs (e.g. in fruit only, in roots, flowers or in photosynthetically active tissues) is desired, and may be achieved by choosing appropriate tissue- or developmentally regulated promoters, as described below. Tissue specific expression generally places a lower metabolic burden on the plant. In a further embodiment PINH substrate is supplied to the recombinant plant cells, tissues or organs externally. Thus, the substrate may be sprayed onto the tissue, added to the soil or water, added to cut parts of the plant, e.g. to the water of cut flowers, etc. This method allows external control of the PINH activity and thereby induces or enhances the change in flavour and/or fragrance following the supply of substrate. The method comprises the steps of contacting the recombinant plant cells, tissues or plant with a suitable amount of PINH substrate, especially α-pinene. Substrates are commercially available (e.g. from Sigma-Aldrich) or may be produced chemically or recombinantly. The PINH promoter used may also be chemically inducible, e.g. substrate induced.

It is needless to mention that, when several genes of a biosynthetic pathway are introduced into the same host (e.g. PINH in combination with GPP synthase and/or α-pinene synthase, and/or AAT), it is necessary to ensure that the subcellular localization of the enzymes is such that the contact between the respective substrates and enzymes is possible. Thus, targeting sequences may need to be added or deleted, depending on the transformation method used (integration into the nuclear or plastid genome) and the desired compartment where the C10 and/or C7 hydroxylated monoterpene product is to be produced (e.g. in the cytoplasm, within a vacuole, secreted into the extracellular space, etc). Terpene synthases usually comprise an N-terminal plastid targeting peptide, while cytochrome P450 enzymes comprise an N-terminal ER targeting peptide. The retention of these natural targeting signals, or the replacement with equivalent signals, may thus be desired to achieve appropriate in vivo functionality (see e.g. Lücker et al. 2004, The Plant Journal 39:135). Additionally, the choice of promoters needs to be such that there is a sufficient tempo-spacial overlap in the expression profiles. A skilled person can easily chose appropriate promoters without undue experimentation.

Co-expression of PINH and other coding sequences in the same cell(s) can be achieved using known methods (see e.g. Lücker et al 2004, supra). For example by further transforming a plant already expressing a recombinant PINH protein, or by crossing plants transformed individually with chimeric genes, such as NADPH-cytochrome P450 reductase, PINH, AAT, GPP synthase and/or terpene synthases. Alternatively, the PINH coding sequence and the nucleic acid sequences encoding one or more other enzymes can be present on a single DNA vector or be co-transformed at the same time using separate vectors and selecting transformants comprising two or more chimeric genes. Another possible method is to generate a bicistronic or multicistronic nucleic acid sequence, allowing expression of two, three or more coding sequences from a single transcript using IRES elements (internal ribosome entry sites) see e.g. Hsieh et al. 1995 (Biochemical Biophys. Res. Commun. 214:910-917) or Fux et al. 2004 (Biotechnol Bioeng. April 20; 86(2):174-87).

In principle, any plant may be a suitable host, such as monocotyledonous plants or dicotyledonous plants, for example maize/corn (Zea species), wheat (Triticum species), barley (e.g. Hordeum vulgare), oat (e.g. Avena sativa), sorghum (Sorghum bicolor), rye (Secale cereale), soybean (Glycine spp, e.g. G. max), cotton (Gossypium species, e.g. G. hirsutum, G. barbadense), Brassica spp. (e.g. B. napus, B. juncea, B. oleracea, B. rapa, etc), sunflower (Helianthus annus), safflower, yam, cassava, tobacco (Nicotiana species), alfalfa (Medicago sativa), rice (Oryza species, e.g. O. sativa indica cultivar-group or japonica cultivar-group), forage grasses, pearl millet (Pennisetum spp. e.g. P. glaucum), tree species (Pinus, poplar, fir, plantain, etc), tea, coffea, oil palm, coconut, vegetable species, such as tomato (Lycopersicon ssp e.g. Lycopersicon esculentum), potato (Solanum tuberosum, other Solanum species), eggplant (Solanum melongena), peppers (Capsicum annum, Capsicum frutescens), pea, zucchini, beans (e.g. Phaseolus species), cucumber, artichoke, asparagus, broccoli, garlic, leek, lettuce, onion, radish, turnip, Brussels sprouts, carrot, cauliflower, chicory, celery, spinach, endive, fennel, beet, fleshy fruit bearing plants (grapes, peaches, plums, strawberry, mango, apple, plum, cherry, apricot, banana, blackberry, blueberry, citrus, kiwi, figs, lemon, lime, nectarines, raspberry, watermelon, orange, grapefruit, etc.), ornamental species (e.g. Rose, Petunia, Chrysanthemum, Lily, Gerbera species), herbs (mint, parsley, basil, thyme, etc.), woody trees (e.g. species of Populus, Salix, Quercus, Eucalyptus), fibre species e.g. flax (Linum usitatissimum) and hemp (Cannabis sativa).

The host species may already produce a functional PINH protein, in which cases the transformation with an additional PINH encoding gene may enhance PINH levels in the cell and may still modify the flavour and/or fragrance by hydroxylating more substrate. A skilled person will easily know how to determine whether a significant change in flavour and/or odour is detectable in the recombinant plant or plant tissue compared to the control plant or tissue. For example simple sensory tests, as commonly applied in the food industry, can be used, whereby taste, texture, fragrance, aftertaste, etc. are compared to appropriate controls. A significant change may for example be a more campherous, minty, woody odor and/or a more cooling, minty, campherous taste due to enhanced myrtenol levels or a more fresh, woody, minty odor and/or a more fresh, woody, herbaceous, carrot taste due to enhanced myrtenyl acetate levels. Comparison of the taste/odor to commercially available, chemically synthesized or extracted, myrtenol or myrtenyl acetate may be desirable. Obviously, it is desired to select plants which comprise a more pleasant flavour and/or fragrance or parts having a more pleasant fragrance and/or flavour.

The construction of chimeric genes and vectors for, preferably stable, introduction of PINH protein encoding nucleic acid sequences into the genome of host cells is generally known in the art. To generate a chimeric gene the nucleic acid sequence encoding a PINH protein according to the invention is operably linked to a promoter sequence, suitable for expression in the host cells, using standard molecular biology techniques. The promoter sequence may already be present in a vector so that the PINH nucleic sequence is simply inserted into the vector downstream of the promoter sequence. The vector is then used to transform the host cells and the chimeric gene is inserted in the nuclear genome or into the plastid, mitochondrial or chloroplast genome and expressed there using a suitable promoter (e.g., Mc Bride et al, 1995 Bio/Technology 13, 362; U.S. Pat. No. 5,693,507). In one embodiment a chimeric gene comprises a suitable promoter for expression in plant cells, operably linked thereto a nucleic acid sequence encoding a functional PINH protein according to the invention, optionally followed by a 3′nontranslated nucleic acid sequence.

The PINH nucleic acid sequence, preferably the PINH chimeric gene, encoding a functional PINH protein, can be stably inserted in a conventional manner into the nuclear genome of a single plant cell, and the so-transformed plant cell can be used in a conventional manner to produce a transformed plant that has an altered phenotype due to the presence of the PINH protein in certain cells at a certain time. In this regard, a T-DNA vector, comprising a nucleic acid sequence encoding a PINH protein, in Agrobacterium tumefaciens can be used to transform the plant cell, and thereafter, a transformed plant can be regenerated from the transformed plant cell using the procedures described, for example, in EP 0 116 718, EP 0 270 822, PCT publication WO84/02913 and published European Patent application EP 0 242 246 and in Gould et al. (1991, Plant Physiol. 95, 426-434). The construction of a T-DNA vector for Agrobacterium mediated plant transformation is well known in the art. The T-DNA vector may be either a binary vector as described in EP 0 120 561 and EP 0 120 515 or a co-integrate vector which can integrate into the Agrobacterium Ti-plasmid by homologous recombination, as described in EP 0 116 718.

Preferred T-DNA vectors each contain a promoter operably linked to PINH encoding nucleic acid sequence between T-DNA border sequences, or at least located to the left of the right border sequence. Border sequences are described in Gielen et al. (1984, EMBO J. 3, 835-845). Of course, other types of vectors can be used to transform the plant cell, using procedures such as direct gene transfer (as described, for example in EP 0 223 247), pollen mediated transformation (as described, for example in EP 0 270 356 and WO85/01856), protoplast transformation as, for example, described in U.S. Pat. No. 4,684,611, plant RNA virus-mediated transformation (as described, for example in EP 0 067 553 and U.S. Pat. No. 4,407,956), liposome-mediated transformation (as described, for example in U.S. Pat. No. 4,536,475), and other methods such as those described methods for transforming certain lines of corn (e.g., U.S. Pat. No. 6,140,553; Fromm et al., 1990, Bio/Technology 8, 833-839; Gordon-Kamm et al., 1990, The Plant Cell 2, 603-618) and rice (Shimamoto et al., 1989, Nature 338, 274-276; Datta et al. 1990, Bio/Technology 8, 736-740) and the method for transforming monocots generally (PCT publication WO92/09696). The most widely used transformation method for dicot species is Agrobacterium mediated transformation. For cotton transformation see also WO 00/1733. Brassica species (e.g. cabbage species, broccoli, cauliflower, rapeseed etc.) can for example be transformed as described in U.S. Pat. No. 5,750,871 and legume species as described in U.S. Pat. No. 5,565,346. Musa species (e.g. banana) may be transformed as described in U.S. Pat. No. 5,792,935. Agrobacterium-mediated transformation of strawberry is described in Plant Science, 69, 79-94 (1990). Likewise, selection and regeneration of transformed plants from transformed cells is well known in the art. Obviously, for different species and even for different varieties or cultivars of a single species, protocols are specifically adapted for regenerating transformants at high frequency.

Besides transformation of the nuclear genome, also transformation of the plastid genome, preferably chloroplast genome, is included in the invention. One advantage of plastid genome transformation is that the risk of spread of the transgene(s) can be reduced. Plastid genome transformation can be carried out as known in the art, see e.g. Sidorov V A et al. 1999, Plant J. 19: 209-216 or Lutz K A et al. 2004, Plant J. 37(6):906-13, U.S. Pat. No. 6,541,682, U.S. Pat. No. 6,515,206, U.S. Pat. No. 6,512,162 or U.S. Pat. No. 6,492,578.

The PINH nucleic acid sequence is inserted in a plant cell genome so that the inserted coding sequence is downstream (i.e. 3′) of, and under the control of, a promoter which can direct the expression in the plant cell. This is preferably accomplished by inserting the chimeric gene in the plant cell genome, particularly in the nuclear or plastid (e.g. chloroplast) genome.

Preferred promoters include: the strong constitutive 35S promoters or (double) enhanced 35S promoters (the “35S promoters”) of the cauliflower mosaic virus (CaMV) of isolates CM 1841 (Gardner et al., 1981, Nucleic Acids Research 9; 2871-2887), CabbB-S (Franck et al., 1980, Cell 21, 285-294) and CabbB-JI (Hull and Howell, 1987, Virology 86, 482-493); the 35S promoter described by Odell et al. (1985, Nature 313, 810-812) or in U.S. Pat. No. 5,164,316, promoters from the ubiquitin family (e.g. the maize ubiquitin promoter of Christensen et al., 1992, Plant Mol. Biol. 18, 675-689, EP 0 342 926, see also Cornejo et al. 1993, Plant Mol. Biol. 23, 567-581), the gos2 promoter (de Pater et al., 1992 Plant J. 2, 834-844), the emu promoter (Last et al., 1990, Theor. Appl. Genet. 81, 581-588), Arabidopsis actin promoters such as the promoter described by An et al. (1996, Plant J. 10, 107.), rice actin promoters such as the promoter described by Zhang et al. (1991, The Plant Cell 3, 1155-1165) and the promoter described in U.S. Pat. No. 5,641,876 or the rice actin 2 promoter as described in WO070067; promoters of the Cassava vein mosaic virus (WO 97/48819, Verdaguer et al. 1998, Plant Mol. Biol. 37, 1055-1067), the pPLEX series of promoters from Subterranean Clover Stunt Virus (WO 96/06932, particularly the S7 promoter), a alcohol dehydrogenase promoter, e.g., pAdh1S (GenBank accession numbers X04049, X00581), and the TR1′ promoter and the TR2′ promoter (the “TR1′promoter” and “TR2′promoter”, respectively) which drive the expression of the 1′ and 2′ genes, respectively, of the T-DNA (Velten et al., 1984, EMBO J. 3, 2723-2730), the Figwort Mosaic Virus promoter described in U.S. Pat. No. 6,051,753 and in EP426641, histone gene promoters, such as the Ph4a748 promoter from Arabidopsis (PMB 8: 179-191), or others.

Alternatively, a promoter can be utilized which is not constitutive but rather is specific for one or more tissues or organs of the plant (tissue preferred/tissue specific, including developmentally regulated promoters), for example fruit (or fruit development or ripening) preferred, leaf preferred, epidermis preferred, root preferred, flower tissue preferred, seed preferred, pod preferred, stem preferred, whereby the PINH gene is expressed only in cells of the specific tissue(s) or organ(s) and/or only during a certain developmental stage, for example during fruit ripening. For example, the PINH gene(s) can be selectively expressed in green tissue/aerial parts of a plant by placing the coding sequence under the control of a light-inducible promoter such as the promoter of the ribulose-1,5-bisphosphate carboxylase small subunit gene of the plant itself or of another plant, such as pea, as disclosed in U.S. Pat. No. 5,254,799 or Arabidopsis as disclosed in U.S. Pat. No. 5,034,322. The choice of the promoter is obviously determined by the phenotype one aims to achieve, as described above. For example, to achieve fruits (e.g. tomatoes, strawberries, etc.) with a modified taste and/or fragrance a fruit specific or fruit preferred promoter is the most suitable. A suitable promoter to confer expression to fruits is for example a fruit and peel specific promoter e.g. beta-Galactosidase II (Smith et al., 1998, Plant Physiol 117: 417-23). Other fruit development and/or ripening-specific promoters that could be used include the ripening-enhanced polygacturonase promoter (WO92/08798), the E4 or E8 promoter (U.S. Pat. No. 5,859,330; Diekman & Fischer, 1988, EMBO, 7:3315-3320), the fruit specific 2AII promoter (Pear et al, 1989, Plant Molecular Biology, 13:639-651), the ERT promoters described in U.S. Pat. No. 5,908,973 or others. A whole range of other fruit specific or fruit ripening associated promoters have been isolated and may be used. For example a banana fruit specific promoter may be used (e.g. WO0056863), strawberry fruit specific promoters that express selectively in receptacle tissue (see U.S. Pat. No. 6,043,410), etc. A receptacle tissue preferred promoter is particularly suitable for expression of PINH in plant species wherein a major portion of the edible fruit comprises receptacle tissue, such as strawberry, apple, pear, quince. Other promoters, e.g. fruit preferred promoters or root-specific, can be identified and isolated by one skilled in the art, using for example microarrays and confirmation of the in vivo expression profile by transformation of promoter reporter gene fusions.

To achieve flowers with modified fragrance a flower tissue specific promoter (e.g. a petal or sepal specific promoter, an ovary specific promoter, etc.) may be the most suitable. A petal specific promoter has been described in WO9915679.

To modify the flavour and/or fragrance of aerial plant parts a constitutive, a leaf specific, epidermis specific or light-inducible promoter would be suitable. Suitable epidermal specific promoters, such as for example the Arabidopsis LTP1 promoter (Thoma et al, 1994, Plant Physiol. 105(1):35-45.), the CER1 promoter (Aarts et al 1995. Plant Cell. 7:2115-27), and the CER6 promoter (Hooker et al 2002, Plant Physiol 129:1568-80.) and the orthologous tomato LeCER6 (Vogg et al, 2004, J. Exp Bot. 55: 1401-10), provide specific expression in above ground epidermal surfaces.

To modify the fragrance and/or flavour of seeds, a seed specific promoter, as described in EP723019, EP255378 or WO9845461 can be used. For tuber specific expression (e.g. potatoes) a tuber or peel specific promoter is the most suitable such as the class II patatin promoter (Nap et al, 1992, Plant Mol. Biol. 20: 683-94.) that specifies expression in the outer layer of the tuber, or a promoter with leaf and tuber peel expression such as the potato UBI7 promoter (Garbarino et al., 1995, Plant Physiol., 109: 1371-8). For root specific expression a promoter preferentially active in roots is described in WO00/29566. Another promoter for root preferential expression is the ZRP promoter (and modifications thereof) as described in U.S. Pat. No. 5,633,363.

Another alternative is to use a promoter whose expression is inducible. The modified flavour and/or fragrance may thus only develop after induction of PINH gene expression, for example upon a change in temperature, wounding, microbial or insect attack, chemical treatment (e.g. substrate-inducible) etc. Examples of inducible promoters are wound-inducible promoters, such as the MPI promoter described by Cordera et al. (1994, The Plant Journal 6, 141), which is induced by wounding (such as caused by insect or physical wounding), or the COMPTII promoter (WO0056897) or the promoter described in U.S. Pat. No. 6,031,151. Alternatively the promoter may be inducible by a chemical, such as dexamethasone as described by Aoyama and Chua (1997, Plant Journal 11: 605-612) and in U.S. Pat. No. 6,063,985 or by tetracycline (TOPFREE or TOP 10 promoter, see Gatz, 1997, Annu Rev Plant Physiol Plant Mol. Biol. 48: 89-108 and Love et al. 2000, Plant J. 21: 579-88). Other inducible promoters are for example inducible by a change in temperature, such as the heat shock promoter described in U.S. Pat. No. 5,447,858, by anaerobic conditions (e.g. the maize ADH1S promoter), by light (U.S. Pat. No. 6,455,760), by pathogens (e.g. EP759085 or EP309862) or by senescence (SAG12 and SAG13, see U.S. Pat. No. 5,689,042). Obviously, there are a range of other promoters available. A podwall specific promoter from Arabidopsis is the FUL promoter (also referred to as AGL8 promoter, WO9900502; WO9900503; Liljegren et al. 2004 Cell. 116(6):843-53)), the Arabidopsis IND1 promoter (Lijegren et al. 2004, supra.; WO9900502; WO9900503) or the dehiscence zone specific promoter of a Brassica polygalacturonase gene (WO9713856).

The PINH coding sequence is inserted into the plant genome so that the coding sequence is upstream (i.e. 5′) of suitable 3′ end transcription regulation signals (“3′end”) (i.e. transcript formation and polyadenylation signals). Polyadenylation and transcript formation signals include those of the CaMV 35S gene (“3′ 35S”), the nopaline synthase gene (“3′ nos”) (Depicker et al., 1982 J. Molec. Appl. Genetics 1, 561-573.), the octopine synthase gene (“3′ocs”) (Gielen et al., 1984, EMBO J 3, 835-845) and the T-DNA gene 7 (“3′ gene 7”) (Velten and Schell, 1985, Nucleic Acids Research 13, 6981-6998), which act as 3′-untranslated DNA sequences in transformed plant cells, and others.

Introduction of the T-DNA vector into Agrobacterium can be carried out using known methods, such as electroporation or triparental mating.

A PINH encoding nucleic acid sequence can optionally be inserted in the plant genome as a hybrid gene sequence whereby the PINH sequence is linked in-frame to a (U.S. Pat. No. 5,254,799; Vaeck et al., 1987, Nature 328, 33-37) gene encoding a selectable or scorable marker, such as for example the neo (or nptII) gene (EP 0 242 236) encoding kanamycin resistance, so that the plant expresses a fusion protein which is easily detectable.

Preferably, for selection purposes but also for weed control options, the transgenic plants of the invention are also transformed with a DNA encoding a protein conferring resistance to herbicide, such as a broad-spectrum herbicide, for example herbicides based on glufosinate ammonium as active ingredient (e.g. Liberty® or BASTA; resistance is conferred by the PAT or bar gene; see EP 0 242 236 and EP 0 242 246) or glyphosate (e.g. RoundUp®; resistance is conferred by EPSPS genes, see e.g. EPO 508 909 and EP 0 507 698). Using herbicide resistance genes (or other genes conferring a desired phenotype) as selectable marker further has the advantage that the introduction of antibiotic resistance genes can be avoided. Alternatively, other selectable marker genes may be used, such as antibiotic resistance genes. As it is generally not accepted to retain antibiotic resistance genes in the transformed host plants, these genes can be removed again following selection of the transformants. Different technologies exist for removal of transgenes. One method to achieve removal is by flanking the chimeric gene with lox sites and, following selection, crossing the transformed plant with a CRE recombinase-expressing plant (see e.g. EP506763B1). Site specific recombination results in excision of the marker gene. Another site specific recombination systems is the FLP/FRT system described in EP686191 and U.S. Pat. No. 5,527,695. Site specific recombination systems such as CRE/LOX and FLP/FRT may also be used for gene stacking purposes. Further, one-component excision systems have been described, see e.g. WO9737012 or WO9500555).

When reference to “a transgenic plant cell” or “a recombinant plant cell” is made anywhere herein, this refers to a plant cell (or also a plant protoplast) as such in isolation or in tissue/cell culture, or to a plant cell (or protoplast) contained in a plant or in a differentiated organ or tissue, and these possibilities are specifically included herein. Hence, a reference to a plant cell in the description or claims is not meant to refer only to isolated cells in culture, but refers to any plant cell, wherever it may be located or in whatever type of plant tissue or organ it may be present. Also, parts removed from the recombinant plant, such as harvested fruit, seeds, cut flowers, pollen, etc. as well as cells derived from the recombinant cells, such as seeds derived from traditional breeding (crossing, selfing, etc.) which retain the chimeric PINH gene are specifically included.

### 1.2 Silencing

In one embodiment the natural flavour and/or fragrance is modified by gene silencing of the endogenous PINH gene or gene family. Especially, silencing results in plants and/or tissues which produce significantly reduced amounts of the PINH-hydroxylation product (and/or further derivatives), such as myrtenol and/or myrtenol derivatives, e.g. myrtenyl acetate. For example, wild strawberry transformed with a PINH gene silencing construct under control of a constitutive or a fruit specific promoter are modified in flavour and or taste, due to an increase of alpha-pinene and a significant reduction (or complete absence) of myrtenol and/or myrtenyl acetate. A “significant reduction” refers herein to a reduction of at least 5%, 10%, 20%, 30%, 40%, 50%, 70%, 80%, 90%, 95% or 100% less PINH-hydroxylated (mono)terpene product, especially C10 and/or C7 hydroxylated (mono)terpene or (mono)terpene analogue products, than the control plant/cell/tissue. It is understood that endogenous expression of other enzymes involved in terpene biosynthesis or catabolism may additionally be silenced using an appropriate gene silencing construct, for example endogenous AAT may be silenced.

“Gene silencing” refers to the down-regulation or complete inhibition of gene expression of one or more target genes (e.g. endogenous PINH and/or AAT). The use of inhibitory RNA to reduce or abolish gene expression is well established in the art and is the subject of several reviews (e.g. Baulcombe 1996, Stam et al. 1997, Depicker and Van Montagu, 1997). There are a number of technologies available to achieve gene silencing in plants, such as chimeric genes which produce antisense RNA of all or part of the target gene (see e.g. EP 0140308 B1, EP 0240208 B1 and EP 0223399 B1), or which produce sense RNA (also referred to as co-suppression), see EP 0465572 B1.

The most successful approach so far has however been the production of both sense and antisense RNA of the target gene (“inverted repeats”), which forms double stranded RNA (dsRNA) in the cell and silences the target gene. Methods and vectors for dsRNA production and gene silencing have been described in EP 1068311, EP 983370 A1, EP 1042462 A1, EP 1071762 A1 and EP 1080208 A1. A vector according to the invention may therefore comprise a transcription regulatory region which is active in plant cells operably linked to a sense and/or antisense DNA fragment of a PINH gene according to the invention. Generally short (sense and antisense) stretches of the target gene sequence, such as 17, 18, 19, 20, 21, 22 or 23 nucleotides of cording or non-coding sequence are sufficient. Longer sequences can also be used, such as 100, 200 or 250 nucleotides. Preferably, the short sense and antisense fragments are separated by a spacer sequence, such as an intron, which forms a loop (or hairpin) upon dsRNA formation. Any short stretch of SEQ ID NO: 1-3 may be used to make a PINH gene silencing vector and a transgenic plant in which one or more PINH genes are silenced in all or some tissues or organs. A convenient way of generating hairpin constructs is to use generic vectors such as pHANNIBAL and pHELLSGATE, vectors based on the Gateway® technology (see Wesley et al. 2004, Methods Mol. Biol. 265:117-30; Wesley et al. 2003, Methods Mol. Biol. 236:273-86 and Helliwell & Waterhouse 2003, Methods 30(4):289-95.), all incorporated herein by reference.

By choosing conserved nucleic acid parts of the PINH gene, PINH family members in a host plant can be silenced. Encompassed herein are also transgenic plants comprising a transcription regulatory element operably linked to a sense and/or antisense DNA fragment of a PINH gene and exhibiting a PINH gene silencing phenotype. Gene silencing constructs may also be used in reverse genetic approaches, to elucidate or confirm the function of a PINH gene or gene family in a host species.

### 1.3 Uses of the Recombinant Plant Host Cells

The resulting transformed plant can be used in a conventional plant breeding scheme to produce more transformed plants with the same characteristics or to introduce the transgene into other varieties of the same or related plant species. Seeds, which are obtained from the transformed plants, preferably contain the chimeric PINH gene (or silencing construct) as a stable genomic insert, and optionally one or more other chimeric genes as described above. Cells of the transformed plant or the whole plant can be cultured in a conventional manner to recover the (mono)terpene products. The modified cells, plant tissue or organs may be used as such or may be processed further.

In one embodiment a plant, plant cells or plant tissue is provided (e.g. a fruit, seeds, flower, etc.) which has a significantly modified flavour and/or fragrance (optionally only following induction or external substrate supply). The tissue may be used directly, for example eaten without any processing, as decoration or may be used to make food or feed products having a more pleasant fragrance and/or flavour (e.g. fruit cakes, fruit or vegetable juices, other beverages, soup, deserts, etc.). “Food” refers herein to products consumed by humans, including not only solid and semi-solid food but also beverages. Food also encompasses gaseous products which are inhaled, such as cigarettes, cigars, inhalants, etc. “Feed” refers herein to products consumed by non-human animals, especially vertebrates, such as farm animals or pets. Thus, food and feed compositions comprising a modified flavour and/or fragrance (compared to the analogous product made using control plant parts) are part of the invention. These products can be distinguished from other products not only by the flavour and/or fragrance, but also by the presence of all or part of the recombinant PINH DNA or protein, using for example PCR or immunological detection techniques.

In another embodiment, the plant cells are used to make fractions with enhanced amounts of PINH-hydroxylation products (and/or further derivatives thereof), such as C10 (or C9) and/or C7 hydroxylated monoterpenes (see FIGS. 4 and 5; at least any of the products shown, or further derivatives thereof, may be produced), e.g. myrtenol and/or myrtenol derivatives, or perilla alcohol and/or limonene-10-ol or further derivatives thereof. Optionally the PINH-hydroxylation products may be substantially purified from the tissue using known methods. Either the fractions or the substantially purified products may be used to manufacture various compositions, such as fragrances or flavouring agents or compositions comprising modified fragrance and/or flavour characteristics. For example cosmetic compositions, such as creams, perfumes, make-up, lotions, deodorants, shower gels, etc. may be made using a suitable amount of recombinant plant extract or purified PINH-hydroxylation products obtainable by extraction/purification from the plant cells. Other compositions, such as detergents, plastics, soaps, glues, etc. with modified scent may also be made. Equally, flavourings or flavour additives suitable for human or animal consumption are provided, or product comprising these, which contain a PINH-hydroxylation product obtainable from a recombinant plant cells as described. Thus, a composition comprising a suitable amount of PINH-hydroxylated monoterpene or monoterpene analogue product, having a carbon skeleton essentially similar to that of α-pinene or limonene (see FIGS. 4 and 5), and being obtainable from a recombinant plant cell, tissue or plant comprising a PINH encoding nucleic acid sequence under the control of a suitable transcription regulatory sequence active in plant cells is provided. Preferred products are perilla alcohol, myrtenol, limonene-10-ol and other analogously hydroxylated terpenoids and aromatic compounds. After hydroxylation these products can easily be further modified, e.g. by chemical oxidation or the use of enzymes (alcohol dehydrogenases) to produce aldehydes. Aldehydes, such as myrtenal or limonene-10-al have interesting properties for the flavour industry or for crop protection purposes (bio-control). The alcohols produced by PINH may also be used as substrates for esterification.

2. Recombinant Host Cells with Improved Resistance or Comprising Bio-Control Activity

As terpenes and terpene analogous play a role in plant defence, they may be used as bio-control agents. In one embodiment plants with modified, especially with significantly enhanced levels of PINH-hydroxylation products and/or derivatives thereof, such as myrtenol and/or derivatives (e.g. myrtenal), perillyl alcohol and/or derivatives (e.g. perilla aldehyde and/or perillic acid), etc. (see elsewhere herein) are provided, whereby these plants have significantly enhanced levels of resistance to plant pests and/or pathogens. In another embodiment the attractiveness to insects of plants is decreased by gene silencing of the endogenous PINH gene or gene family. Especially, silencing results in plants and/or tissues which produce significantly reduced amounts of the PINH-hydroxylation product (and/or derivatives), such as myrtenol and/or myrtenol derivatives, so that they are less attractive to pest insects. PINH overexpressing or silenced plants can be generated as described above, whereby selection of the recombinant plants is based on bioassays and/or suitable analytical techniques such as GC-MS and LC-MS. Suitable promoters used for PINH expression are constitutive promoters, wound-inducible promoters, pest or pathogen inducible promoters, tissue specific promoters and the like. As described, a skilled person can easily determine whether to introduce PINH alone or additionally one or more PINH-substrate producing enzyme and/or product modifying enzymes into the host cell. Methods for making transformation vectors and recombinant plants are described elsewhere herein.

Pest and pathogen resistance levels of recombinant plants compared to controls can be tested in green-house bioassays or preferably in field trials with a high disease pressure and a diverse pest/pathogen population. “Resistance” refers herein to both a significant reduction in damage caused when exposed to the pest or pathogen, as well as a complete absence of damage. A “significant reduction in damage” (or “significantly enhanced resistance”) refers to a reduction by at least 3%, 5%, 10%, 15%, 20% or more compared to control plants. This may be measured directly by scoring damage symptoms (e.g. leaf lesions, etc.) or indirectly, for example by determining yield. Assays for scoring pest and/or pathogen damage are known in the art. Obviously, for different pests/pathogens bioassays differ, depending on the life-cycle of the pest/pathogen and the symptoms of the host plant or tissue. In general terms, a bioassay generally involves growing recombinant plants and control plants under conditions whereby they come into contact with the pest/pathogen (e.g. following inoculations) and scoring the resistance at one or more time-points and on one or more tissues. Statistical analysis is then used to determine whether significant differences in resistance levels between the recombinant plants and the control plants exist. As the position of integration in the genome may influence the phenotype, normally a number of transformation events are compared in order to select a so-called “elite event” with high resistance. Further, elite event detection kits (such as PCR detection kits) based for example on the integrated sequence and the flanking (genomic) sequence are developed using known methods (see e.g. WO0141558).

It was especially found that PINH genes according to the invention can be used to provide protection levels higher than provided by recombinant plants producing linalool (see WO02/064764).

The underlying mechanisms is not relevant, but may be a reduction of mycelial growth and/or spore germination, etc. caused by the PINH product or further derivative thereof (see Examples). “Broad spectrum resistance” refers to resistance to a range of pathogens and/or pests. Resistant plants have the advantage that less or no chemicals need to be used to protect the crop plants. Plants according to the invention comprise at least a nucleic acid sequence encoding a functional PINH enzyme according to the invention and significantly enhanced resistance to one or more pest and/or pathogen species compared to control plants. Especially, recombinant plants with significantly enhanced resistance against fungal pathogens (Fusarium species, Leptosphaeria, Sclerotinia, Botrytis spp., Pythium spp., etc.), oomycetes (e.g. Phytophthora infestans), bacterial pathogens (Bacillus thuringiensis, Pseudomonas species, etc.), insect pests (coleoptera, lepidoptera, hemiptera, etc.), nematodes and herbivore pests (e.g. rabbits, pigeons, deer) are provided.

Pathogens of the invention therefore include, but are not limited to, viruses or viroids, bacteria, insects, nematodes, fungi, parasitic plants, and the like. Viruses include any plant virus, for example, tobacco mosaic virus, cucumber mosaic virus, ringspot virus, necrosis virus, maize dwarf mosaic virus, etc. Each crop plant is host to specific pests and pathogens. Recombinant soybean plants with significantly enhanced resistance to one or more of the following pathogens are provided: Phytophthora megasperma fsp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kiluchii, Cercospora sojina, Peronospora manshurica, Colletotrichum dematium (Colletotichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidernatum, Pythium ultimum, Pythium debaryanum, Tomato spotted wilt virus, Heterodera glycines Fusarium solani.

Recombinant oilseed rape (or Canola) plants with significantly enhanced resistance to one or more of the following pathogens are provided: Albugo candida, Alternaria brassicae, Leptosphaeria maculans, Pyrenopeziza brassicae, Rhizoctonia solani, Sclerotinia sclerotiorum, Mycosphaerella brassiccola, Pythium ultimum, Peronospora parasitica, Fusarium roseum, Alternaria alternate.

Recombinant alfalfa plants with significantly enhanced resistance to one or more of the following pathogens are provided: Clavibater michiganese subsp. insidiosum, Pythium ultimum, Pythium irregulare, Pythium splendens, Pythium debaryanum, Pythium aphanidermatum, Phytophthora megaspenna, Peronospora trifoliorum, Phoma medicaginis var. medicaginis, Cercospora medicaginis, Pseudopeziza medicaginis, Leptotrochila medicaginis, Fusarium, Xanthomonas campestris p.v. alfalfae, Aphanomyces euteiches, Stemphylium herbarum, Stemphylium alfalfae.

Recombinant wheat plants with significantly enhanced resistance to one or more of the following pathogens are provided: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis f.sp. tritici, Puccinia recondita f.sp. tritici, Puccinia striiformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidennatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Ustilago tritici, Tilletia indica, Rhizoctonia solani, Pythium arrhenomannes, Pythium gramicola, Pythium aphanidemmatum, High Plains Virus, European wheat striate virus.

Recombinant sunflower plants with significantly enhanced resistance to one or more of the following pathogens are provided: Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum pv. carotovora, Cephalosporium acremonium, Phytophthora cryptogea, Albugo tragopogonis.

Recombinant maize plants with significantly enhanced resistance to one or more of the following pathogens are provided: Fusarium moniliforme var. subglutinans, Erwinia stewartii, Fusarium moniliforme, Gibberella zeae (Fusarium graminearum), Stenocarpella maydi (Diplodia maydis), Pythium irregulare, Pythium debaryanum, Pythium graminicola, Pythium splendens, Pythium ultimum, Pythium aphanidennatum, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella-maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi pv. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronoscierospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus.

Recombinant sorghum plants with significantly enhanced resistance to one or more of the following pathogens are provided: Exserohilum turcicum, Colletotrichum graminicola (Glomerella graminicola), Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Perconia circinata, Fusarium moniliforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola, etc. Resistance to nematodes includes parasitic nematodes such as root knot, cyst, lesion, and reniform nematodes, etc. Resistance to parasitic plants includes mistletoes, witchweeds (Striga spp.), Orobanche cumana and other parasitic Orobanche spp.

Plants resistant to insects include resistance to insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera.

Thus, maize plants with significantly enhanced resistance to one or more of the following insects is provided: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, sugarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite.

Wheat plants with resistance to one or more of the following insects are provided: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite.

Further, sunflower plants with resistance to one or more of the following insects are provided: Suleima helianthana, sunflower bud moth; Homoeosoma electellum, sunflower moth; zygogramma exclaniationis, sunflower beetle; Bothyrus gibbosus, carrot beetle; Neolasioptera murtfeldtiana, sunflower seed midge. Cotton plants resistant to one or more of the following insects are provided: Heliothis virescens, cotton budworm; Helicoverpa zea, cotton bollworm; Spodoptera exigua, beet armyworm; Pectinophora gossypiella, pink bollworm; Anthonomus grandis grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinliella fusca, tobacco thrips; Tetranychus cinnabarinus, carmine spider mite; Tetranychus urticae, twospotted spider mite. Rice plants with resistance to one or more of the following insects are provided: Diatraea saccharalis, sugarcane borer; Spodoptera frugiperda, fall armyworm; Helicoveipa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite. Barley with resistance to the following insects is provided: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite. Oilseed rape (or canola) plants with resistance to one or more of the following insects are provided: Brevicoryne brassicae, cabbage aphid; Phyllotreta cruciferae, Flea beetle; Mamestra configurata, Bertha armyworm; Plutella xylostella, Diamond-back moth; Delia ssp., Root maggots. The PINH gene according to the invention may also be used to make resistant plants and tissues of any of the other crop plants listed elsewhere herein.

In one embodiment plants or tissues with significantly enhanced resistance (or repellance) to aphids, especially Aphis fabae (black bean aphid), are provided. Such plants comprise significantly enhanced amounts of myrtenal, whereby the enhanced myrtenal levels are a result of the catalytic activity of a recombinant PINH enzyme in the plant tissue (and optionally one or more additional endogenous or recombinant enzymes). Preferred recombinant plants with enhanced resistance to Aphis fabae include field beans, broad beans and sugar beet, as well as most forms of garden bean.

In another embodiment plants with enhanced resistance to Phytophthora spp (e.g. P. infestans and/or Botrytis species (e.g. Botrytis elliptica) and/or Pythium species (e.g. P. aphiadermata) are provided (see Example 4 and FIG. 8). Especially, myrtenol and/or perilla alcohol and/or perilla aldehyde can be produced to provide significantly enhanced resistance against Botrytis and/or Phytophthora and/or Pythium species. Preferably, at least 5, 10, 20, 30, 40, 50% or more (e.g. 100%) of the perilla alcohol and/or myrtenol produced by the PINH enzyme is further modified by endogenous plant enzymes or recombinant enzymes into derivatives, especially acids, aldehydes and/or glycosylation products, which are even more effective biocontrol agents. FIGS. 7 and 8 show that, for example, perilla aldehyde is about 2, 3, 4-5 or 10 fold, or more, more effective as a biocontrol agent than linalool with respect to certain pests/pathogens. Thus, recombinant plants and tissues comprising pest/pathogen-inhibiting amounts (i.e. amounts causing a significant reduction in pest/pathogen damage) of perilla alcohol and/or myrtenol and/or myrtenal and/or perillic acid and/or perilla aldehyde or other derivatives thereof are provided. Preferred host plants include Solanaceae (especially potato), legumes, cereals, etc.

The plants (or parts thereof) also have an improved storage capability and a longer shelf life and are of better quality as for example toxins produced by pathogens are reduced. Many pests and pathogens, such as for example Fusarium species, cause significant post-harvest losses, by damaging the plant tissue during storage. The modified terpene and terpene-analogue levels and profiles result in a reduction of yield loss caused post-harvest by reducing the incidence and/or severity of damage.

Preferred host plants are crop plants, i.e. plants cultivated by humans for food, feed or ornamental purposes.

Also, the recombinant plant tissue may be ploughed into the soil prior to planting a new crop, which reduces the pathogen incidence in the emerging crop. Similar practices exist for Brassica species with high glucosinolate contents in the leaves.

In another embodiment recombinant microorganisms, such as bacteria (for example Pseudomonas, Agrobacterium, Bacillus or Escherichia), fingi (yeast species, e.g. Saccharomyces ssp, Hansenula, Pichia, Kluyveromyces, Candida, Aspergillus, Chrysosporium, etc.), viruses or algae expressing a functional PINH enzyme and producing a PINH-hydroxylation product and/or derivatives thereof (e.g. myrtenol, myrtenal, perilla alcohol, perilla aldehyde, perillic acid, benzylalcohol) from a suitable substrate (produced by the cell or provided externally) are provided. If the chosen host cells do not make the PINH substrate themselves, they may also be modified to produce the substrate, by introducing additional genes, or by selecting an already modified host cell, such as E. coli cells described by Martin et al. 2003 (Nature Biotechnology 21: 796-802). Also, if the host cell does not produce an endogenous electron donating protein (e.g. prokaryotic cells), it is necessary to also introduce a gene encoding such a protein into the cell or to chose a cell which already has a suitable electron donating protein integrated, such as the Saccharomyces cerevisiae strains described by Pompon et al 1996, supra, and by Urban et al. 1997, supra. Additionally, the N-terminal sequence of the PINH enzyme may need to be modified for correct PINH anchorage and folding, as described by Halkier et al. 1995 (supra) and elsewhere herein. Likewise PINH-hydroxylation product modifying enzymes may be introduced if the host cells does not have the required genes endogenously.

Such (live, viable or lysed) microorganisms can be used as active ingredients of biocontrol compositions, and may for example be sprayed onto the crop plants to protect these from pest or pathogen damage. A bio-control composition comprising such recombinant microorganism (and optionally a suitable PINH substrate if this is not produced in vivo by the host cell, e.g. α-pinene) is provided herein. Preferably, such a composition is in liquid or semi-solid form (foam, gel, etc,), so that it can be easily sprayed onto the aerial plant surface. For control of root or tuber pathogens or pests it may also be incorporated into the soil, and may alternatively be in granule, powder or solid form. See e.g. WO96/10083 for compositions. The composition comprising a recombinant microorganism may also be used as a seed coating. The PINH substrate may be in the composition medium, optionally together with additional components, such as nutrients, osmotic agents, suitable carriers, diluents, emulsifiers and/or dispersants (e.g. as described for Bacillus thuringiensis by Bernhard and Utz, 1993, in Bacillus thuringiensis, An Environmental Biopesticide: Theory and Practice, pp. 255-267). Etc. The recombinant microorganism may also be grown in large scale cultures and the cells may be lysed prior to or during preparation of the biocontrol composition, releasing the PINH hydroxylation products and/or derivatives thereof (e.g. myrtenol). Alternatively, the PINH hydroxylation products and/or derivatives thereof may be extracted or purified from the microorganism and used as a bioactive component in the composition.

Transformation of bacteria with a PINH encoding gene of this invention can be carried out in a conventional manner, e.g. using conventional electroporation techniques as described in Maillon et al (1989 FEMS Microbiol. Letters 60, 205-210.) and in PCT Patent publication WO 90/06999. Also other microorganisms can be transformed using known methods, as described further below.

For expression in prokaryotic host cell, the codon usage of the nucleic acid sequence may be optimized accordingly (as described above). Intron sequences should be removed and other adaptations for optimal expression may be made as known (e.g. N-terminal modifications of the PINH enzyme).

3. Recombinant Host Cells with Insect Attractant Activity

It has already been mentioned that terpenes and terpene analogous act as attractants to certain insect species. It is, therefore, an embodiment of the invention to provide recombinant host cells, especially plants or plant parts or extracts, with modified terpene levels and profiles, as described above, for use as insect attractants. Such plants can for example be planted in areas where the insects occur or as borders around crops in order to attract the insects to the recombinant plants. Alternatively parts of the plants can be used to prepare insect attracting compositions, e.g. to lure, capture or exterminate the insects so that they cannot cause damage to other plants. Traps comprising such insect-attracting plant material or compositions are part of the invention. Optionally, an insecticide is added to the traps or compositions, whereby the attracted insects are killed.

For example the pine shoot beetle, Tomicus piniperda (L.) (Coleoptera, Scolytidae), is a destructive insect pests affecting pines (Pinus sylvestris) in its native range of Europe and Asia. It has been found that α-pinene and myrtenol act synergistically, and that the combination attracted twice as many beetles as α-pinene alone (U.S. Pat. No. 6,203,786). It is an embodiment of the invention to provide recombinant plants and/or plant parts comprising (a) an increased amount of myrtenol or preferably (b) an increased amount of both myrtenol and α-pinene, preferably in a synergistically effective ratio. Especially, the pine shoot beetle attraction of the tissue or whole plant is increased by at least 5%, 10%, 20%, 30% or more compared to the attraction of α-pinene alone.

### 4. Bioconversion Systems Using PINH Nucleic Acid Sequences and Proteins

In yet another embodiment of the invention a PINH enzyme according to the invention is used in a bioconversion system in order to produce large amounts of C10 (or C9) and/or C7 hydroxylated monoterpenes or aromatic compounds (see FIGS. 4 and 5) and/or further derivatives thereof. A particularly preferred embodiment is the conversion of limonene to perillyl alcohol, but the conversion of other PINH substrates (as described elsewhere herein) is equally envisaged. One advantage of the use of PINH enzyme is that the hydroxylation product is substantially free of other unwanted hydroxylation products. Although also plants, animals, plant cell cultures and animal cell cultures may be used in this system, the use of microorganisms is preferred herein, as thereby large scale production of essentially pure PINH hydroxylation products, especially (+) or (−) perillyl alcohol is easier and cheaper.

Perillyl alcohol has been shown to have both chemoprotective and chemotherapeutic properties. It is currently undergoing both phase I and phase II clinical trials as a drug for the treatment or prevention of various cancers, such as breast- (mammary), ovarian, colorectal- and prostate cancer (Ripple et al. 2000, Clin Cancer Res. 6(2):390-6; Morgan-Meadows et al. 2003, Cancer Chemother Pharmacol 52(5):361-6; Liu et al. 2003, Invest New Drugs 21(3):367-72; see also the review by Belanger, 1998, Altern. Med. Rev. 3(6):448-57). Also the effect on other cancers, such as liver cancer, skin and lung cancer is being investigated. Perillyl alcohol is more effective than limonene, which also has some anticancer activity.

Although the precise mode of action of perillyl alcohol is unclear, drug-related activities that have been observed include the induction of apoptosis, cell cycle arrest, the inhibition of posttranslational modification of proteins that are involved in signal transduction, and differential gene regulation.

The method comprises the following steps:
a) introducing one or more nucleic acid sequences encoding a functional PINH enzyme according to the invention into a host cell,
b) growing the host cell under conditions whereby functional PINH enzyme is produced
c) supplying a suitable amount of PINH substrate to the growth medium or use host cells that are able to produce the substrate in vivo e.g. due to endogenous genes or due to expression of one or more recombinant genes
d) alternatively to step c) the intact cells may be exposed to externally applied substrate or first lysed and/or cell fractions (e.g. microsomal fractions) prepared and then brought in contact with the PINH substrate
e) isolating the PINH hydroxylation product (and/or one or more further derivatives thereof) from the cells or culture medium and optionally further purifying the PINH hydroxylation product (and/or the derivative(s)).

The method steps vary slightly, depending on the host cells used and the product that is to be produced. In principle any terpene or aromatic substrate recognized by the PINH enzyme (as described) may be converted using these methods.

The host system used for heterologous expression determines the type of modifications that need to be made to achieve functional PINH production. In other words, the host cell determines whether or not an additional electron-transfer protein encoding gene needs to be introduced, whether or not the nucleic acid sequence encoding the N-terminal part of the PINH enzyme needs to be modified for functional PINH expression, whether codon usage of the PINH coding sequence should be adapted and/or introns removed. As already described above, prokaryotic host cells such as E. coli need to be (co-) transformed with a heterologous electron donating protein, such as a yeast or plant NADPH-cytochrome P450 reductase. Another solution is to transform the host cell with a nucleic acid sequence encoding a (translational) fusion protein of the cytochrome P450:NADPH P450 reductase, as e.g. described by Hotze et al. 1995, FEBS Lett. 374:345-350.

Alternatively, a host strain can be chosen which has already been modified. Similarly eukaryotic host cells, such as yeast or fungal host cells, which already comprise an endogenous or recombinant electron donating protein may be chosen or the cells may be co-transformed with a nucleic acid sequence encoding a functional electron donating protein, especially a plant NADPH-cytochrome P450 reductase gene. Such genes are widely available, e.g. from Arabidopsis or Sorghum (see e.g. Urban et al. 1997, supra and Bak et al 1997, supra), from yeast (e.g. Y. lipolytica, see Nthangeni et al. Yeast 21: 583-592), fungi, etc. Generally, even if an endogenous electron donating protein is present in the host cell, co-expression of a homologous or heterologous CPR gene has been found to increase cytochrome P450 activity several fold (about 5 to 10-fold or more). Also, multi-copy transformants, comprising several PINH encoding genes, in addition to a (recombinant) CPR gene may further enhance PINH activity up to about 50-fold or more. Thus, both the introduction of one or more genes encoding CPR and/or PINH multicopy transformants are provided herein.

In one embodiment the host cell used for bioconversion is a microorganism, such as a gram-positive or gram-negative bacterial host cell (e.g. of the genus Escherichia, Rhodococcus, Bacillus, Mycobacterium, Cornybacterium, Arthrobacter, Staphylococcus, etc.) or a fungal host, such as a yeast cell selected from the genera Pichia (e.g. Pichia pastoris), Hansenula (e.g. Hansenula polymorpha), Saccharomyces (e.g. S. cerevisiae), Kluyveromyces (e.g. K. lactis), Yarrowia (e.g. Y. lipolytica), Arxula species (e.g. Arxula adeninivorans), Candida species and Schizosaccharomyces (e.g. S. pombe). In a preferred embodiment the host cell is a methylotrophic yeast, such as Pichia. In another embodiment the host cell is a filamentous fungal host selected from the genera Aspergillus, Trichoderma, Fusarium, Penicillium, Neurospora, Chrysosporium, Sporotrichum, Humicola, Sardoria and Acremonium.

Suitable expression vectors comprising PINH coding sequence can be either generated using known methods or can be obtained commercially. The transcription regulatory sequence is preferably strongly active in the host cell, either constitutively or following induction. A variety of transcription regulatory sequences capable of directing transcription in microbial host cells are available to the skilled person (Goosen et al., 1992, In: Handbook of Applied Mycology” 4: “Fungal Biotechnology”, and Romanos et al., 1992, Yeast 8: 423). Preferably the promoter sequence is derived from a highly expressed gene. Examples of preferred highly expressed genes from which promoters are preferably derived include but are not limited to genes encoding glycolytic enzymes such as triose-phosphate isomerases (TPI), glyceraldehyde-phosphate dehydrogenases (GAPDH), phosphoglycerate kinases (PGK), pyruvate kinases (PYK), alcohol dehydrogenases (ADH), as well as genes encoding amylases, glucoamylases, xylanases, cellobiohydrolases, beta-galactosidases, alcohol (methanol) oxidases, elongation factors and ribosomal proteins. Specific examples of suitable highly expressed genes include e.g. the LAC4 gene from Kluyveromyces sp., the methanol oxidase genes (AOX and MOX) from Hansenula and Pichia, respectively, the glucoamylase (glaA) genes from A. niger and A. awamori, the A. oryzae TAKA-amylase gene, the A. nidulans gpdA gene and the T. reesei cellobiohydrolase genes.

For expression in yeast species, such as Pichia or Hansenula species, for example the strong (methanol inducible) AOX1 promoter of the alcohol oxidase gene of Pichia (see U.S. Pat. No. 4,855,231), the Pichia pastoris alcohol oxidase II (AOX2 promoter) (Ohi et al., Mol. Gen. Genet. 243: 489-499, 1994) or the MOX1 promoter of Hansenula are suitable. Alternative promoters are the Pichia formaldehyde dehydrogenase promoter (FLD) as described in U.S. Pat. No. 6,730,499 and by Shen et al. Gene 216: 93-102, 1998, other yeast promoters, such as the 3-phosphoglycerate kinase promoter (PGK), glyceraldehyde-3-phosphate dehydrogenase (GAFDH or GAP) promoter, galactokinase (GAL1, GAL10) promoter, galactoepimerase promoter, and alcohol dehydrogenase (ADH1, ADRIII) promoter, the Pichia pastoris YPT1 promoter (Sears et al, Yeast 14: 783-790, 1998). Similarly, the strong POX promoters (e.g. POX2) of Y. lipolytica may be used (Pignede et al. 2000, Applied and Environmental Microbiology 66: 3283-3289).

A Pichia pastoris expression system is, for example, commercially available as a kit from Invitrogen, which uses the promoter and terminator from the AOX1 gene. Other, analogous expression systems may be used. Various expression vectors are available, such as integrative and autonomously replicating vectors (comprising an autonomous replicating sequence or ARS, as for example described in U.S. Pat. No. 4,837,148).

The expression vector preferably also comprises a selectable marker gene. The selectable marker may be any gene which confers a selectable phenotype upon the host and allows transformed cells to be identified and selected from untransformed cells. Suitable selectable markers which can be used for selection of the transformed host cells are well known to the skilled person (Goosen et al., 1992, In: Handbook of Applied Mycology” 4: “Fungal Biotechnology”, and Romanos et al., 1992, Yeast 8: 423). Preferred markers include but are not limited to e.g. versatile marker genes that can be used for transformation of most filamentous fingi and yeasts such as acetamidase genes or cDNAs (the amdS genes or cDNAs from A. nidulans, A. oryzae, or A. niger), or genes providing resistance to antibiotics like G418 or hygromycin or phleomycin. Alternatively, more specific selection markers can be used such as auxotrophic markers which require corresponding mutant host strains: e.g. URA3 (from S. cerevisiae or analogous genes from other yeasts), pyrG (from A. nidulans or A. niger) or argB (from A. nidulans or A. niger).

The selectable marker system may include an auxotrophic mutant methylotrophic yeast strain and a wild type gene which complements the host's defect. Examples of such systems include the Saccharomyces cerevisiae or Pichia pastoris HIS4 gene which may be used to complement his4 Pichia strains, or the S. cerevisiae or Pichia pastoris ARG4 gene which may be used to complement Pichia pastoris arg mutants, or the Pichia pastoris URA3 and ADE1 genes, which may be used to complement Pichia pastoris ura3 resp. ade1 mutants. Other selectable marker genes which function in Pichia pastoris include the zeo resistance gene, the G418 resistance gene, blasticidin resistance gene, and the like. Integration of the chimeric gene into the genome can be achieved by insertion or a transplacement into the region of the chromosomal AOX1 locus or integration may be targeted to the HIS4 locus.

Suitable promoters for mammalian cells include for example viral promoters, such as those from Simian Virus 40 (SV40), Rous sarcoma virus (RSV), adenovirus (ADV), and bovine papilloma virus. Suitable promoters for expression in transgenic plants or plant cell cultures are described above.

At the 3′ end of the coding sequence a 3′nontranslated nucleic acid sequence (3′ end) may be added, which may contain one or more transcription termination sites recognized by the host's transcription machinery. The origin of the 3′ end is not very critical and various suitable 3′ end sequences may be used. For example, the 3′ end sequence may be the 3′ end of the Pichia AOX1 gene, the Pichia HIS4 gene or the Pichia FLD1 gene. Preferably, for expression in yeast, a 3′ end of a yeast gene is used, for example of a gene naturally found in the host cell.

In another embodiment of the invention a microorganism, which comprises a nucleic acid sequence which encodes a functional PINH protein according to the invention, under control of a suitable promoter is provided. Especially a methylotropic yeast, preferably Pichia (e.g. Pichia pastoris or another readily transformable Pichia species) or Hansenula is provided, which, under suitable growth conditions produces high levels of functional PINH protein according to the invention. The microorganisms can be made by transforming a host strain with a vector as described above and selecting transformed cells. Preferred Pichia pastoris host strains are strains GS115 (NRRL Y-15851), GS190 (NRRL Y-18014), PPF1 (NRRL Y-18017), PPY120H, YGC4, and strains derived therefrom. Protease recognition sites, which may be recognized by host cell proteases, may be removed from the PINH sequence used known methods.

The vectors comprising the PINH encoding nucleic acid sequence under control of a suitable promoter can be introduced into the host cell using known methods. The chimeric gene may be integrated into the host cell genome or may remain in the cytoplasm, as a freely replicating unit. It is understood that the vector backbone preferably also comprises other elements required, such as an origin of replication, a selectable marker gene, etc. Transformation methods for yeast hosts include, for example, the spheroplast technique, described by Cregg et al. 1985, or the whole-cell lithium chloride yeast transformation system, Ito et al. (Agric. Biol. Chem. 48:341), or modified for use in Pichia as described in EP 312,934. Other published methods useful for transformation of the plasmids or linear vectors include U.S. Pat. No. 4,929,555; Hinnen et al. Proc. Nat. Acad. Sci. USA 75:1929 (1978); Ito et al. J. Bacteriol. 153:163 (1983); U.S. Pat. No. 4,879,231; Sreekrishna et al. Gene 59:115 (1987). Electroporation and PEG1000 whole cell transformation procedures may also be used, as described by Cregg and Russel, Methods in Molecular Biology: Pichia Protocols, Humana Press, Totowa, N.J., pp. 27-39 (1998). For filamentous fungi suitable transformation protocols are described in Goosen et al., 1992, In: Handbook of Applied Mycology” 4: “Fungal Biotechnology”, and in EP-A-0 635 574 and include for example protoplast transformation. Transformed host cells can be selected by using appropriate techniques including such as culturing auxotrophic cells after transformation in the absence of the biochemical product required (due to the cell's auxotrophy), selection for and detection of a new phenotype, or culturing in the presence of an antibiotic only allows growth of transformants comprising a resistance gene. Transformants can also be selected and/or verified by integration of the expression cassette into the genome, which can be assessed by, e.g., Southern Blot analysis or PCR. Additionally PINH enzyme activity assays can be carried out.

The recombinant host microorganisms is preferably grown under conditions leading to high expression of the PINH coding sequence according to the invention and the production of high amounts of PINH-hydroxylation product, following appropriate contact with a suitable substrate. For example expression levels of 2-3 g/l, 5-10 g/l or more are possible in yeasts. The substrate may be a single enantiomer or a mixture of enantiomers. It may be any substrate which is readily hydroxylated regiospecifically by the PINH enzyme according to the invention, as described elsewhere herein. For example alpha-pinene and limonene may be supplied to the medium or, in one embodiment synthesized directly by the host cell, e.g. from at least one other chimeric gene (encoding for example a limonene synthase and optionally also a GPP synthase) and with or without further optimisation of the host cell for monoterpene production, such as modifications leading to high-levels of isoprenoid precursor production (as described by Martin et al. 2003, supra). The substrate (e.g. (+) and/or (−)-limonene) may be added directly to the growth medium. It may be converted to the hydroxylation product (e.g. (+) and/or (−) perillyl alcohol) either in the medium (if the cells are lysed and/or membrane fractions are isolated at a certain stage in the process) or in vivo within the host cell (if the substrate or substrate precursor is taken up by the host cell). The location of the hydroxylation product may thus be either within the host cell or within the medium. When a single enantiomer is supplied, the bioconverted product typically consist of the single corresponding enantiomer.

The culturing conditions depend on the host strain and promoter used. Factors such as pH, temperature, nutrients, oxygen, co-factors etc. can be optimized as known in the art. The C10 or C7-hydroxylated monoterpene product may be isolated from the culture or culture medium (which may for example be a large scale batch or continuous culture fermenter) using methods known in the art, such as using solid phase extraction, chromatography methods, solvent extraction methods, distillation, etc. Purification methods obviously also depend on whether the product is present into the culture medium or whether it is present within the host cells. For clinical applications the product is purified to high purity levels of e.g. 90%, 95%, 99% purity.

In one embodiment cytochrome P450 genes or enzymes endogenous to the host cell are inactivated (e.g. by mutagenesis) to avoid non-specific conversion of the substrate. Likewise, other endogenous enzymes, or genes encoding these, may be inactivated if they interfere with the production of the specific product desired.

It is understood that other nucleic acid sequences may be co-expressed in the host cell. As mentioned, it may be desired to co-express a CPR gene, a terpene synthase, such as limonene synthase (for perillyl alcohol production) or an α-pinene synthase (for myrtenol production), GPP-synthase or genes encoding additional isoprenoid biosynthetic enzymes in the same cell. In summary, the bioconversion method according to the invention comprises the steps of: making a recombinant host cell which produces a PINH enzyme according to the invention, culturing the host cell under appropriate conditions, either in the presence of a suitable monoterpene substrate (preferably limonene) or in the presence of suitable substrate precursors, and purifying the C10 or C7-hydroxylated monoterpene product (preferably perillyl alcohol) from the cell culture or culture medium.

The recombinant cells are preferably grown in large scale (industrial scale) cultures, such as batch fermentors.

It is understood that the invention encompasses the recombinant host cells or organism as described in any of the embodiments, as well as any derivatives thereof, such as obtained by multiplication of the cells, further modification of the cells or organism, by breeding with the recombinant organism, any tissues derived from the recombinant organism (seeds, pollen, fruit, etc.) and the like.

The substantially purified (+) or (−)-perillyl alcohol may then be used as chemoprotective or chemotherapeutic drug (for prophylaxis or therapy of cancers, especially solid cancers), as a drug for the treatment or prophylaxis of angiogenesis dependent diseases such as solid tumors and hematopoietic malignancies (see Loutrari et al. 2004, J. Pharmacol Exp Ther June 21 epublication) or as an additive to food/feed products. Thus pharmaceutical compositions according to the invention comprising a suitable amount of the PINH hydroxylation product, obtainable by the described method, are manufactured as known in the art. The composition may comprise various other components, such as but not limited to water, saline, glycerol or ethanol and additional pharmaceutically acceptable auxiliary substances may be present, such as emulsifiers, wetting agents, buffers, tonicity adjusting agents, stabilizers and the like, for example, sodium acetate, sodium lactate, sodium chloride, potassium chloride, calcium chloride, sorbitan monolaurate, and triethanolamine oleate. Also other biologically effective compounds may be present, e.g. limonene. The compositions are preferably administered orally. For skin cancer treatment or prophylaxis the composition is suitable for topical application (see Gupta and Myrdal 2004, Int. J. Pharm. 269(2):373-383). Pharmaceutical dosage forms may be tablets, powders, gels, liquids, etc. Further guidance regarding formulations that are suitable for various types of administration can be found in Remington's Pharmaceutical Sciences, Mace Publishing Company, Philadelphia, Pa., 17th ed. (1985). The daily effective dosage of perillyl alcohol will vary, but may be in the range of about 10 or 20 mg per day for a 70 kg person up to 200, 300, 500, 1000 mg per day or more. A skilled person can determine what the most effective dosage is, which has the least side effects, such as nausea. As the in vivo half life of perillyl alcohol and metabolites thereof is short, a frequent dosage scheme (e.g. 2, 3, 4 or 5 timer/day) is preferred.

If food grade host cells (e.g. microorganisms having GRAS status or edible plant or animal cells/tissues) are transformed, the recombinant host cells which produce perillyl alcohol (and/or derivatives thereof) may also be used to manufacture food products or food additives having substantial health benefits, e.g. anti-carcinogenic properties when regularly ingested or inhaled by human or animal subjects. Thus, cells producing or having the capability to produce high amounts of perillyl alcohol may for example be harvested and used in the production of food products such as milk, butter, yoghurt, drinks, etc. Similarly animal feed with cancer protective properties may be made. Other products include cigars or cigarettes made from recombinant tobacco cells or plants producing and accumulating perillyl alcohol, wherein such cigarettes or cigars are healthier than traditional products. The lung cancer incidence and/or severity may thereby be reduced. Such plants can be made as described above.

In another embodiment, the aromatic toluene may be used as a substrate to produce benzylalcohol, a colorless liquid with weak, slightly sweet odour and constituent of many essential oils both free or as ester that is used in perfumery and flavour industries and as an anti-microbial preservative in pharmaceuticals and cosmetics. Benzylalcohol may be further oxidised either by microbial enzymes or with industrial enzymes or chemically to benzaldehyde which has a bitter, almond like taste and is used extensively in the flavour industry. Also, benzylalcohol can be esterified to a whole series of esters that are used extensively in the flavour and fragrance industry, such as benzyl acetate, benzyl propionate and benzyl isovalerate.

### 5. Gene Therapy Applications Using PINH Encoding Nucleic Acid Sequences

Gene therapy is an attractive alternative to continuous drug delivery and especially in cancer gene therapy and angiogenic gene therapy significant advances have been made. “Gene therapy” can be defined as the introduction of nucleic acid sequences into target cells of the human body, whereby an active molecule (e.g. antisense or siRNA or a protein) is made at the target site. The target cells may be tumor cells (direct targets) or cells supporting tumor development and/or spread, such as tumor endothelial cells (indirect targets). The patient essentially makes the therapeutic molecule continuously in vivo in the target cell.

The most limiting factor in gene therapy to date is the gene delivery system or “vector” (the gene delivery vehicle). A number of different viral and non-viral vectors have been developed, which will be shortly described below. A distinction is also made between “in vivo” and “ex vivo” gene delivery. In vivo gene delivery refers to the introduction of the nucleic acid into the target cell in vivo, i.e. in the human body. In contrast, “ex vivo” gene delivery refers to the delivery of the nucleic acid sequence into cells removed from the human body. The cells comprising the nucleic acid sequence are then re-introduced into the body.

In one embodiment according to the invention the PINH encoding DNA sequence is used in cancer and angiogenic gene therapy, in order to produce perillyl alcohol within target cells in the human body, i.e. in vivo. Provided are various gene therapy vectors and methods, as well as ex vivo recombinant human cells comprising the PINH encoding DNA sequence. As it is necessary to provide the PINH substrate limonene either in or to the same target cells, the gene therapy methods involve the delivery and expression of the following coding sequences within the target cell: 1) a GPP-synthase coding sequence, 2) a limonene synthase coding sequence, 3) a PINH coding sequence and optionally 4) an NADPH cytochrome P450 reductase (unless the target cells already provide this activity). In another embodiment, the substrate limonene is provided to the target cells orally or by injection. In this case the method comprises delivery and expression of the following genes within the target cells: 1) a PINH coding sequence and optionally 2) an NADPH cytochrome P450 reductase (unless the target cells already provide a suitable electron donor). Suitable coding sequences of any of these genes are those already described elsewhere herein.

“Gene delivery” or “gene transfer” refers herein to methods for reliable introduction of recombinant or foreign DNA into host cells. The transferred DNA can remain non-integrated or preferably integrates into the genome of the host cell. Gene delivery can take place for example by in vivo or ex vivo transduction, using viral vectors, or by transformation of cells ex vivo. “Transduction” refers to the delivery of a DNA molecule into a recipient host cell by a virion, leading to a “transduced” host cell containing the recombinant vector which was in the virion. “Host cell” or “target cell” refers to the cell into which the DNA delivery takes place, such as the cancer cell of a subject. The host cell may be present within the human body or in cell culture.

The invention encompasses both non-viral and viral gene delivery vectors, although viral vectors are preferred. “Non-viral vectors” are either a) naked expression vector DNA comprising the three chimeric genes under suitable promoters (promoters active constitutively, or preferably active only in the target cells) or b) cationic liposomes comprising such expression vector DNA. To facilitate uptake into the cell as endosomes, targeting proteins such as antibodies have been included in liposomes (Marty et al. 2002, Br J Cancer 87:106-112). Naked DNA (in the form of a plasmid) can be directly injected (Wolff et al, 1990, Science 247: 1465-1468.) or attached to gold particles that are bombarded into the tissue (Cheng et al, 1993, Proceedings of the National Academy of Sciences of the U.S.A. 90: 4455-4459.). For further details regarding the design and use of these non-viral vectors see Tandle et al. 2004, Journal of Translational Medicine 2:22, pages 10 and 11 and the references referred to therein. Various “viral vectors” have been developed, based on mainly five different viruses: adenoviruses (Ad), adeno-associated viruses (AAV), retroviruses, lentiviruses and herpex simplex-1 viruses (HSV-1) (see Thoma et al. 2003, Nat Rev Genet. 4:346-358 for a review). Retroviruses and lentiviruses can integrate into the cell genome, while the other vectors mainly remain episomally in the host cell (although AAV vectors have also been shown to integrate in some tissues into the genome of the target cell, see Hirata et al. 2000, J. of Virology 74:4612-4620). The episomal nature limits the duration of the gene expression, making these vectors less suitable for long-term expression. On the other hand can the non-integrating vectors transfect both dividing and non-dividing cells, while the integrating vectors only infect dividing cells. Each vector system has its own advantages and disadvantages.

In the present invention any one of the existing viral vectors may be used to deliver the genes encoding the three enzymes above into target cells, especially into cancer cells such as cells of solid tumors (mammary-, liver-, pancreatic-, colorectal-, ovarian-, prostate-, etc.). A skilled person will know which viral vector is the most appropriate to use and can use known molecular and virology techniques to construct such a vector.

Viral vectors used for in vivo gene therapy (systemic or local injection) can be specifically targeted to the tumor cell directly or to related cell (e.g. endothelial cell) using known methods, such as modification of the viral capsid protein to incorporate an ligand recognized by the a surface receptor on the target cell (resulting in internalization mediated by ligand-receptor interaction) or by conjugation of antibodies to the viral capsid, which specifically recognize target cell surface receptors.

A further (additional) targeting mechanism is to use target-cell or tissue specific promoters, so that delivery to a non-target cell does not result in expression of the operably linked sequence (target-tissue specific expression, especially in cancer cells). For EC targeting for example promoters for endoglin, endothelin-1 gene (Varda-Bloom et al. Gene Ther. 2001, 8:819-827) or von Willebrand factor may be used. Prostate cancer-specific promoters are for example the long PSA and osteocalcin (Shirikawa et al. 2000, Mol. Urol. 4(2):73-82). Tumour specific promoters include the tyrosine kinase promoter for B16 melanoma (Diaz et al, 1998, Journal of Virology 72: 789-795), DF3/MUC1 for certain breast cancers (Wen et al, 1993, Nucleic Acids Research 21: 1911-1918) and afetoprotein for hepatomas (Chen et al, 1995, Journal of Clinical Investigation 96: 2775-2282). A range of promoters exist which are only active in specific tissue such as the liver (albumin promoter Miyatake et al, 1997, Journal of Virology 71: 5124-5132), muscle (myosin light chain 1; Shi et al, 1997, Human Gene Therapy 8: 403-410.) and endothelial cells (von Willebrandt promoter, Ozaki et al, 1996 Human Gene Therapy 7: 1483-1490; smooth muscle 22a promoter, Kim et al, 1997, Journal of Clinical Investigation 100: 1006-1014). The temporal expression of the transgene construct can be controlled by drug inducible promoters, for example by including cAMP response element enhancers in a promoter, cAMP modulating drugs can be used (Suzuki et al, 1996) or tetracycline inducible promoters. Alternatively repressor elements (e.g. Tet repressor) can prevent transcription in the presence of the drug (Hu et al, 1997, Cancer Research 57: 3339-3343). Similarly radiation induced promoters may be used (Hallahan et al. 1995, Nature Medicine 8: 786-791). Obviously, a range of other suitable promoters exist. Mostly viral promoters are used, although human promoters are also available.

Provided is thus a viral vector comprising the coding sequences of a PINH enzyme and optionally one or more additional coding sequences. Each of the coding sequences may be under the control of separate promoter sequence active in the target cell. Alternatively, the vector may be a polycistronic vector, such as a tricistronic vector, whereby a single promoter is operably linked to the coding sequences, which are separated by IRES elements (Internal Ribosome Entry Sites). The single RNA transcript is translated into the three heterologous gene products within the transfected cell. Such vectors have already been described for retroviral vectors, see Douin et al. 2004 (BMC Biotechnology 4:16). Suitable IRES elements are available, such as the IRES from EMCV.

The recombinant viral vector is packaged into virion particles, which are used to transfect human cells in vitro (cell cultures e.g. cancer cells removed from the subject) or, preferably, in vivo. In vivo transfection involves the administration (systemic or local injection) of the virions to the subject. Ex vivo transduced cells are administered to the subject by for example injection, reinplantation or reinfusion. Ex vivo transduced cells may optionally be selected prior to administration, e.g. the mRNA expression and perillyl alcohol production may be determined and high expressing cell lines selected. Perillyl alcohol levels may for example be determined using HPLC.

Administration dosages will vary. A skilled person can easily determine the therapeutically effective amount by routine trial and error and by e.g. drawing dose-response curves. For rAAV virions (recombinant AAV) typically dosages are at least 10³ to 10⁵ rAAV virions, preferably at least 10⁷ or 10⁸ virions, more preferably 10⁹ to 10¹¹ virions or more. Optionally administration is repeated at later stages. As mentioned above, the substrate limonene may be administered separately, locally or systemically, either prior, concomitantly or subsequently to administration of the recombinant virions.

The virions transfect the target cells and deliver the vector into the target cell(s). Preferably, the vector is stably integrated into the genome of the transduced cell and provides expression of the proteins and especially leads to the production of perillyl alcohol in the cells. In vivo expression is preferably high enough to be therapeutically effective, e.g. to result in tumor regression. The therapeutically effective amount will depend on the tumor type and can be regulated by choosing appropriate promoters.

Thus, the invention provides in one embodiment a method for delivering a nucleic acid molecule to cancer cell in vivo or ex vivo, the method comprising the steps of (a) providing a recombinant virion, wherein the virion comprises a vector, the vector comprising one or more expression elements operably linked to a nucleic acid sequence encoding a functional PINH enzyme and optionally one or more other enzymes, especially a functional limonene synthase enzyme and a functional GPP synthase enzyme and/or a functional electron donating protein; and, (b) bringing the virion into contact with the target cell, whereby transduction of the vector results in expression of the nucleic acid sequence(s) in the transduced target cells, leading to production of perillyl alcohol in the transduced cells, either following external supply of PINH substrate to the cells or following endogenous substrate production.

Provided are, therefore, recombinant gene therapy vectors and recombinant virions comprising a nucleic acid sequence encoding a PINH enzyme according to the invention, transfected mammalian cells (ex vivo and/or in vivo) comprising said nucleic acid sequence(s), pharmaceutical compositions comprising suitable amounts of recombinant virions, methods for delivering a PINH encoding nucleic acid sequences to mammalian cell, as well as method for producing perillyl alcohol in mammalian cells ex vivo and in vivo.

The PINH gene and optionally one or more other genes may also be introduced into recombinant mammalian cells using artificial human chromosomes, as described in the art.

### Sequences

SEQ ID NO 1: Fragaria x ananassa monoterpene hydroxylase cDNA
SEQ ID NO 2: Fragaria x ananassa monoterpene hydroxylase coding region
SEQ ID NO 3: Fragaria vesca monoterpene hydroxylase coding region
SEQ ID NO 4: Fragaria x ananassa monoterpene hydroxylase protein
SEQ ID NO 5: Fragaria vesca monoterpene hydroxylase protein

### FIGURE LEGENDS

FIG. 1

Compartmentation of isoprenoid biosynthesis in the plant cell. The mevalonate pathway is active in the cytosol (and supplies IPP to mitochondria) while the MEP (methylerythritol 4-phosphate) pathway is active in plastids. Enzymatic steps similar in both the cytosolic and plastidic pathways are represented in the common area. IPP, isopentenyl diphosphate; DMAPP, dimethylallyl diphosphate; GPP, geranyl diphosphate; FPP, farnesyl diphosphate; GGPP, geranylgeranyl diphosphate.

FIG. 2

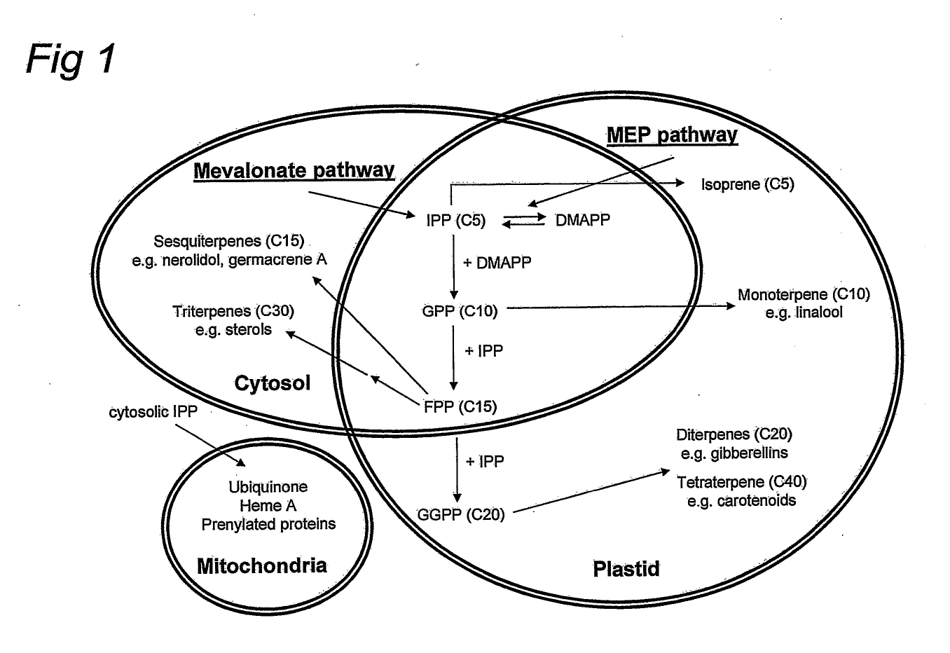

Terpenoid production in wild and cultivated strawberry species.

(A) Terpenoids detected by headspace analysis of ripe fruits. GC-MS chromatograms (selected m/z 93) after headspace Tenax trapping (see methods) showing the different terpenes emitted by cultivated (top) and wild (bottom) ripe strawberry fruit. A trace of the monoterpene alcohol myrtenol was also detected in wild strawberry (data not shown).

(B) Reactions catalyzed by terpene synthases (TPS) for the formation of the monoterpene alcohol linalool and the sesquiterpene alcohol nerolidol.

(C) Reactions catalyzed by a terpene synthase (TPS) enzyme for the formation of the monoterpene α-pinene, a cytochrome P450 enzyme catalyzing a subsequent hydroxylation step at C10 forming myrtenol, and an alcohol acyltransferase (AAT) forming myrtenyl acetate.

FIG. 3

A,B, Production of myrtenol from α-pinene by recombinant FaPINH. Microsomes isolated from yeast harboring either the empty vector (A) or the vector containing the FaPINH coding region (B) were used for enzymatic assays using (−)-α-pinene as a substrate. Total ion chromatograms from the GC-MS analysis of the products are shown.

C-J, Hydroxylation of alternative substrates by recombinant FaPINH. C,D limonene; E,F α-phellandrene; G,H α-terpinolene; I,J α-terpinene. In each pair, the first panel shows the results of the empty vector, the second panel with the vector harboring FaPINH.

FIG. 4

Reaction schemes of the hydroxylation of terpenoid substrates as demonstrated in FIG. 3.

FIG. 5

Likely other candidate substrates for FaPINH. Arrows indicate the expected hydroxylation site

FIG. 6

Expression analysis of the FaPINH gene in different tissues of cultivated (top pair of blots) and the wild strawberry in leaf, root and red ripe fruit tissues (bottom pair of blots). The entire FaPINH cDNA was used to hybridize the RNA gel blots. A ribosomal RNA (rRNA) probe was used as a control for equal loading.

FIG. 7.

Effects of different concentrations of linalool (∘), myrtenol (Δ), perilla alcohol (□) and perilla aldehyde (⋄) on spore germination of Phythophthora infestans (A) and Botrytis elliptica (B)

FIG. 8

Effects of different concentrations of linalool (∘), myrtenol (Δ), perilla alcohol (□) and perilla aldehyde (⋄) on mycelial growth of Botrytis elliptica (A), Phythophthora infestans (B) and Pythium aphiadermatum (C).

FIG. 9

Diagram describing the four different constructs that were used for the transformation of potato.

The following non-limiting Examples illustrate the different embodiments of the invention. Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, and Sambrook and Russell (2001) Molecular Cloning: A Laboratory Manual, Third Edition, Cold Spring Harbor Laboratory Press, NY; and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK.

### EXAMPLES

### Example 1

### General Material and Methods

### 1.1 Plant Material

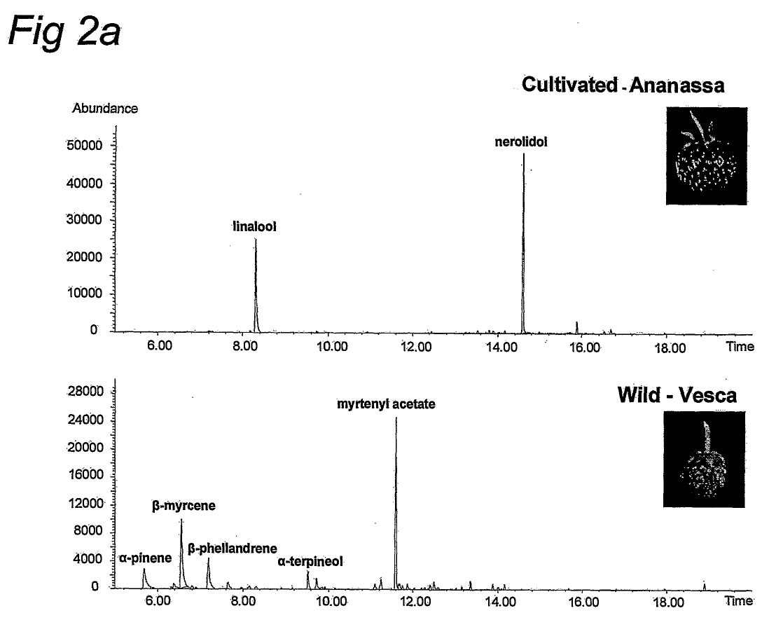

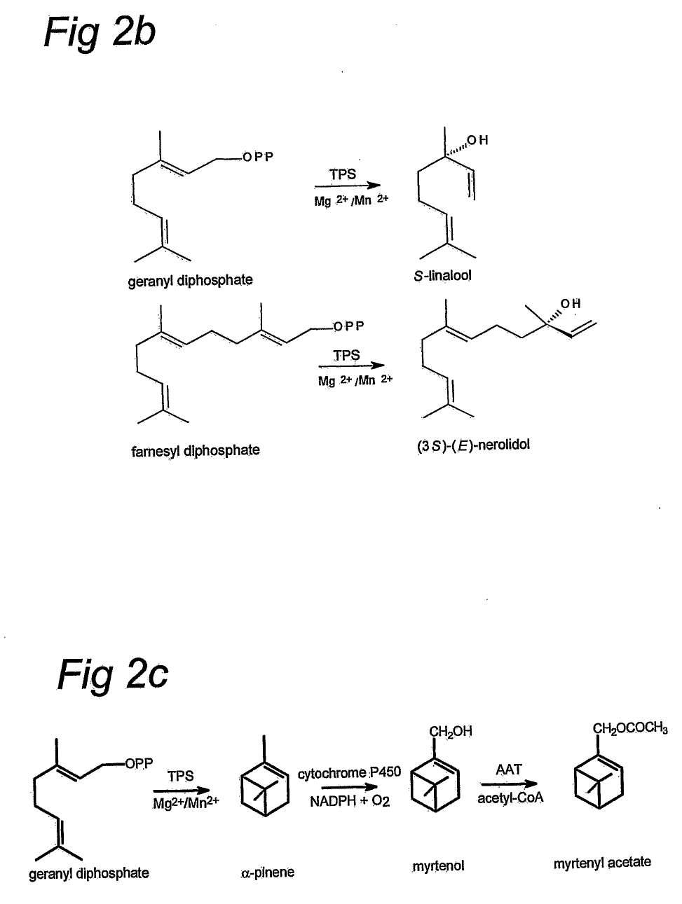

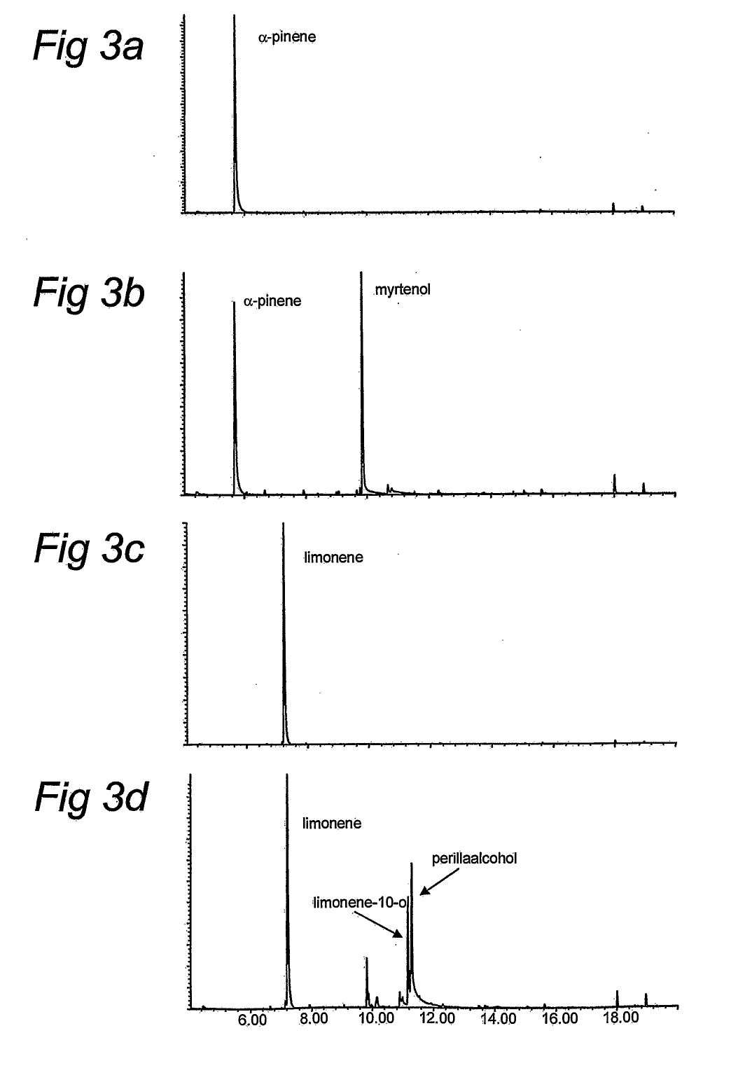

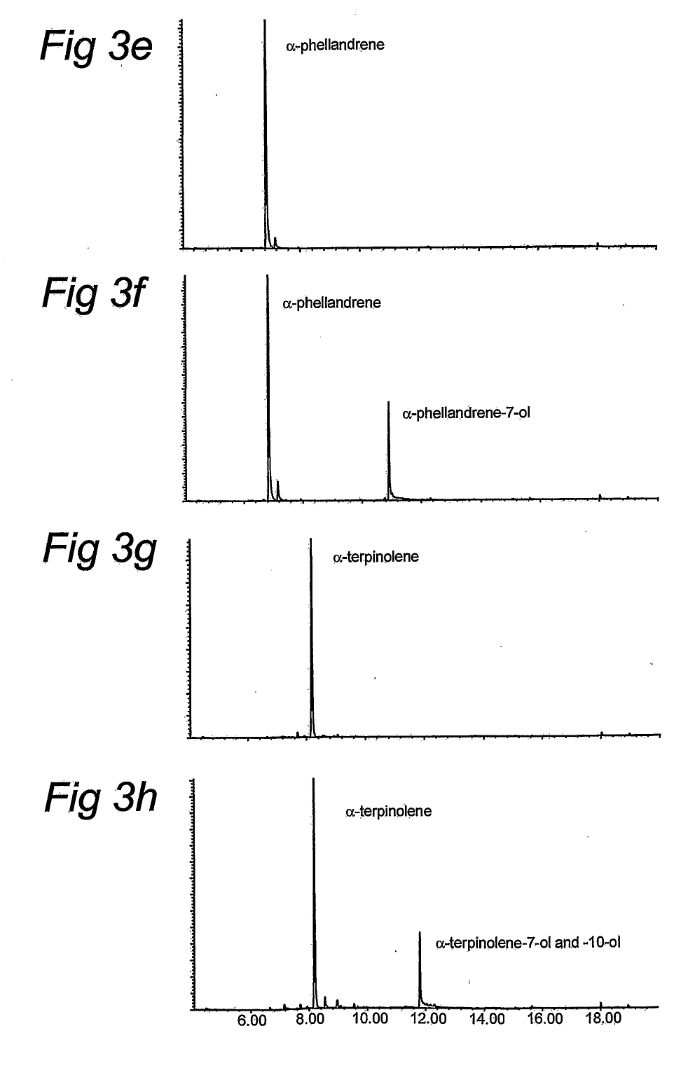

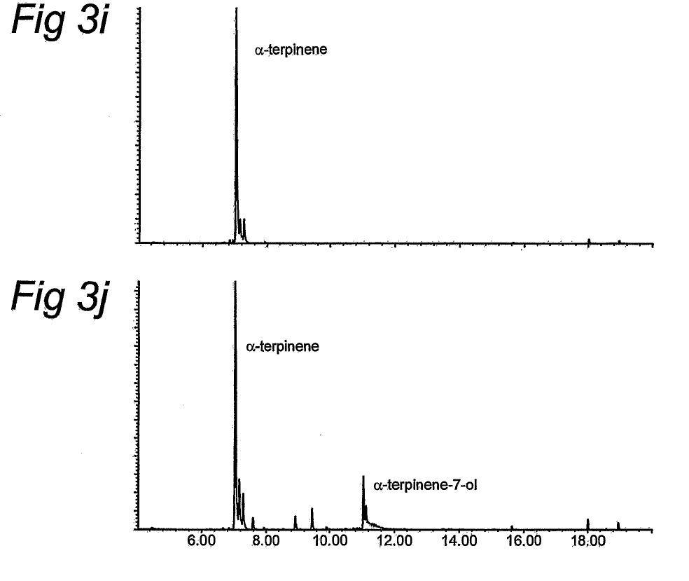

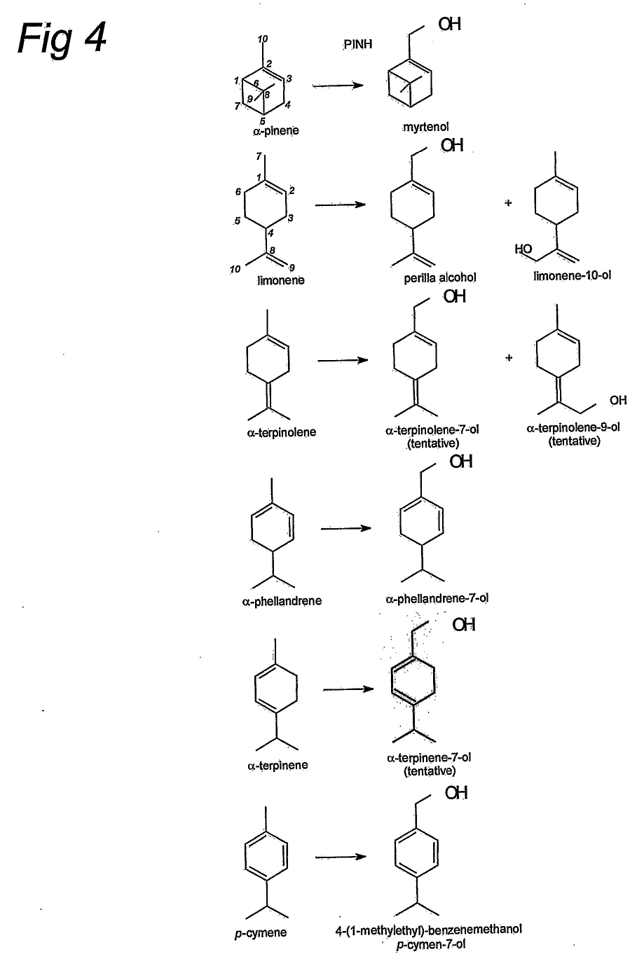

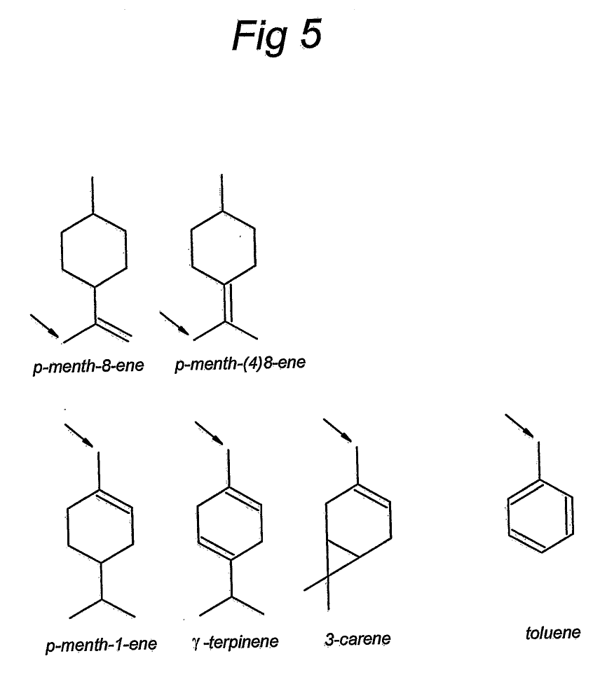

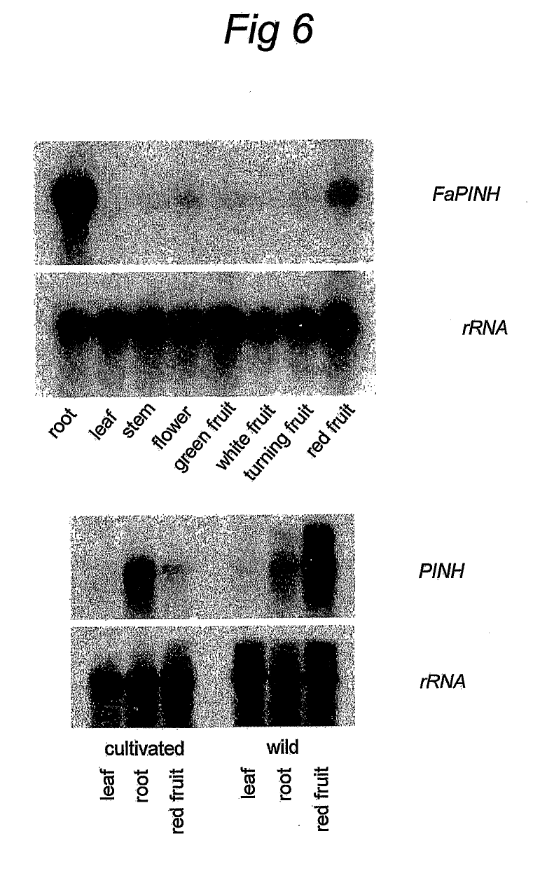

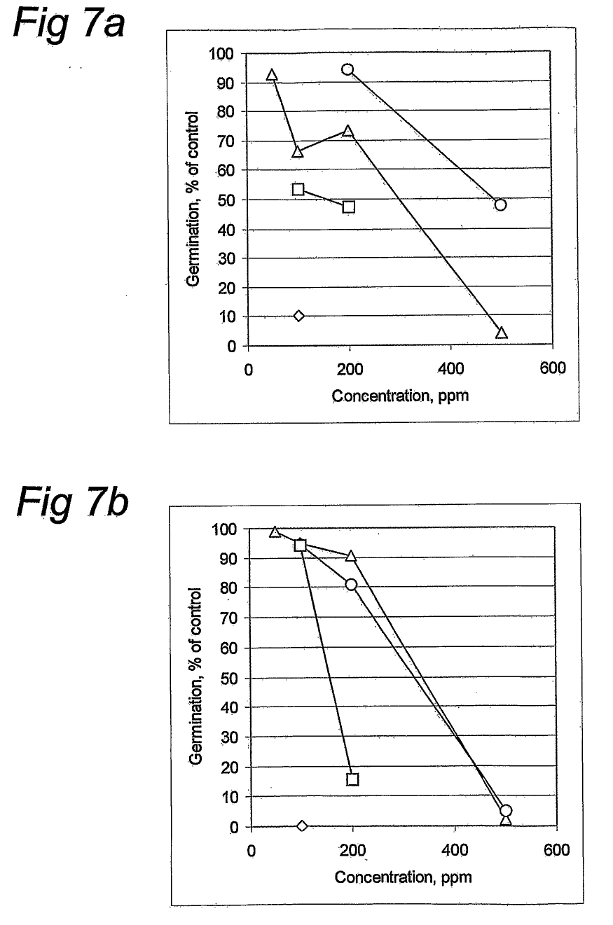

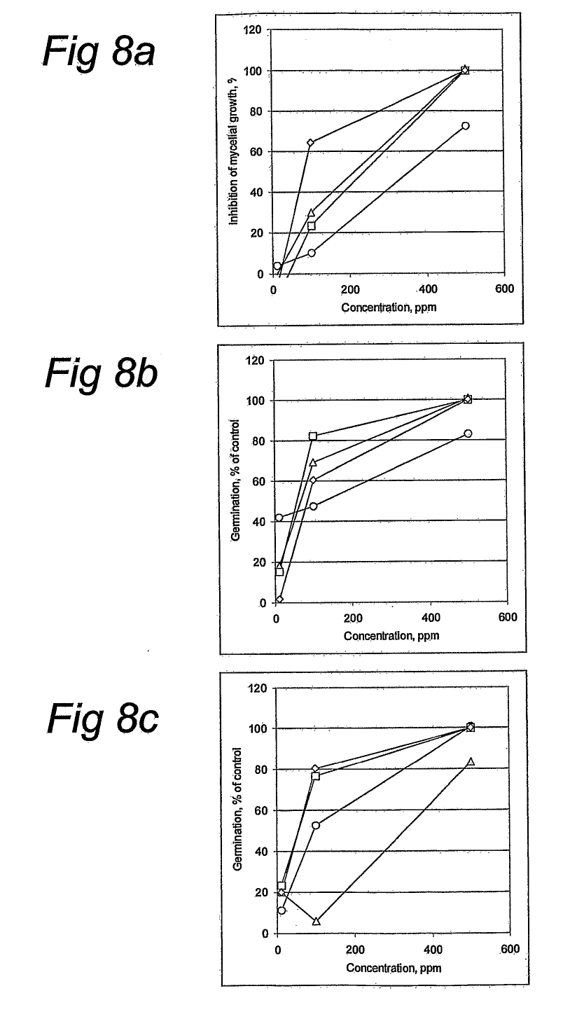

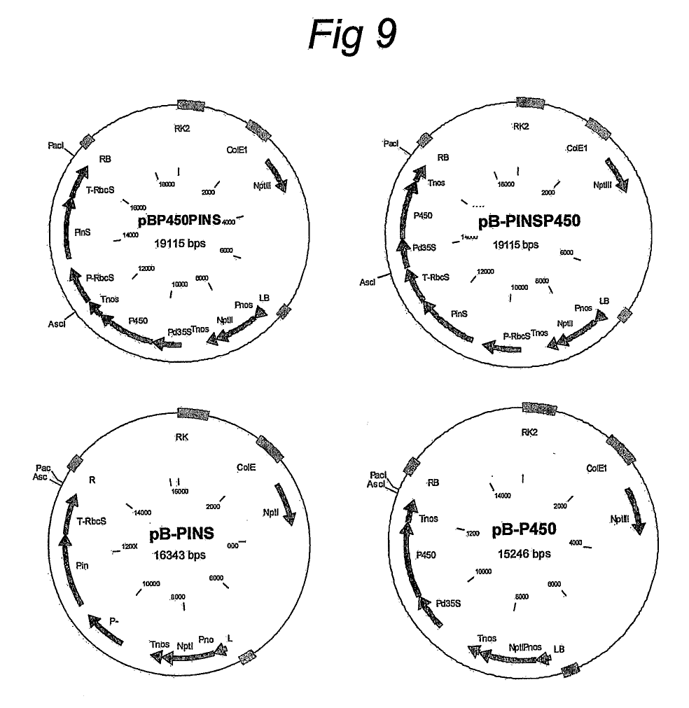

Greenhouse-grown strawberry varieties and lines of wild species from the Plant Research International (PRI) breeding collection were used. Volatile analysis (FIG. 2A) was conducted using Elsanta as the cultivated variety and PRI accession 92189 as the wild species. For RNA gel-blots, the Elsanta cultivar (FIGS. 3B, 3E and 3F), the PRI accessions H1 and 92189 as wild species (W) and Gorella and Holiday as cultivated forms (CU; in both FIG. 3C and FIG. 3D) were used. PCR on genomic DNA and expression analysis using RT-PCR (FIG. 6) were carried out using CU1 (cv. Sure crop), CU2 (cv. Holiday), CU3 (cv. Senga sengana), CU4 (cv. Gorella), CU5 (cv. Calypso), CU6 (cv. Elsanta), CU7 (PRI accession 75169), and WI1 (PRI accession FA-1), WI2 (PRI accession FA-2), WI3 (PRI accession FA-3), WI4 (Yellow wonder), WI5 (Alexandria), WI6 (PRI accession 92189), and WI7 (PRI accession H2).

### 1.2 Analysis of Fruit Volatiles

For the purpose of headspace analyses, red ripe strawberry fruits were enclosed in 0.7 L glass jars fitted with a teflon-lined lid equipped with an inlet and an outlet. A vacuum pump was used to draw air through the glass jar at approximately 100 mL min-1, the incoming air being purified through a glass cartridge (140×4 mm) containing 150 mg Tenax TA (20/35 mesh, Alltech, Breda, the Netherlands). At the outlet the volatiles emitted by the detached fruits were trapped on a similar Tenax cartridge. Volatiles were sampled over 24 h. Cartridges were eluted using 3×1 mL of redistilled pentane-diethyl ether (4:1). Of these samples, 2 μL was analyzed by GC-MS, using an HP 5890 series II gas chromatograph equipped with an HP-5MS column (30 m×0.25 mm i.d., 0.25 μm d.f.) and an HP 5972A Mass Selective Detector as described by Bouwmeester et al, (1999, Plant Physiol 121, 173-180).

### 1.3 Solid-Phase Extraction

The XAD-2 column was purchased from Supelco, Bellefonte, USA. Myrtenol, phenol, methanol, and diethyl ether were obtained from Aldrich, Deisenhofen, Germany. Samples were stored at −20° C. until work-up. Frozen samples were weighed and submerged in an equal volume of water, homogenized by means of an Ultra-Turrax and centrifuged (2000 g; 10 min). The pellets were washed twice, the supernatants were combined (approx. 40 ml) and subjected to solid-phase extraction on XAD-2 (20 cm, 1 cm i.d). The XAD-2 column was preconditioned with 50 ml methanol and 100 ml water. After application of the sample, the XAD-2 was successively washed with 50 ml water, 50 ml diethyl ether and 80 ml methanol. The diethyl ether extract was dried over sodium sulfate and concentrated to approx. 100 μl. Phenol (0.1 mg/ml) was added as an internal standard. The methanol extract was concentrated in vacuo to approx. 1 ml.

### 1.4 Enzymatic Hydrolysis and GC-MS Analysis

Enzymatic hydrolysis was performed by dissolving an aliquot of the methanol extract, as described above, in 2 ml 0.2 M phosphate buffer pH 5.5. The solution was extracted twice with the same volume of diethyl ether to remove free alcohols. Subsequently, 200 μl Rohapect D5L (Röhm) were added, a pectinolytic enzyme preparation exhibiting glycosidase activity. After an incubation period of 24 h at 37° C., the liberated aglycons were extracted twice with 1 ml of diethyl ether. The combined organic layers were dried over sodium sulfate and concentrated.

Capillary gas chromatography-mass spectrometry (GC-MS) analysis was performed with a Fisons Instruments (Fisons, Engelsbach, Germany) GC 8000 Series, coupled to a Fisons Instruments MD800 quadrupol mass detector fitted with a split-injector (1:20) at 230° C. A DB-Wax fused silica capillary column (30 m×0.25 mm i.d.; df=0.25 μm) (J & W, Folsom, Calif., USA) was used with a run program: from 50° C. for 3 min to 220° C. for 10 min, with a temperature increase of 4° C. min-1, using a 2 ml·min-1 helium gas flow rate. The Xcalibur for Windows software was used for data acquisition. The significant MS operating parameters were: ionization voltage 70 eV (electron impact ionization); ion source temperature 220° C.; interface temperature 250° C. Constituents were identified by comparing their mass spectra and retention indices with those of authentic reference compounds. Multidimensional gas chromatography-mass spectrometry (MDGC-MS) on a Fisons 8160 GC connected to a Fisons 8130 GC and a Fisons MD 800 quadrupole mass spectrometer was used to analyse the absolute configuration of α-pinene as described by Lücker et al. (2002).

### Example 2

### Isolation and Characterisation of PINH

By mining an EST sequence collection generated by random sequencing of a cultivated strawberry ripe fruit cDNA library (Aharoni and O'Connell, 2002), five different EST clones were identified which showed homology to cytochrome P450 genes cloned from a vast number of other organisms. Protein sequence alignment to published cytochrome P450s showed that, of these five, the D59 clone is related to the CYP71 family. This family of cytochrome P450 proteins has previously been shown to be associated with monoterpenes metabolism (Hallahan et al., 1994, Bioch. Biophys. Acta 1, 94-100; Lupien et al., 1999, Arch Bioch Biophys 368: 181-192; Bertea et al, 2001, Arch Bioch Biophys 390: 279-286).

Detailed gene expression analysis using the five different fragments as probes for RNA gel-blot hybridizations revealed that clone D59 showed increased expression in the ripe red strawberry fruit, but was also expressed, to even higher levels, in roots. The protein putatively encoded by the D59 gene showed the highest homology (49%-50% identity) to three Arabidopsis proteins with unknown functions (CYP71A26, CYP71A25 and CYP71A22). Cloning of the corresponding gene from the wild species (termed FvPINH) showed that the proteins from the wild and the cultivated species differed by only three amino acid residues (data not shown).

PINH gene expression was further analyzed in leaf, root and ripe fruit tissues of wild and cultivated species (FIG. 6). The results show that PINH is expressed at high levels in ripe fruit of the wild species, higher than in ripe fruit of the cultivated species. Expression of PINH was detected in roots of both strawberry species (though at higher levels in the cultivated species), while only very low levels could be detected in leaves of both species.

The construct for expression in Saccharomyces cerevisiae was generated by amplifying the entire FaPINH coding region by PCR with Pfu DNA polymerase using oligonucleotides that introduced a BglII restriction site upstream of the start codon and an EcoRI site downstream of the stop codon. Oligonucleotides used were:

(AAP113: 5′-CAGATCTATGGAAGCCACTTCTTGGGTTAC 3′)
(AAP114: 5′-CCTTAAGAGAAGCTAGTAGCTGGAACC 3′).

PCR products digested with BglII and EcoRI were ligated to the pYeDP60 expression vector (Pompon et al., 1996, supra) which was digested with BamHI and EcoRI in between the artificial promoter GAL10-CYC1 and the PGK terminator. Recombinant pYeDP60 plasmids were transferred into Saccharomyces cerevisiae cells using the lithium acetate method (Ito et al., 1983, J Bacteriol 153: 163-168). The yeast host cells employed were WAT11U and WAT21U (Pompon et al., 1996, Method Enzymol 272: 51-64). Both strains contain an insertion in the endogenous NADPH-cytochrome P450 reductase locus (CPR1) which is replaced by either ATR1 or ATR2, the two NADPHcytochrome P450 reductase genes from Arabidopsis thaliana (Urban et al., 1997, supra).

Transformants were selected on SGI medium, while expression was carried out in YPL medium (induction with 2% galactose) (Pompon et al., 1996, supra). Samples of harvested yeast cells were assayed spectrophotometrically for cytochrome P450 content as detected by CO binding spectra (Omura and Sato, 1964, J Biol Chem 239: 2370-2378).

Transformants containing significant cytochrome P450 levels as well as control yeast cells harboring the expression vector without insert were also used to prepare microsomes by means of published procedures (Pompon et al., 1996, supra). Microsome preparations were evaluated by CO-difference spectrum and were assayed for (−)-α-pinene hydroxylation. The reaction mixture for the recombinant enzyme activity assay, in a final volume of 1 ml, contained 50 mM Tris-HCl pH 7.4, 1 mM EDTA, 0.1 mM DTT, 0.8 units glucose-6-phosphate dehydrogenase, 2 mM glucose 6-phosphate, 5 μM FAD, 5 μM FMN, 1 mM NADPH, and 200 μL of microsomal preparation. 200 nmol (−)-α-pinene (in less than 10 μl, which has no detectable influence on the reaction) were added to the mixture to start the reaction, which was allowed to proceed for 2 hours at 30° C. with gentle shaking. Microsomes isolated from cells harboring the empty vector were employed as controls. The reaction was stopped by chilling the mixture on ice and extracting twice with 1.0-mL portions of pentane:diethyl ether (4:1). The combined extract was passed through a short column of silica gel and anhydrous MgSO₄, after which the column was washed with 1.5 mL diethyl ether to remove residual compounds. After concentration under N2, the samples were analyzed by GC-MS. Samples (2 μL) were analyzed by an automated injector on a Hewlett-Packard 5890 series II gas chromatograph equipped with a 30 m×0.25 mm inner diameter fused silica column coated with a 0.25 μm film of HP 5MS (Hewlett-Packard), and a Hewlett-Packard 5972A Mass Selective Detector. GC oven temperature was programmed at an initial temperature of 45° C. for 1 min, with a ramp of 10° C. min-1 to 280° C., and a final time of 10 min. Full spectra were recorded for major reaction products, which were identified by comparing retention times with authentic standards and by comparing spectra with those of the NSB75K library, using the G1033A NIST probability based matching algorithm. The identity of the product was confirmed by coincidence of retention time with the authentic standard.

Assays with microsomes isolated from FaPINH expressing yeast showed that the substrate α-pinene was highly efficiently (over 50%) hydroxylated at C-10 to form myrtenol (FIGS. 3A,B). The (−)-α-pinene form was preferred to (+)-α-pinene as a substrate. In this reaction also some myrtenol was produced probably as a result of endogenous, α-specific, oxidising E. coli enzymes.

Then the production of myrtenol in fruit and roots of various wild and cultivated strawberry species was analyzed. The free form of myrtenol was detected in ripe fruit of four wild species, but not in any of the eight cultivated species examined (Table 1). The same pattern was detected for glycosylated myrtenol and myrtenyl acetate. On the other hand, relatively high levels of free and glycosylated myrtenol (more than in ripe fruit tissue of the wild species) were detected in the roots of both species.

### Example 3

### Substrate Specificity of PINH

PINH catalytic activity was also tested with a range of other substrates with similar structure. Despite the reported high substrate specificity, a range of other substrates were hydroxylated by PINH. For example, limonene that has an allylic C7, analogous to the α-pinene C10, is efficiently hydroxylated to perilla alcohol (FIG. 3D, FIG. 4). Other substrates that were hydroxylated at the same position were α-phellandrene, α-terpinolene and α-terpinene (FIG. 3 F,H,J; FIG. 4). In addition, the α-phellandrene, α-terpinene and limonene substrates used contained a trace impurity of p-cymene which was also hydroxylated at C7 yielding 4-(1-methylethyl)-benzenemethanol (p-cymen-7-ol) (FIG. 4). In addition, despite the reported high regio-selectivity of cytochrome P450 enzymes also some hydroxylation occurred in different regions. For limonene this second product peak could be identified as limonen-10-ol and for α-terpinolene also two product peaks were visible (in slower temperature program, not visible in FIG. 3) which were tentatively identified as the 7-OH and 10-OH alcohols (FIG. 4).

From the results it was deduced that this P450 is able to hydroxylate a variety of (mono)terpenoid at different positions. It is likely the P450 can also hydroxylate a range of other terpenoids at positions corresponding to C9 or C10 in α-terpinolene and limonene, respectively: for example p-menth-8-ene and p-menth-4(8)-ene (FIG. 5). Or at the position corresponding to C7 or C10 of limonene and α-pinene, respectively: for example p-menth-1-ene, γ-terpinene and 3-carene. Considering that also the aromatic p-cymene is hydroxylated it is likely that other aromatic compounds can be hydroxylated, for example toluene to yield benzyl alcohol.

### Example 4

### Antimicrobial Effects of Myrtenol perilla Alcohol etc.

One application for the finding is the production of hydroxylated monoterpenoids or aromatic compounds in transgenic plants (see Example 5) in order to obtain or enhance resistance against pests and/or pathogens. From the literature it is known that terpene alcohols, aldehydes and acids have bio-control activities in vitro.

Moreover, it has been shown before that hydroxylated monoterpenoids, like linalool, can be produced in transgenic plants and introduce resistance against plant pathogenic micro-organisms in these transgenic plants (WO 02/064764). Finally, it has been shown that it is feasible to introduce a 2-step biosynthetic pathway into plants consisting of a monoterpene synthase and a cytochrome P450 hydroxylase, and leading to the production of the expected hydroxylated monoterpene (Lücker et al., 2004. Plant Journal 39: 135-145) and Example 5.

To assess the bio-control activity of some of the hydroxylated compounds, which can be produced using the present invention, and/or products formed from these compounds either by endogenous or engineered enzymes, an in-vitro assay was used in which the inhibitory effect of said compounds against spore germination of the plant pathogens Botrytis elliptica and Phytophthora infestans and mycelial growth of Botrytis elliptica, Phytophthora infestans and Pythium aphanidermatum was analyzed. In order to estimate the possibilities of obtaining resistance in transgenic plants linalool was included as a reference compound. FIG. 7 shows that myrtenol, perilla alcohol and particularly perilla aldehyde are effective inhibitors of spore germination of B. elliptica and P. infestans. Against P. infestans, perilla alcohol and myrtenol are 2 to 3-fold more effective than linalool, perilla aldehyde even about 10-fold (FIG. 7A). Against B. elliptica perilla alcohol is about 2-fold more effective than linalool and myrtenol, whereas the concentration of perilla aldehyde tested (100 ppm) completely blocks germination (FIG. 7B).

Mycelial growth of the three plant pathogens was also effectively inhibited by myrtenol, perilla alcohol and perilla aldehyde (FIG. 8). For P. infestans, myrtenol, perilla alcohol and perilla aldehyde were more effective than linalool, with perilla aldehyde being even 4-5 fold more effective (FIG. 8A). For B. elliptica, myrtenol, perilla alcohol and perilla aldehyde were slightly more effective than linalool (FIG. 8B). For P. aphanidermatum, myrtenol was less effective than linalool, but perilla alcohol and perilla aldehyde were more effective (FIG. 8C). Considering the fact that good protection of transgenic plants producing linalool has been obtained (WO 02/064764) the results shown in FIGS. 7 and 8 indicate the high potential of compounds such as myrtenol, perilla alcohol and perilla aldehyde for obtaining resistance in transgenic plants.

Further conversion of the alcohols produced using PINH by endogenous plant or engineered enzymes to for example aldehydes and acids, or glycosylation enhances the protective effect. Aldehydes and acids may have a stronger antimicrobial effect than the alcohol. For example citral and geranic acid are more effective than geraniol (data not shown) and perilla aldehyde and perillic acid are more effective than perilla alcohol (FIGS. 7-8).

### Example 5

### Expression of PINH in Plants

In order to achieve expression of myrtenol and any of its derivatives in plants the presence of the α-pinene precursor is a prerequisite. Most plants do not produce α-pinene in significant amounts, and therefore the introduction of both α-pinene synthase and α-pinene hydroxylase is required for the synthesis of myrtenol in plants.

As an example potato plants were transformed with α-pinene synthase and α-pinene hydroxylase in two different way. The first experiment made use of cotransformation of two separate constructs carrying expression cassettes for the respective genes. In the second experiment both genes were combined on one T-DNA under the control of two different promoters.

### Constructs:

All constructs were made in pBIN++ plant expression vectors containing a double Asc-PacI acceptor site and based on pBINPLUS. For an overview of the constructs see FIG. 9.

The FaPinS gene was cloned into the rubisco gene expression cassette of ImpactVector1.4 (www.impactvector.com). The cDNA fragment encoding the mature protein was cloned behind the chloroplast targeting signal of the small subunit of rubisco using the multiple cloning sites NcoI-NotI. After shuttling the expression cassette with AscI-PacI into pBIN++, the resulting plasmid was called pB-PINS.

The FaPinH gene (named P450 in the diagrams below) was cloned in an expression cassette consisting of the double CaMV35S (Pd35S) promoter with AlMV enhancer and Nos terminator (Tnos). The cDNA was cloned using its own signals targeting it to the secretory pathway. After shuttling the expression cassette with AscI-PacI into pBIN++, the resulting plasmid was called pB-P450.

The genes were also combined on one T-DNA by cloning the expression cassettes into one pBIN++ vector. The genes were cloned in two different orders: LB-nptII-FaPinH-FaPinS-RB or LB-nptII-FaPinS-FaPinH-RB and named pB-P450PINS and pB-PINSP450 respectively.

### Plant Transformation

Potato was transformed using standard protocols (stem segment transformation). Table 3 shows the efficiency of obtaining transgenic plants using the different constructs and using different media. Expression of the genes caused a lowering of the transformation efficiency. After transfer to the greenhouse the highest expressors were reduced in growth and had a bleached phenotype.

TABLE 3
Table of the transformation success of potato explants with
using transformation on different types of media (TM).

| | | | | Number of | |
|---|---|---|---|---|---|
| | | | | explants | |
| | | | Number of | with Km- | |
| | | | explants | resistant | Efficiency |
| Constructs | Cultivar | TM | treated | shoots | % |
| pBPINS & | Kardal | ZCVK | 18 | 16 | 88.9 |
| pBP450 | | F-SIM | 37 | 35 | 94.6 |
| | | CHGRM | 33 | 19 | 57.6 |
| | RJ | ZCVK | 88 | 76 | 86.4 |
| | | F-SIM | 78 | 71 | 91.0 |
| | | CHGRM | 79 | 63 | 79.2 |
| pBPINS & | Kardal | ZCVK | 61 | 16 | 26.2 |
| pBDP450 | RJ | ZCVK | 211 | 76 | 36.0 |
| PBPINSP450 | Kardal | ZCVK | 45 | 34 | 75.6 |
| | RJ | ZCVK | 116 | 98 | 84.5 |
| pBP450PINS | Kardal | ZCVK | 45 | 33 | 73.3 |
| | RJ | ZCVK | 120 | 26 | 21.7 |
| pPBin++ | Kardal | ZCVK | 44 | 44 | 100 |
| | RJ | ZCVK | 40 | 40 | 100 |

TM: Transformation method
pBIN++ represents the empty transformation vector containing two sites for AscI-PacI subcloning. The empty vector has a higher transformation success than the different constructs.

Of all constructs 10-20 individual transgenic lines were analyzed using GC-MS headspace analysis using SPME and Tenax trapping (see e.g. Aharoni et al., 2003 (supra)). Controls transformed with just the α-pinene synthase produced large amounts of α-pinene. Lines transformed with the α-pinene synthase (under control of the Rubisco promoter) and subsequently with PINH under control of a double or single 35S promoter, produced α-pinene, myrtenol and myrtenol derivatives such as myrtenol glucoside. Also transformation with a double construct harbouring α-pinene synthase (under control of the Rubisco promoter) and PINH (under control of a double 35S promoter) resulted in transgenic lines producing α-pinene, myrtenol and myrtenol derivatives. In the latter lines α-pinene and myrtenol production were similar as that in the transgenic lines transformed with two different constructs. Selected transgenic lines of RJ and wildtype and empty vector controls were grown in the field and infection by P. infestans monitored. Table 4 shows that the three different strategies to obtain myrtenol production have all three worked and that these transgenic plants are protected against infection with P. infestans to varying degrees but even up to 100% in one case. In the same field experiment it was observed that some of the transgenic lines showed enhanced resistance against the phytophagous lady beetle, Epilachna vigintioctoinaculata.

TABLE 4
Infection by Phytophthora infestans of transgenic and wildtype/
empty-vector lines of potato cultivar RJ in the field.

| | | | alpha- | | | |
|---|---|---|---|---|---|---|
| Lines | Expression cassette | Phenotype | pinene¹ | NP | NSP | % SP |
| CK(NT) | Wild type | | — | 12 | 0 | 0.0 |
| CK(++) | Blank vector (pBin++) | | — | 8 | 0 | 0.0 |
| Z-16 | RBCS1 P/PINS + D35S P/P450 | | 9.9 | 10 | 0 | 0.0 |
| Z-23-1 | RBCS1 P/PINS + D35S P/P450 | Retarded | 13.8 | 4 | 1 | 25.0 |
| | | growth | | | | |
| Z-37-2 | RBCS1 P/PINS + D35S P/P450 | | 6.7 | 7 | 0 | 0.0 |
| Z-46-2 | RBCS1 P/PINS + 35S P/P450 | | 96.2 | 7 | 2 | 28.6 |
| F-19 | RBCS1 P/PINS + 35S P/P450 | Retarded | 1696.2 | 12 | 3 | 25.0 |
| | | growth | | | | |
| F-24-1 | RBCS1 P/PINS + 35S P/P450 | | 632.4 | 11 | 0 | 0.0 |
| F-32-2 | RBCS1 P/PINS + 35S P/P450 | | 232.9 | 5 | 5 | 100.0 |
| Z-11-1 | RBCS1 P/PINS//D35S P/P450 | Retarded | 181.9 | 9 | 0 | 0.0 |
| | | growth | | | | |
| Z-32-2 | RBCS1 P/PINS//D35S P/P450 | Retarded | 1640.1 | 1 | 0 | 0.0 |
| | | growth | | | | |
| Z-48-2 | RBCS1 P/PINS//D35S P/P450 | | 623.1 | 8 | 3 | 37.5 |
| Z-35-1 | RBCS1 P/PINS//D35S P/P450 | | 43.9 | 8 | 0 | 0.0 |

NP—Number of plants surveyed, NSP—Number of survived plants (or plants with green leaves), % SP—percentage of survived plants.
¹α-pinene level (as ratio to α-pinene production in wildtype potato) is shown as indication of terpenoid production by transgenic lines.

### Example 6

### Microbial Production or Bioconversion Using PINH

In order to demonstrate the feasability of using PINH in a microbial production or bioconversion system, PINH was expressed in Saccharomyces cerevisiae and intact transgenic yeast cells used in a bioconversion experiment. The cloning of PINH into the pYeDP60 expression vector (Pompon et al., 1996, supra) for expression in Saccharomyces cerevisiae was carried out as described under Example 3. The yeast host cells employed were WAT11U and WAT21U (Pompon et al., 1996). Both strains contain an insertion in the endogenous NADPH-cytochrome P450 reductase locus (CPR1) which is replaced by either ATR1 or ATR2, the two NADPHcytochrome P450 reductase genes from Arabidopsis thaliana (Urban et al., 1997, supra).

Transformants were selected on SGI medium, while expression was carried out in YPL medium (induction with 2% galactose) (Pompon et al., 1996). Intact cells were harvested and used for an in vivo assay, by incubation in 50 mM Tris-HCl buffer (pH 7.4) containing 0.1 mM DTT and 1 mM EDTA in the presence of 500 μM (−)-α-pinene. The reaction was allowed to proceed for 2 h at 30° C. with gentle shaking. Controls containing no substrate, and cells containing vector without insert, were also used for in vivo assays. The assays were extracted and analysed using GC-MS as described under Example 3. GC-MS analysis showed that intact yeast cells efficiently converted α-pinene into myrtenol.

The yeast cells used here to express PINH can also be modified to produce the substrate α-pinene, by introducing additional genes. For example, high-isoprenoid producing micro-organisms such as certain yeasts, could be suitable hosts, requiring only the introduction of a monoterpene synthase and perhaps a GPP synthase. Alternatively one can select an already modified host cell, such as E. coli cells described by Martin et al. 2003 (Nature Biotechnology 21: 796-802). For E. coli expression of PINH, also a heterologous NADPH-cytochrome P450 reductase is required and the N-terminal sequence of the PINH enzyme may need to be modified for correct PINH anchorage and folding, as described by Halkier et al. 1995 (supra) and elsewhere herein.

1. A recombinant host cell or organism comprising a nucleic acid sequence encoding a cytochrome P450 enzyme operably linked to a promoter active in said host cell or organism integrated into its genome, characterized in that said cell is capable of hydroxylating monoterpene substrates at the C10 carbon analogous to α-pinene, or at the C7 or C10 carbon analogous to limonene.

2. The recombinant host cell or organism according to claim 1, wherein said cytochrome P450 enzyme has at least 50% sequence identity with SEQ ID NO: 4.

3. The recombinant host cell or organism according to claim 1, wherein the substrate is selected from the group consisting of: α-pinene, limonene, α-phellandrene, α-terpinolene, α-terpinene, α-phellandrene, α-terpinene, α-terpinolene, p-menth-8-ene, p-menth-4(8)-ene, p-menth-1-ene, γ-terpinene, 3-carene, p-cymene and toluene.

4. The recombinant host cell or organism according to claim 1, wherein the host cell or organism is selected from the group consisting of: a bacterium, a virus, a fungus, an insect, a plant and a mammal cell culture.

5. The recombinant host cell or organism according to claim 4, wherein the host cell or organism is a plant and all or part of the plant comprises enhanced levels of myrtenol, myrtenal, myrtenyl acetate or other myrtenyl esters.

6. The recombinant host cell or organism according to claim 4, wherein the host cell or organism is a plant and all or part of the plant comprises enhanced levels of perilla alcohol, perilla aldehyde or perillic acid, or esters thereof.

7. The recombinant host cell or organism according to claim 4, wherein the host cell or organism is a plant and all or part of the plant comprises enhanced limonen-10-ol, limonen-10-al or dihydrolimonen-10-al.

8. The recombinant host cell or organism according to claim 5, wherein said plant has significantly enhanced resistance against plant pathogens and/or insects compared to non-recombinant control plants.

9. The recombinant host cell or organism according to claim 1, wherein said cell or organism produces myrtenol, myrtenal, myrtenyl acetate or other myrtenyl esters.

10. The recombinant host cell or organism according to claim 1, wherein said cell or organism produces perilla alcohol, perilla aldehyde or perillic acid, and esters thereof.

11. The recombinant host cell or organism according to claim 1, wherein said cell or organism produces limonen-10-ol, limonen-10-al or dihydrolimonen-10-al.

12. The recombinant host cell or organism according to claim 1, wherein said cell or organism produces benzylalcohol, benzaldehyde or esters thereof from toluene.

13. A method for hydroxylating monoterpene and aromatic substrates at the C10 carbon analogous to α-pinene, or at the C7 or C10 carbon analogous to limonene, said method comprising the steps of:
a) transforming a host cell with a nucleic acid sequence encoding a cytochrome P450 enzyme operably linked to a promoter active in said host cell,
b) culturing said recombinant cell and
c) isolating the hydroxylated monoterpene products from said culture or culture medium.

14. (canceled)

15. The recombinant host cell or organism according to claim 6, wherein said plant has significantly enhanced resistance against plant pathogens and/or insects compared to non-recombinant control plants.

16. The recombinant host cell or organism according to claim 7, wherein said plant has significantly enhanced resistance against plant pathogens and/or insects compared to non-recombinant control plants.

17. A method for the production of myrtenol, myrtenal, perilla alcohol, perilla aldehyde, perilla acid, limonene-10-ol, limonene-10-al, diliydrolimonen-10-al, benzylalcohol, benzylaladehyde or esters of these compounds comprising hydroxylating monoterpene substrates at the C10 carbon analogous to α-pinene, or at the C7 or C10 carbon analogous to limonene with a cytochrome P450 enzyme.


SEQUENCE LISTING (9 sequences)

SEQ ID NO: 1 - DNA, 1744 bp, Fragaria x ananassa
caaacaatggcggagctcatcaacaacgaaaccctttcacttgtactcctcgccgttttcctcatcctcttctacatctggtcctcatccacttccactaccagaaactcaccaccttctccaccaaaactccccattatcggaaacctccaccaactactaggctctcccggaactccacctcatcgcgcacttcaagccttatctaaactccacggccctctcatgctcctccactttggaagcttccccgtccttgtcgtctcctccgccgaggcagcacgtgagatcatgaaaacccacgaccttgctttcgccagcagacctaggaccaccgccttcgagaagcttctttacaactacaaggacgtggccgcggcgccttacggtgactactggcggcaggtgaagagcatctgcgtgctgaatctcttgagcgccaagaaggttcggtcctttcgaacccttagagaagaggagacaagatccatgatcaacaacataaaggaaacctcacgacggggagaagttgtggatgtgaggaagatggttatggggcttacgaacgacgtcatctcgagggcggctctagggaagaagtactacaatgatggagaatttaaggagctcatcaccgaatttacagagttggcgggaagtattcatattggagactatattccatcgcttggttggttgagccgtcttggcggtttggacgctaagctagtcagtttggctaaacgctacgatgcatttttggacacagtactgcaagagcatattgatagaagttcagagacgactagcaacagaaatgataaaggcgttgatgatcagaacgaggataacaaggattttgtggacgttttacttgatattcagcgggaaaactcgctccatttccctcttgacagaattagcatcaaagctgtcgtccaggacatgttccttgctgggacggatacgacgtctacacttctagagtgggcaatggcagagattctgaggcacccaagggtcatgagcaaattgcagaaagagttgaggagtgtaaaaaagggagaagaagaaatattaacagaggacgacatggttgatatgcactacttgaaggcagtgattaaggaggctcttcgtttacatcctccatttaccctacttttgcctaagatgtcgatccaagatgtgaagataaaaggttacgacatcaaggccaacacacaagtcctagtgaatgcttggcagatcggaagagacccggaatcgttcagttacaaaccggaagagtttgagccggagaggttcttggaggttaacagtggtttaagttacaaaggaactgacttcgagtttattccgttcggagctggaaggagaatttgtcctgggattcagtttgctacgactgtcaatgagattggtctagcaaatttgctgcacaagtttgattggaaattgcctggtggagtaagaaatgaggatttagacatgaatgaatcatctggtttaaccattcataagaaacaccctctcaaagccgtggctattccatattcatcagcttagtaccactggttagtggttacagtaagaagagtagatagcttgtgttctctatgttctgcagtacttaatttaaataaattgtattagctaggctgttttgtttctgaattggattcctgtagtttataataactttatatatgctgatgtttgaaataaaaaaaaaaaaaaaaaa

SEQ ID NO: 2 - DNA, 1563 bp, Fragaria x ananassa
atggcggagctcatcaacaacgaaaccctttcacttgtactcctcgccgttttcctcatcctcttctacatctggtcctcatccacttccactaccagaaactcaccaccttctccaccaaaactccccattatcggaaacctccaccaactactaggctctcccggaactccacctcatcgcgcacttcaagccttatctaaactccacggccctctcatgctcctccactttggaagcttccccgtccttgtcgtctcctccgccgaggcagcacgtgagatcatgaaaacccacgaccttgctttcgccagcagacctaggaccaccgccttcgagaagcttctttacaactacaaggacgtggccgcggcgccttacggtgactactggcggcaggtgaagagcatctgcgtgctgaatctcttgagcgccaagaaggttcggtcctttcgaacccttagagaagaggagacaagatccatgatcaacaacataaaggaaacctcacgacggggagaagttgtggatgtgaggaagatggttatggggcttacgaacgacgtcatctcgagggcggctctagggaagaagtactacaatgatggagaatttaaggagctcatcaccgaatttacagagttggcgggaagtattcatattggagactatattccatcgcttggttggttgagccgtcttggcggtttggacgctaagctagtcagtttggctaaacgctacgatgcatttttggacacagtactgcaagagcatattgatagaagttcagagacgactagcaacagaaatgataaaggcgttgatgatcagaacgaggataacaaggattttgtggacgttttacttgatattcagcgggaaaactcgctccatttccctcttgacagaattagcatcaaagctgtcgtccaggacatgttccttgctgggacggatacgacgtctacacttctagagtgggcaatggcagagattctgaggcacccaagggtcatgagcaaattgcagaaagagttgaggagtgtaaaaaagggagaagaagaaatattaacagaggacgacatggttgatatgcactacttgaaggcagtgattaaggaggctcttcgtttacatcctccatttaccctacttttgcctaagatgtcgatccaagatgtgaagataaaaggttacgacatcaaggccaacacacaagtcctagtgaatgcttggcagatcggaagagacccggaatcgttcagttacaaaccggaagagtttgagccggagaggttcttggaggttaacagtggtttaagttacaaaggaactgacttcgagtttattccgttcggagctggaaggagaatttgtcctgggattcagtttgctacgactgtcaatgagattggtctagcaaatttgctgcacaagtttgattggaaattgcctggtggagtaagaaatgaggatttagacatgaatgaatcatctggtttaaccattcataagaaacaccctctcaaagccgtggctattccatattcatcagcttag

SEQ ID NO: 3 - DNA, 1563 bp, Fragaria vesca
atggcggagctcatcaacaccgaaaccctttcacttgtactcctcgccgttttcctcatcctcttctacatctggtcctcatccacttccactaccagaaactcaccaccttctccaccaaaactccccattatcggaaacctccaccaactactaggctctcccggaactccacctcatcgcgcacttcaagccttatctaaactccacggccctctcatgctcctccactttggaagcttccccgtcctcgtcgtctcctccgccgaggcagcacgtgagatcatgaaaacccacgaccttgctttcgccagcagacctaggaccaccgccttcgagaagcttctttacaactacaaggacgtggccgcggcgccttacggtgactactggcggcaggtgaagagcatctgcgtgctgaatctcttgagcgccaagaaggttcggtcctttcgaacccttagagaagaggagacaagatccatgatcaacaacataaaggaaacctcacgacggggagaagttgtggatgtgaggaagatggttatggggcttacgaacgacgtcgtctcgagggcggctctagggaagaagtactacaatgatggagaatttaaggagctcatcaccgaatttacagagttggcgggaagtattcatattggagactatattccatcgcttggttggttgagccgtcttggcggtttggacgctaagctagtcagtttggctaaacgctacgatgcatttttggacacagtactgcaagagcatattgatagaagttcagagacgactagcaacagaaatgataaaagcgttgatgatcagaacgaggataacaaggattttgtggacgttttacttgatattcagcgggaaaactcgctccatttccctcttaacagaattagcatcaaagctgtcgtccaggacgtgttccttgctgggacggatacgacgtctacacttctagagtgggcaatggcagagattctgaggcacccaagggtcatgagcaaattgcagaaagagttgaggagtgtaaaaaagggagaagaagaaatattaacagaggacgacatggttgatatgcactacttgaaggcagtgattaaggaggctcttcgtttacatcctccatttaccctacttttgcctaagatgtcgatccaagatgtgaagataaaaggttacgacatcaaggccaacacacaagtcctagtgaatgcttggcagatcggaagagacccggaatcgttcagttacaaaccggaagagtttgaaccggaggggttcttggaggttaacagtggtttaagttacaaaggaactgacttcgagtttattccgttcggagctggaaggagaatttgtcctgggattcagtttgctacgactgtcaatgagattggtctagcaaatttgctgcacaagtttgattggaaattgcctggtggagtaagaaatgaggatttagacatgaatgaatcatctggtttaaccattcataagaaacaccctctcaaagccgtggctattccatattcatcagcttag

SEQ ID NO: 4 - protein, 520 aa, Fragaria x ananassa
MAELINNETLSLVLLAVFLILFYIWSSSTSTTRNSPPSPPKLPIIGNLHQLLGSPGTPPHRALQALSKLHGPLMLLHFGSFPVLVVSSAEAAREIMKTHDLAFASRPRTTAFEKLLYNYKDVAAAPYGDYWRQVKSICVLNLLSAKKVRSFRTLREEETRSMINNIKETSRRGEVVDVRKMVMGLTNDVISRAALGKKYYNDGEFKELITEFTELAGSIHIGDYIPSLGWLSRLGGLDAKLVSLAKRYDAFLDTVLQEHIDRSSETTSNRNDKGVDDQNEDNKDFVDVLLDIQRENSLHFPLDRISIKAVVQDMFLAGTDTTSTLLEWAMAEILRHPRVMSKLQKELRSVKKGEEEILTEDDMVDMHYLKAVIKEALRLHPPFTLLLPKMSIQDVKIKGYDIKANTQVLVNAWQIGRDPESFSYKPEEFEPERFLEVNSGLSYKGTDFEFIPFGAGRRICPGIQFATTVNEIGLANLLHKFDWKLPGGVRNEDLDMNESSGLTIHKKHPLKAVAIPYSSA

SEQ ID NO: 5 - protein, 520 aa, Fragaria vesca
MAELINTETLSLVLLAVFLILFYIWSSSTSTTRNSPPSPPKLPIIGNLHQLLGSPGTPPHRALQALSKLHGPLMLLHFGSFPVLVVSSAEAAREIMKTHDLAFASRPRTTAFEKLLYNYKDVAAAPYGDYWRQVKSICVLNLLSAKKVRSFRTLREEETRSMINNIKETSRRGEVVDVRKMVMGLTNDVVSRAALGKKYYNDGEFKELITEFTELAGSIHIGDYIPSLGWLSRLGGLDAKLVSLAKRYDAFLDTVLQEHIDRSSETTSNRNDKSVDDQNEDNKDFVDVLLDIQRENSLHFPLNRISIKAVVQDVFLAGTDTTSTLLEWAMAEILRHPRVMSKLQKELRSVKKGEEEILTEDDMVDMHYLKAVIKEALRLHPPFTLLLPKMSIQDVKIKGYDIKANTQVLVNAWQIGRDPESFSYKPEEFEPEGFLEVNSGLSYKGTDFEFIPFGAGRRICPGIQFATTVNEIGLANLLHKFDWKLPGGVRNEDLDMNESSGLTIHKKHPLKAVAIPYSSA

SEQ ID NO: 6 - protein, 6 aa, Fragaria vesca
AGTDTT

SEQ ID NO: 7 - protein, 11 aa, Fragaria vesca
PFGAGRRICPG

SEQ ID NO: 8 - DNA, 30 bp, Artificial Sequence
  Description of Artificial Sequence Synthetic oligonucleotide
cagatctatggaagccacttcttgggttac

SEQ ID NO: 9 - DNA, 27 bp, Artificial Sequence
  Description of Artificial Sequence Synthetic oligonucleotide
ccttaagagaagctagtagctggaacc